SEQUENCE LISTING (133 sequences)

SEQ ID NO: 1 - DNA, 23 bp, synthetic construct
  misc_feature 1..23: note = Guide RNA recognition element
  source 1..23: mol_type = other DNA
agctactctggcccttatagtcc

SEQ ID NO: 2 - RNA, 23 nt, synthetic construct
  misc_feature 1..23: note = Guide RNA
  source 1..23: mol_type = other RNA
tcgatgtgaccgggaatatcagg

SEQ ID NO: 3 - DNA, 23 bp, synthetic construct
  misc_feature 1..23: note = Guide RNA recognition element
  source 1..23: mol_type = other DNA
actaaatctgccatacgttgtcc

SEQ ID NO: 4 - RNA, 23 nt, synthetic construct
  misc_feature 1..23: note = Guide RNA
  source 1..23: mol_type = other RNA
tgatttagacggtatgcaacagg

SEQ ID NO: 5 - DNA, 28 bp, synthetic construct
  misc_feature 1..28: note = Primer
  source 1..28: mol_type = other DNA
gtagtggtgccttagagcttactcatcc

SEQ ID NO: 6 - DNA, 27 bp, synthetic construct
  misc_feature 1..27: note = Primer
  source 1..27: mol_type = other DNA
ctagcatgggcagtactcatgactaag

SEQ ID NO: 7 - DNA, 2446 bp, Homo sapiens
  source 1..2446: mol_type = other DNA
gattaccgagtgccggggagcccggaggagccgccgacgcagccgccaccgccgccgccgccgccaccagagccgccctgtccgcgccgcgcctcggcagccggaacagggccgccgtcggggagccccaacacacggtccacagctcatcatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaa

SEQ ID NO: 8 - protein, 605 aa, Homo sapiens
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 9 - DNA, 23 bp, synthetic construct
  misc_feature 1..23: note = Guide RNA
  source 1..23: mol_type = other DNA
tcgatgtgaccgggaatatcagg

SEQ ID NO: 10 - DNA, 23 bp, synthetic construct
  misc_feature 1..23: note = Guide RNA
  source 1..23: mol_type = other DNA
tgatttagacggtatgcaacagg

SEQ ID NO: 11 - DNA, 23 bp, synthetic construct
  misc_feature 1..23: note = Guide RNA
  source 1..23: mol_type = other DNA
aagtacaaagcatctgatttggg

SEQ ID NO: 12 - DNA, 23 bp, synthetic construct
  misc_feature 1..23: note = Guide RNA
  source 1..23: mol_type = other DNA
agcatctgatttgggaatgtggg

SEQ ID NO: 13 - DNA, 23 bp, synthetic construct
  misc_feature 1..23: note = Guide RNA
  source 1..23: mol_type = other DNA
tggatttgattgacatactttgg

SEQ ID NO: 14 - DNA, 23 bp, synthetic construct
  misc_feature 1..23: note = Guide RNA
  source 1..23: mol_type = other DNA
gcttcttacttttgggaacaagg

SEQ ID NO: 15 - DNA, 25 bp, synthetic construct
  misc_feature 1..25: note = Guide RNA
  source 1..25: mol_type = other DNA
tttgattgacatactttggaggcaa

SEQ ID NO: 16 - DNA, 25 bp, synthetic construct
  misc_feature 1..25: note = Guide RNA
  source 1..25: mol_type = other DNA
ttttccttgttcccaaaagtaagaa

SEQ ID NO: 17 - DNA, 23 bp, synthetic construct
  misc_feature 1..23: note = Guide RNA recognition sequence
  source 1..23: mol_type = other DNA
aaactaactgtatgaaacctccc

SEQ ID NO: 18 - RNA, 23 nt, synthetic construct
  misc_feature 1..23: note = Guide RNA
  source 1..23: mol_type = other RNA
tttgattgacatactttggaggg

SEQ ID NO: 19 - DNA, 23 bp, synthetic construct
  misc_feature 1..23: note = Guide RNA recognition element
  source 1..23: mol_type = other DNA
gaatgaggttctagatatagacc

SEQ ID NO: 20 - RNA, 23 nt, synthetic construct
  misc_feature 1..23: note = Guide RNA
  source 1..23: mol_type = other RNA
cttactccaagatctatatctgg

SEQ ID NO: 21 - DNA, 23 bp, synthetic construct
  misc_feature 1..23: note = Guide RNA recognition sequence
  source 1..23: mol_type = other DNA
tatgaaacctccgttctatatcc

SEQ ID NO: 22 - RNA, 23 nt, synthetic construct
  misc_feature 1..23: note = Guide RNA
  source 1..23: mol_type = other RNA
atactttggaggcaagatatagg

SEQ ID NO: 23 - DNA, 23 bp, synthetic construct
  misc_feature 1..23: note = Guide RNA recognition element
  source 1..23: mol_type = other DNA
tccgttctatatctagaacctcc

SEQ ID NO: 24 - RNA, 23 nt, synthetic construct
  misc_feature 1..23: note = Guide RNA
  source 1..23: mol_type = other RNA
aggcaagatatagatcttggagg

SEQ ID NO: 25 - DNA, 23 bp, synthetic construct
  misc_feature 1..23: note = Guide RNA recognition element
  source 1..23: mol_type = other DNA
ctatatctagaacctcattcacc

SEQ ID NO: 26 - RNA, 23 nt, synthetic construct
  misc_feature 1..23: note = Guide RNA
  source 1..23: mol_type = other RNA
gatatagatcttggagtaagtgg

SEQ ID NO: 27 - DNA, 23 bp, synthetic construct
  misc_feature 1..23: note = Guide RNA recognition element
  source 1..23: mol_type = other DNA
actgacttcagtttatgaagacc

SEQ ID NO: 28 - RNA, 23 nt, synthetic construct
  misc_feature 1..23: note = Guide RNA
  source 1..23: mol_type = other RNA
tgactgaagtcaaatacttctgg

SEQ ID NO: 29 - DNA, 23 bp, synthetic construct
  misc_feature 1..23: note = Guide RNA recognition element
  source 1..23: mol_type = other DNA
aagtagatcaacattgactcgcc

SEQ ID NO: 30 - RNA, 23 nt, synthetic construct
  misc_feature 1..23: note = Guide RNA
  source 1..23: mol_type = other RNA
ttcatctagttgtaactgagcgg

SEQ ID NO: 31 - DNA, 23 bp, synthetic construct
  misc_feature 1..23: note = Guide RNA recognition element
  source 1..23: mol_type = other DNA
taagtggacagagaagtagatcc

SEQ ID NO: 32 - RNA, 23 nt, synthetic construct
  misc_feature 1..23: note = Guide RNA
  source 1..23: mol_type = other RNA
attcacctgtctcttcatctagg

SEQ ID NO: 33 - DNA, 23 bp, synthetic construct
  misc_feature 1..23: note = Guide RNA recognition element
  source 1..23: mol_type = other DNA
gagtcaatgttgatctacttccc

SEQ ID NO: 34 - RNA, 23 nt, synthetic construct
  misc_feature 1..23: note = Guide RNA
  source 1..23: mol_type = other RNA
ctcagttacaactagatgaaggg

SEQ ID NO: 35 - DNA, 23 bp, synthetic construct
  misc_feature 1..23: note = Guide RNA recognition element
  source 1..23: mol_type = other DNA
acttaaccctctttaagtggacc

SEQ ID NO: 36 - RNA, 23 nt, synthetic construct
  misc_feature 1..23: note = Guide RNA
  source 1..23: mol_type = other RNA
tgaattgggagaaattcacctgg

SEQ ID NO: 37 - DNA, 23 bp, synthetic construct
  misc_feature 1..23: note = Guide RNA recognition element
  source 1..23: mol_type = other DNA
gttgatctacttctctgtccacc

SEQ ID NO: 38 - RNA, 23 nt, synthetic construct
  misc_feature 1..23: note = Guide RNA
  source 1..23: mol_type = other RNA
caactagatgaagagacaggtgg

SEQ ID NO: 39 - DNA, 23 bp, synthetic construct
  misc_feature 1..23: note = Guide RNA recognition element
  source 1..23: mol_type = other DNA
tattatcgaggagggtttgaacc

SEQ ID NO: 40 - RNA, 23 nt, synthetic construct
  misc_feature 1..23: note = Guide RNA
  source 1..23: mol_type = other RNA
ataatagctcctcccaaacttgg

SEQ ID NO: 41 - DNA, 23 bp, Homo sapiens
  source 1..23: mol_type = other DNA
tttgattgacatactttggaggg

SEQ ID NO: 42 - DNA, 23 bp, Homo sapiens
  source 1..23: mol_type = other DNA
cttactccaagatctatatctgg

SEQ ID NO: 43 - DNA, 23 bp, Homo sapiens
  source 1..23: mol_type = other DNA
atactttggaggcaagatatagg

SEQ ID NO: 44 - DNA, 23 bp, Homo sapiens
  source 1..23: mol_type = other DNA
aggcaagatatagatcttggagg

SEQ ID NO: 45 - DNA, 23 bp, Homo sapiens
  source 1..23: mol_type = other DNA
gatatagatcttggagtaagtgg

SEQ ID NO: 46 - DNA, 23 bp, Homo sapiens
  source 1..23: mol_type = other DNA
tgactgaagtcaaatacttctgg

SEQ ID NO: 47 - DNA, 23 bp, Homo sapiens
  source 1..23: mol_type = other DNA
ttcatctagttgtaactgagcgg

SEQ ID NO: 48 - DNA, 23 bp, Homo sapiens
  source 1..23: mol_type = other DNA
attcacctgtctcttcatctagg

SEQ ID NO: 49 - DNA, 23 bp, Homo sapiens
  source 1..23: mol_type = other DNA
ctcagttacaactagatgaaggg

SEQ ID NO: 50 - DNA, 23 bp, Homo sapiens
  source 1..23: mol_type = other DNA
tgaattgggagaaattcacctgg

SEQ ID NO: 51 - DNA, 23 bp, Homo sapiens
  source 1..23: mol_type = other DNA
caactagatgaagagacaggtgg

SEQ ID NO: 52 - DNA, 23 bp, Homo sapiens
  source 1..23: mol_type = other DNA
ataatagctcctcccaaacttgg

SEQ ID NO: 53 - DNA, 22 bp, synthetic construct
  misc_feature 1..22: note = Guide RNA recognition sequence
  source 1..22: mol_type = other DNA
aaaaagcgagtaatgttgatcc

SEQ ID NO: 54 - RNA, 23 nt, synthetic construct
  misc_feature 1..23: note = Guide RNA
  source 1..23: mol_type = other RNA
tttttcgctcagttacaactagg

SEQ ID NO: 55 - DNA, 23 bp, synthetic construct
  misc_feature 1..23: note = Guide RNA recognition sequence
  source 1..23: mol_type = other DNA
gttgatctacttctctgtccacc

SEQ ID NO: 56 - RNA, 23 nt, synthetic construct
  misc_feature 1..23: note = Guide RNA
  source 1..23: mol_type = other RNA
caactagatgaagagacaggtgg

SEQ ID NO: 57 - DNA, 23 bp, Homo sapiens
  source 1..23: mol_type = other DNA
tttttcgctcagttacaactagg

SEQ ID NO: 58 - DNA, 23 bp, Homo sapiens
  source 1..23: mol_type = other DNA
caactagatgaagagacaggtgg

SEQ ID NO: 59 - DNA, 23 bp, synthetic construct
  misc_feature 1..23: note = Guide RNA
  source 1..23: mol_type = other DNA
caagatatagatcttggagtaag

SEQ ID NO: 60 - DNA, 22 bp, synthetic construct
  misc_feature 1..22: note = Guide RNA
  source 1..22: mol_type = other DNA
aagatatagatcttggagtaag

SEQ ID NO: 61 - DNA, 20 bp, synthetic construct
  misc_feature 1..20: note = Guide RNA
  source 1..20: mol_type = other DNA
gatatagatcttggagtaag

SEQ ID NO: 62 - DNA, 18 bp, synthetic construct
  misc_feature 1..18: note = Guide RNA
  source 1..18: mol_type = other DNA
tatagatcttggagtaag

SEQ ID NO: 63 - DNA, 17 bp, synthetic construct
  misc_feature 1..17: note = Guide RNA
  source 1..17: mol_type = other DNA
atagatcttggagtaag

SEQ ID NO: 64 - DNA, 23 bp, synthetic construct
  misc_feature 1..23: note = Guide RNA
  source 1..23: mol_type = other DNA
tggaggcaagatatagatcttgg

SEQ ID NO: 65 - DNA, 200 bp, synthetic construct
  misc_feature 1..200: note = ssDNA template
  source 1..200: mol_type = other DNA
ttaaaaaacatgagctctctccttcctttttttgtcttaaacataggacatggatttgattgacatactttggaggcaagatatagatcttggagtaagtggagaagtatttgacttcagtcagcgacggaaagagtatgagctggaaaaacagaaaaaacttgaaaaggaaagacaagaacaactccaaaaggagcaag

SEQ ID NO: 66 - DNA, 23 bp, synthetic construct
  misc_feature 1..23: note = Guide RNA
  source 1..23: mol_type = other DNA
tcgatgtgaccgggaatatcagg

SEQ ID NO: 67 - DNA, 23 bp, synthetic construct
  misc_feature 1..23: note = Guide RNA
  source 1..23: mol_type = other DNA
tgatttagacggtatgcaacagg

SEQ ID NO: 68 - DNA, 23 bp, synthetic construct
  misc_feature 1..23: note = gRNA1 recognition element
  source 1..23: mol_type = other DNA
cctgatattcccggtcacatcga

SEQ ID NO: 69 - DNA, 23 bp, synthetic construct
  misc_feature 1..23: note = gRNA1 recognition element
  source 1..23: mol_type = other DNA
ggactataagggccagtgtagct

SEQ ID NO: 70 - DNA, 25 bp, synthetic construct
  misc_feature 1..25: note = gRNA
  source 1..25: mol_type = other DNA
caccgtcgatgtgaccgcgaatatc

SEQ ID NO: 71 - DNA, 25 bp, synthetic construct
  misc_feature 1..25: note = gRNA
  source 1..25: mol_type = other DNA
cagctacactggcccttatagcaaa

SEQ ID NO: 72 - DNA, 23 bp, synthetic construct
  misc_feature 1..23: note = gRNA2 recognition element
  source 1..23: mol_type = other DNA
tgatttagacggtatgcaacagg

SEQ ID NO: 73 - DNA, 23 bp, synthetic construct
  misc_feature 1..23: note = gRNA2 recognition element
  source 1..23: mol_type = other DNA
actaaatctgccatacgttgtcc

SEQ ID NO: 74 - DNA, 25 bp, synthetic construct
  misc_feature 1..25: note = gRNA
  source 1..25: mol_type = other DNA
caccgtgatttagacggtatgcaac

SEQ ID NO: 75 - DNA, 25 bp, synthetic construct
  misc_feature 1..25: note = gRNA
  source 1..25: mol_type = other DNA
cactaaatctgccatacgttgcaaa

SEQ ID NO: 76 - DNA, 23 bp, synthetic construct
  misc_feature 1..23: note = gRNA
  source 1..23: mol_type = other DNA
tcgatgtgaccgggaatatcagg

SEQ ID NO: 77 - DNA, 23 bp, synthetic construct
  misc_feature 1..23: note = gRNA
  source 1..23: mol_type = other DNA
tgatttagacggtatgcaacagg

SEQ ID NO: 78 - DNA, 41 bp, synthetic construct
  misc_feature 1..41: note = gRNA
  misc_feature 23..24: note = n is a, c, g, or t
  misc_feature 27: note = n is a, c, g, or t
  misc_feature 32..35: note = n is a, c, g, or t
  misc_feature 41: note = n is a, c, g, or t
  source 1..41: mol_type = other DNA
tcagtcacttgttcctgataaannggnaattnnnnaagttn

SEQ ID NO: 79 - DNA, 45 bp, synthetic construct
  misc_feature 1..45: note = gRNA
  source 1..45: mol_type = other DNA
acgtttcagtcacttgttcccgatcacatcgagagcccagtcttc

SEQ ID NO: 80 - DNA, 172 bp, Homo sapiens
  source 1..172: mol_type = other DNA
ctctctccttcctttttttgtctttttcataggacatggatttgattgacatactttggaggcaagtttttgatcttggagtaagtcgagaagtttttgacttcagtcagcgacggaaagagtatgagctggtttttcagaaaaaacttgaaaaggaaagacaagaacaact

SEQ ID NO: 81 - DNA, 171 bp, Homo sapiens
  source 1..171: mol_type = other DNA
agagaggaagcaaaaaaacagaaaaactatcctgtacctaaactaactgtatgaaacctcccttcaaaaactagaacctcattcagctcttcaaaaactgaagtcagtcgctccctatctcatactcgaccaaaaagtctaaaaagaactattccttactgttcttcttga

SEQ ID NO: 82 - DNA, 26 bp, synthetic construct
  misc_feature 1..26: note = gRNA
  source 1..26: mol_type = other DNA
caagatatagatcttggagtaagtgg

SEQ ID NO: 83 - DNA, 25 bp, synthetic construct
  misc_feature 1..25: note = gRNA
  source 1..25: mol_type = other DNA
aagatatagatcttggagtaagtgg

SEQ ID NO: 84 - DNA, 23 bp, synthetic construct
  misc_feature 1..23: note = gRNA
  source 1..23: mol_type = other DNA
gatatagatcttggagtaagtgg

SEQ ID NO: 85 - DNA, 21 bp, synthetic construct
  misc_feature 1..21: note = gRNA
  source 1..21: mol_type = other DNA
tatagatcttggagtaagtgg

SEQ ID NO: 86 - DNA, 20 bp, synthetic construct
  misc_feature 1..20: note = gRNA
  source 1..20: mol_type = other DNA
atagatcttggagtaagtgg

SEQ ID NO: 87 - DNA, 26 bp, synthetic construct
  misc_feature 1..26: note = gRNA
  source 1..26: mol_type = other DNA
caagatatagatcttggagtaagtgg

SEQ ID NO: 88 - DNA, 25 bp, synthetic construct
  misc_feature 1..25: note = gRNA
  source 1..25: mol_type = other DNA
aagatatagatcttggagtaagtgg

SEQ ID NO: 89 - DNA, 23 bp, synthetic construct
  misc_feature 1..23: note = gRNA
  source 1..23: mol_type = other DNA
gatatagatcttggagtaagtgg

SEQ ID NO: 90 - DNA, 21 bp, synthetic construct
  misc_feature 1..21: note = gRNA
  source 1..21: mol_type = other DNA
tatagatcttggagtaagtgg

SEQ ID NO: 91 - DNA, 20 bp, synthetic construct
  misc_feature 1..20: note = gRNA
  source 1..20: mol_type = other DNA
atagatcttggagtaagtgg

SEQ ID NO: 92 - DNA, 120 bp, Homo sapiens
  source 1..120: mol_type = other DNA
tttttttgtcttaaacataggacatggatttgattgacatactttggaggcaagatatagatcttggagtaagtcgagaagtatttgacttcagtcagcgacggaaagagtatgagctgg

SEQ ID NO: 93 - DNA, 120 bp, Homo sapiens
  source 1..120: mol_type = other DNA
aaaaaaacagaatttgtatcctgtacctaaactaactgtatgaaacctccgttctatatctagaacctcattcagctcttcataaactgaagtcagtcgctgcctttctcatactcgacc

SEQ ID NO: 94 - DNA, 120 bp, Homo sapiens
  source 1..120: mol_type = other DNA
aaaaacagaaaaaacttgaaaaggaaagacaagaacaactccaaaaggagcaagagaaagcctttttcgctcagttacaactagatgaagagacaggtgaatttctcccaattcagccag

SEQ ID NO: 95 - DNA, 120 bp, Homo sapiens
  source 1..120: mol_type = other DNA
tttttgtcttttttgaacttttcctttctgttcttgttgaggttttcctcgttctctttcggaaaaagcgagtcaatgttgatctacttctctgtccacttaaagagggttaagtcggtc

SEQ ID NO: 96 - DNA, 138 bp, Homo sapiens
  source 1..138: mol_type = other DNA
taggacatggatttgattgacatactttggaggcaagatatagatcttggagtaagaagtatttgacttcagtcagcgacggaaagagtatgagctggaaaaacagaaaaaacttgaaaaggaaagacaagaacaact

SEQ ID NO: 97 - DNA, 143 bp, Homo sapiens
  source 1..143: mol_type = other DNA
atcctgtacctaaactaactgtatgaaacctccgttctatatctagaacctcattcagctcttcataaactgaagtcagtcgctgcctttctcatactcgacctttttgtcttttttgaacttttcctttctgttcttgttga

SEQ ID NO: 98 - DNA, 138 bp, Homo sapiens; identical to SEQ ID NO: 96
  source 1..138: mol_type = other DNA

SEQ ID NO: 99 - DNA, 143 bp, Homo sapiens
  source 1..143: mol_type = other DNA
atcctgtacctaaactaactgtatgaaacctccgttctatatctagaacctcattcacctcttcataaactgaagtcagtcgctgcctttctcatactcgacctttttgtcttttttgaacttttcctttctgttcttgttga

SEQ ID NO: 100 - DNA, 159 bp, Homo sapiens
  source 1..159: mol_type = other DNA
gacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaa

SEQ ID NO: 101 - DNA, 156 bp, Homo sapiens
  source 1..156: mol_type = other DNA
gacatggatttgattgacatactttggaggcaagatatagatcttggagtaaggagaagtatttgacttcagtcaggacggaaagagtatgagctggaaaaacagaaaaaacttgaaaaggaaagacaagaacaactccaaaaggagcaagagaaa

SEQ ID NO: 102 - DNA, 159 bp, Homo sapiens; identical to SEQ ID NO: 100
  source 1..159: mol_type = other DNA

SEQ ID NO: 103 - DNA, 81 bp, Homo sapiens
  source 1..81: mol_type = other DNA
ctttggaggaagatatagatcttggagtaagtcgagaagtatttgacttcagtagcgacggaaagagtatgagctggaaaa

SEQ ID NO: 104 - DNA, 79 bp, Homo sapiens
  source 1..79: mol_type = other DNA
ctttggaggcaagatatagaggagtaagtggagaagtatttgacttcagtcagcgacggaaagagtatgagctggaaaa

SEQ ID NO: 105 - DNA, 83 bp, Homo sapiens
  source 1..83: mol_type = other DNA
ctttggaggcaagatatagatcttggagtaagtcgagaagtatttgacttcagtcagcgacggaaagagtatgagctggaaaa

SEQ ID NO: 106 - DNA, 81 bp, Homo sapiens
  source 1..81: mol_type = other DNA
ctttggaggcaagatatagattggagtaagtggagaagtatttgacttcagtcagcgacggaaagagtatgagctggaaaa

SEQ ID NO: 107 - DNA, 81 bp, Homo sapiens
  source 1..81: mol_type = other DNA
ctttggggcagatatagatcttggagtaagtcgagaagtatttgacttcagtcagcgacggaaagagtatgagctggaaaa

SEQ ID NO: 108 - DNA, 76 bp, Homo sapiens
  source 1..76: mol_type = other DNA
ctttggaggcaagatcttggagtaagtcgagaagtatttgacttcagtcagcgacggaaagagtatgagctggaaa

SEQ ID NO: 109 - DNA, 79 bp, Homo sapiens
  source 1..79: mol_type = other DNA
ctttggaggcaagatatagaggagtaagtcgagaagtatttgacttcagtcagcgacggaaagagtatgagctggaaaa

SEQ ID NO: 110 - DNA, 83 bp, Homo sapiens
  source 1..83: mol_type = other DNA
acatactttggaggcaagatatagatcttggagtaagtcgagaagtatttgacttcagtcagcgacggaaagagtatgagctg

SEQ ID NO: 111 - DNA, 83 bp, Homo sapiens
  source 1..83: mol_type = other DNA
acatactttggaggcaagatatagatcttggagtaagtggagaagtatttgacttcagtcagcgacggaaagagtatgagctg

SEQ ID NO: 112 - DNA, 63 bp, Homo sapiens
  source 1..63: mol_type = other DNA
acatactttggagtaagtcgagaagtatttgacttcagtcagcgacggaaagagtatgagctg

SEQ ID NO: 113 - DNA, 76 bp, synthetic construct
  misc_feature 1..76: note = gRNA
  source 1..76: mol_type = other DNA
gttttagagctagaaatagcaagttaaaataaggctagtccgttatcaacttgaaaaagtggcaccgagtcggtgc

SEQ ID NO: 114 - protein, 1367 aa, synthetic construct
  REGION 1..1367: note = Cas9
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 115 - DNA, 4101 bp, synthetic construct
  misc_feature 1..4101: note = Cas9
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 116 - DNA, 39 bp, synthetic construct
  misc_feature 1..39: note = minicmv promoter
  source 1..39: mol_type = other DNA
ggtaggcgtgtacggtgggaggcctatataagcagagct

SEQ ID NO: 117 - DNA, 212 bp, synthetic construct
  misc_feature 1..212: note = Ef1a promoter
  source 1..212: mol_type = other DNA
gggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 118 - DNA, 241 bp, synthetic construct
  misc_feature 1..241: note = U6 promoter
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 119 - DNA, 508 bp, synthetic construct
  misc_feature 1..508: note = CMV promoter
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 120 - DNA, 130 bp, synthetic construct
  misc_feature 1..130: note = AAV2 ITR
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 121 - DNA, 21 bp, synthetic construct
  misc_feature 1..21: note = SV40 NLS
  source 1..21: mol_type = other DNA
ccgaagaaaaagcgcaaggtc

SEQ ID NO: 122 - DNA, 221 bp, synthetic construct
  misc_feature 1..221: note = H1 promoter
  source 1..221: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatct

SEQ ID NO: 123 - DNA, 267 bp, synthetic construct
  misc_feature 1..267: note = 7SK promoter
  source 1..267: mol_type = other DNA
catatgccatgggctgccgctctagactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacc

SEQ ID NO: 124 - DNA, 96 bp, synthetic construct
  misc_feature 1..96: note = Full length guide RNA DNA sequence
  source 1..96: mol_type = other DNA
gatatagatcttggagtaaggttttagagctagaaatagcaagttaaaataaggctagtccgttatcaacttgaaaaagtggcaccgagtcggtgc

SEQ ID NO: 125 - DNA, 1041 bp, synthetic construct
  misc_feature 1..1041: note = guide RNA expression cassette
  source 1..1041: mol_type = other DNA
cgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatatagatcttggagtaaggttttagagctagaaatagcaagttaaaataaggctagtccgttatcaacttgaaaaagtggcaccgagtcggtgctttttttgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctgatatagatcttggagtaaggttttagagctagaaatagcaagttaaaataaggctagtccgttatcaacttgaaaaagtggcaccgagtcggtgcttttttcatatgccatgggctgccgctctagactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtaccgatatagatcttggagtaaggttttagagctagaaatagcaagttaaaataaggctagtccgttatcaacttgaaaaagtggcaccgagtcggtgc

SEQ ID NO: 126 - RNA, 20 nt, synthetic construct
  misc_feature 1..20: note = gRNA DNA binding domain
  source 1..20: mol_type = other RNA
cttactccaagatctatatc

SEQ ID NO: 127 - RNA, 76 nt, synthetic construct
  misc_feature 1..76: note = RNA scaffold RNA sequence
  source 1..76: mol_type = other RNA
gcaccgactcggtgccactttttcaagttgataacggactagccttattttaacttgctatttctagctctaaaac

SEQ ID NO: 128 - RNA, 96 nt, synthetic construct
  misc_feature 1..96: note = guide RNA
  source 1..96: mol_type = other RNA
cttactccaagatctatatcgcaccgactcggtgccactttttcaagttgataacggactagccttattttaacttgctatttctagctctaaaac

SEQ ID NO: 129 - DNA, 9905 bp, Homo sapiens
  source 1..9905: mol_type = other DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 130 - protein, 1210 aa, Homo sapiens [sequence omitted]

SEQ ID NO: 131 - DNA, 20 bp, synthetic construct
  misc_feature 1..20: note = scrambled gRNA
  source 1..20: mol_type = other DNA
gcactaccagagctaactca

SEQ ID NO: 132 - DNA, 26 bp, synthetic construct
  misc_feature 1..26: note = Neh2 forward primer
  source 1..26: mol_type = other DNA
caccatcaacagtggcataatgtgaa

SEQ ID NO: 133 - DNA, 27 bp, synthetic construct
  misc_feature 1..27: note = Neh2 reverse primer
  source 1..27: mol_type = other DNA
aactcaggttaggtactgaactcatca

(19) United States
(12) Patent Application Publication
(10) Pub. No.: US 2025/0018060 A1
(43) Pub. Date: Jan. 16, 2025

(54) ADENOVIRUS DELIVERY SYSTEM FOR CANCER TREATMENT

(71) Applicant: Christiana Care Gene Editing Institute, Inc., Wilmington, DE (US)

(72) Inventors: Eric B. Kmiec, Middletown, DE (US); Byung-chun Yoo, Newark, DE (US); Steven Yang, Chapel Hill, NC (US); Kelly H. Banas, Bear, DE (US)

(73) Assignee: Christiana Care Gene Editing Institute, Inc., Wilmington, DE (US)

(21) Appl. No.: 18/711,355

(22) PCT Filed: Nov. 18, 2022
(86) PCT No.: PCT/US2022/050453

§ 371 Date: May 17, 2024

Related U.S. Application Data
(60) Provisional application No. 63/281,354, filed on Nov. 19, 2021.

(51) Int. Cl. A61K 48/00; A61K 31/337; A61K 31/475; A61K 31/555; A61K 33/243; A61P 35/00; C12N 9/22; C12N 15/11; C12N 15/86

(52) U.S. Cl. CPC A61K 48/0058; A61K 31/337; A61K 31/475; A61K 31/555; A61K 33/243; A61P 35/00; C12N 9/22; C12N 15/111; C12N 15/86; C07K 2319/09; C12N 2310/20; C12N 2710/10143; C12N 2800/40

(57) ABSTRACT

The disclosure provides recombinant adenoviruses comprising a polynucleotide comprising: a first DNA sequence encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in a gene; and a first promoter that is operably linked to the DNA sequence. The disclosure also provides pharmaceutical compositions comprising the adenoviruses described herein. The disclosure further provides a method of treating cancer in a subject, the method comprising administering to the subject a therapeutically effective amount of the pharmaceutical compositions as described herein to the subject.

### RELATED APPLICATIONS

This application claims priority to U.S. Provisional Application No. 63/281,354, filed on Nov. 19, 2021, the entire contents of which are expressly incorporated herein by reference.

### SEQUENCE LISTING

The Sequence Listing associated with this application is filed in electronic format and is hereby incorporated by reference into the specification in its entirety. The name of the text file containing the Sequence Listing is 130949_01220_Sequence_Listing. The size of the text file is 179,194 bytes, and the text file was created on Nov. 18, 2022.

### BACKGROUND

Cancer is presently one of the leading causes of death in developed nations. A diagnosis of cancer traditionally involves serious health complications. Cancer can cause disfigurement, chronic or acute pain, lesions, organ failure, or even death. Commonly diagnosed cancers include lung cancer, pancreatic cancer, breast cancer, melanoma, lymphoma, carcinoma, sarcoma leukemia, endometrial cancer, colon and rectal cancer, prostate cancer, and bladder cancer. Traditionally, many cancers are treated with surgery, chemotherapy, radiation, or combinations thereof.

Nuclear factor (erythroid-derived 2)-like 2, also known as NFE2L2 or NRF2, is a transcription factor that in humans is encoded by the NFE2L2 gene. NRF2 is a basic leucine zipper (bZIP) protein that regulates the expression of antioxidant proteins that protect against oxidative damage triggered by injury and inflammation.

A recent study catalogued somatic NRF2 mutations in various cancer cases reported in The Cancer Genome Atlas. Kerins, M. J. & Ooi, A, Sci. Rep. 8, Article No. 12846 (2018). This study identified the percentage of NRF2 and KEAP1 mutations found across 33 different tumor types as well as the common mutations responsible for constitutive NRF2 activation. The study reported 214 cases of NRF2 mutations, with the most cases seen in Lung Squamous Cell Carcinoma. The NRF2 mutations commonly seen among the tumor types are found within the Neh2 Domain of the protein, which is known as the KEAP1 binding domain. KEAP1 is a negative regulator of NRF2 and mediates NRF2 degradation under basal conditions. The mutations reported in the study cause loss of KEAP1 binding and lead to constitutive expression of NRF2 in cancerous cells. The most common mutation reported in LUSC. R34G, is especially of interest because this mutation forms a new PAM site for Cas9 recognition. The first base of codon 34 in NRF2 is mutated from cytosine to guanine. This new PAM site allows for differentiation of cancerous and noncancerous cells. There have been several other mutations reported within the Neh2 Domain of NRF2, which form new PAM sites as well. Frank, R. et al., Clin. Cancer Res. 24:3087-96 (2018); Menegon, S., Columbano, A. & Giordano, S. The Dual Roles of NRF2 in Cancer. Trends in Molecular Medicine (2016); Shibata, T. et al., Proc Natl Acad Sci USA 105:13568-73 (2008). Similar experiments have been carried out using known mutations as new recognition sites for CRISPR/Cas9. Cheung. A. H. K. et al., Lab. Investig. 98:968-76 (2018).

Chemotherapeutic agents used in the treatment of cancer are known to produce several serious and unpleasant side effects in patients. For example, some chemotherapeutic agents cause neuropathy, nephrotoxicity, stomatitis, alopecia, decreased immunity, anemia, cardiotoxicity, fatigue, neuropathy, myelosuppression, or combinations thereof. Oftentimes, chemotherapy is not effective, or loses effectiveness after a period of efficacy, either during treatment, or shortly after the treatment regimen concludes.

Thus a need exists for improved methods of treating cancer.

### SUMMARY OF THE INVENTION

In certain aspects, the disclosure relates to an adenovirus comprising a polynucleotide, wherein the polynucleotide comprises: (a) a first DNA molecule encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in a gene; and (b) a first promoter that is operably linked to the DNA molecule. In certain embodiments, the gene is a wildtype gene. In certain embodiments, the gene is a variant gene. In certain embodiments, the gene is selected from the group consisting of NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7, BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNX1T1, DNMT3A, SMC1A, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST, TSC2, EEF1A1, WHSC1, APOB, NUP133, AXIN1, PHF6, TET2, WT1, FLT3, FLT4, SMC3, CEBPA, RAD21, RAD50, RAD51, PTPDC1, ASXL1, EZH2, NPM1, SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA, LATS1, POLRMT, CDK4, COL5A1, PPP6C, MECOM, DACH1, MAP2K1, MAP2K2, RQCD1, DDX3X, NUP93, PPMID, CHD2, CHD3, CCND1, CCND2, CCND3, ACVR1, KMT2A, KMT2B, KMT2C, KMT2D, SIN3A, SCAF4, DICER1, FOXA2, CTNND1, MYC, MYCL, MYCN, SOX17, ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF2I, RIT1, MGA, ABL1, BRAF, CHEK1, FANCC, JAK2, MITF, PDCDILG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSFIR, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKBIA, PRDM1, DOT1L, FGFR1, LMO1, NKX2-1, PREX2, HSD3B1, LRP1B, TSHR, ATRX, CDC73, HSP90AA1, PRKCI, AURKA, PRKDC, VEGFA, AURKB, CDK12, FH, MAGI2, PRSS8, SMO, FLCN, IGF1R, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B, PARK2, RANBP2, SRC, IRS2, PAX5, RARA, BLM, FANCA, JAK1, FCRL4, LIG4, MAR, PWWP3A, MUC16, MUC17, FCGBP, FAT17, MMSET, IRTA2, TTN, DST, and STAT3.

In certain embodiments, the gene is an NRF2 gene. In certain embodiments, the NRF2 gene encodes an NRF2 polypeptide comprising one or more amino acid substitutions selected from the group consisting of Q26E, Q26P, D29G, V32G, R34G, R34P, F71S, Q75H, E79G, T80P, E82W, and E185D relative to the amino acid sequence of SEQ ID NO: 8. In certain embodiments, the NRF2 polypeptide comprises an R34G substitution relative to the amino acid sequence of SEQ ID NO: 8. In certain embodiments, the gRNA is complementary to a target sequence in exon 2 of the NRF2 gene. In certain embodiments, the polynucleotide further comprises: (a) a second DNA molecule encoding the gRNA; and (b) a second promoter that is operably linked to the second DNA molecule.

In certain embodiments, the polynucleotide further comprises: (a) a third DNA molecule encoding the gRNA; and (b) a third promoter that is operably linked to the third DNA molecule. In certain embodiments, the DNA-binding domain of the gRNA comprises the nucleic acid sequence of SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, or SEQ ID NO: 126, or a biologically active fragment thereof. In certain embodiments, one or more of the first, second and third DNA molecules encoding the gRNA comprises the nucleic acid sequence of SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 59, SEQ ID NO: 60, SEQ ID NO: 61, SEQ ID NO: 62, SEQ ID NO: 63, SEQ ID NO: 84 or SEQ ID NO: 113, or a biologically active fragment thereof. In certain embodiments, the polynucleotide is at least 5 kb.

In certain embodiments, the first promoter, the second promoter, and the third promoter are pol III promoters. In certain embodiments, the first promoter, the second promoter, and the third promoter are selected from the group consisting of U6, H1 and 7SK. In certain embodiments, the first promoter is U6, the second promoter is H1, and the third promoter is 7SK. In certain embodiments, the polynucleotide further comprises a DNA molecule that encodes a Clustered Regularly Interspaced Short Palindromic Repeat (CRISPR)-associated endonuclease protein or a fragment thereof. In certain embodiments, the CRISPR-associated endonuclease is a class 2 CRISPR-associated endonuclease. In certain embodiments, the class 2 CRISPR-associated endonuclease is Cas9 or Cas12a. In certain embodiments, the DNA molecule that encodes the CRISPR-associated endonuclease or fragment thereof is operably linked to a tissue-specific promoter or a constitutive promoter. In certain embodiments, the tissue-specific promoter is a cancer tissue specific promoter. In certain embodiments, the cancer tissue-specific promoter is a human telomerase reverse transcriptase (hTERT) promoter. In certain embodiments, the tissue-specific promoter is a lung tissue-specific promoter. In certain embodiments, the constitutive promoter is a CMV promoter or a chicken beta-actin promoter (CAG) promoter. In certain embodiments, the DNA molecule that encodes the CRISPR-associated endonuclease is operably linked to at least one nucleus localization signal (NLS). In certain embodiments, the adenovirus is serotype 5. In certain embodiments, the adenovirus is replication incompetent. In certain embodiments, the adenovirus is replication competent.

In certain aspects, the disclosure relates to pharmaceutical composition comprising an adenovirus as described herein, and a pharmaceutically acceptable carrier.

In certain aspects, the disclosure relates to a pharmaceutical composition comprising: a) a first adenovirus comprising a DNA molecule encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in a gene; b) a second adenovirus comprising a DNA molecule encoding a Clustered Regularly Interspaced Short Palindromic Repeat (CRISPR)-associated endonuclease protein or a fragment thereof; and c) a pharmaceutically acceptable carrier, wherein the first adenovirus does not comprise a DNA molecule encoding a CRISPR-associated endonuclease protein or fragment thereof, and the second adenovirus does not comprise a DNA molecule encoding a gRNA.

In certain embodiments, the gene is selected from the group consisting of NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7, BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNXIT1, DNMT3A, SMCIA, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST, TSC2, EEF1A1, WHSC1, APOB, NUP133, AXIN1, PHF6, TET2, WT1, FLT3, FLT4, SMC3, CEBPA, RAD21, RAD50, RAD51, PTPDC1, ASXL1, EZH2, NPM1, SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA, LATS1, POLRMT, CDK4, COL5A1, PPP6C, MECOM, DACH1, MAP2K1, MAP2K2, RQCD1, DDX3X, NUP93, PPMID, CHD2, CHD3, CCND1, CCND2, CCND3, ACVR1, KMT2A, KMT2B, KMT2C, KMT2D, SIN3A, SCAF4, DICER1, FOXA2, CTNND1, MYC, MYCL, MYCN, SOX17, ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF2I, RIT1, MGA, ABL1, BRAF, CHEK1, FANCC, JAK2, MITF, PDCDILG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSFIR, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKBIA, PRDM1, DOT1L, FGFR1, LMO1, NKX2-1, PREX2, HSD3B1, LRP1B, TSHR, ATRX, CDC73, HSP90AA1, PRKCI, AURKA, PRKDC, VEGFA, AURKB, CDK12, FH, MAGI2, PRSS8, SMO, FLCN, IGFIR, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B, PARK2, RANBP2, SRC, IRS2, PAX5, RARA, BLM, FANCA, JAK1, FCRL4, LIG4, MAR, PWWP3A, MUC16, MUC17, FCGBP, FAT17, MMSET, IRTA2, TTN, DST, and STAT3. In certain embodiments, the gene is an NRF2 gene.

In certain aspects, the disclosure relates to a method of reducing expression or activity of a gene in a cancer cell comprising contacting the cancer cell with an adenovirus as described herein, or a pharmaceutical composition comprising an adenovirus as described herein, wherein the gRNA hybridizes to the gene and a CRISPR-associated endonuclease cleaves the gene, and wherein expression or activity of the gene is reduced in the cancer cell relative to a cancer cell in which the adenovirus is not introduced.

In certain aspects, the disclosure relates to a method of reducing expression or activity of a gene in a cancer cell in a subject, the method comprising administering to the subject an effective amount of a pharmaceutical composition as described herein to the subject, wherein the gRNA hybridizes to the gene and a CRISPR-associated endonuclease cleaves the gene, and wherein expression or activity of the gene is reduced in the cancer cell in the subject relative to a cancer cell in a subject that is not administered the pharmaceutical composition. In certain embodiments, expression of at least one allele of the gene is reduced in the cancer cell. In certain embodiments, expression of all alleles of the gene is reduced in the cancer cell. In certain embodiments, activity of the gene is reduced in the cancer cell. In certain embodiments, expression or activity of the gene is not completely eliminated in the cancer cell. In certain embodiments, expression or activity of the gene is completely eliminated in the cancer cell.

In certain aspects, the disclosure relates to a method of treating cancer in a subject, the method comprising administering to the subject a therapeutically effective amount of a pharmaceutical composition as described herein to the subject.

In certain aspects, the disclosure relates to a method of reducing resistance to one or more chemotherapeutic agents in a cancer in a subject in need thereof, the method comprising administering to the subject a therapeutically effective amount of a pharmaceutical composition as described herein to the subject. In certain embodiments, the cancer is selected from the group consisting of lung cancer, pancreatic cancer, melanoma, esophageal squamous cancer (ESC), head and neck squamous cell carcinoma (HNSCC), and breast cancer. In certain embodiments, the cancer is lung cancer. In certain embodiments, the lung cancer is non-small-cell lung cancer (NSCLC). In certain embodiments, the NSCLC is squamous-cell lung carcinoma. In certain embodiments, expression or activity of a wild-type allele of the gene in a non-cancerous cell of the subject is unaffected by administration of the pharmaceutical composition. In certain embodiments, the cancer is resistant to one or more chemotherapeutic agents. In certain embodiments, the method further comprises administering one or more chemotherapeutic agents to the subject. In certain embodiments, the one or more chemotherapeutic agents are selected from the group consisting of cisplatin, vinorelbine, carboplatin, paclitaxel, docetaxel, cabazitaxel and a combination thereof. In certain embodiments, the pharmaceutical composition is administered in an amount sufficient to reduce proliferation of cells of the cancer relative to cancer cells that are not treated with the pharmaceutical composition. In certain embodiments, the pharmaceutical composition is administered in an amount sufficient to reduce tumor growth relative to a tumor that is not treated with the pharmaceutical composition. In certain embodiments, the pharmaceutical composition is administered in an amount sufficient to reduce proliferation of cells of the cancer relative to cancer cells that are treated with the at least one chemotherapeutic agent but are not treated with the pharmaceutical composition. In certain embodiments, the pharmaceutical composition is administered in an amount sufficient to reduce tumor growth relative to a tumor that is treated with the at least one chemotherapeutic agent but is not treated with the pharmaceutical composition. In certain embodiments, the pharmaceutical composition is administered intratumorally. In certain embodiments, the subject is human.

In certain embodiments, the gene is selected from the group consisting of NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7, BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNXIT1, DNMT3A, SMCIA, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST, TSC2, EEF1A1, WHSC1, APOB, NUP133, AXIN1, PHF6, TET2, WT1, FLT3, FLT4, SMC3, CEBPA, RAD21, RAD50, RAD51, PTPDC1, ASXL1, EZH2, NPM1, SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA, LATS1, POLRMT, CDK4, COL5A1, PPP6C, MECOM, DACH1, MAP2K1, MAP2K2, RQCD1, DDX3X, NUP93, PPM1D, CHD2, CHD3, CCND1, CCND2, CCND3, ACVR1, KMT2A, KMT2B, KMT2C, KMT2D, SIN3A, SCAF4, DICER1, FOXA2, CTNND1, MYC, MYCL, MYCN, SOX17, ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF2I, RIT1, MGA, ABL1, BRAF, CHEK1, FANCC, JAK2, MITF, PDCDILG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSFIR, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKBIA, PRDM1, DOT1L, FGFR1, LMO1, NKX2-1, PREX2, HSD3B1, LRP1B, TSHR, ATRX, CDC73, HSP90AA1, PRKCI, AURKA, PRKDC, VEGFA, AURKB, CDK12, FH, MAGI2, PRSS8, SMO, FLCN, IGFIR, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B, PARK2, RANBP2, SRC, IRS2, PAX5, RARA, BLM, FANCA, JAK1, FCRL4, LIG4, MAR, PWWP3A, MUC16, MUC17, FCGBP, FAT17, MMSET, IRTA2, TTN, DST, and STAT3. In certain embodiments, the gene is an NRF2 gene.

### BRIEF DESCRIPTION OF THE DRAWINGS

FIG. 1 shows in vitro AAV serotype assessment of AAV5, AAV6, AAV6.2 and AAV9 using a scAAV-GFP construct. The transduction efficiencies for the 48 hour timepoint are on the left.

FIG. 2 shows in vitro AAV tropism assessment for AAV5 and AAV6 in various cell types. H1703: Squamous lung carcinoma; H520: Squamous lung carcinoma; A549: Adeno lung carcinoma; MRC5: Healthy lung fibroblast; HepG2: Liver hepatocellular carcinoma.

FIG. 3 shows exemplary sgRNA expression cassettes. The loop region is an inverted repeat.

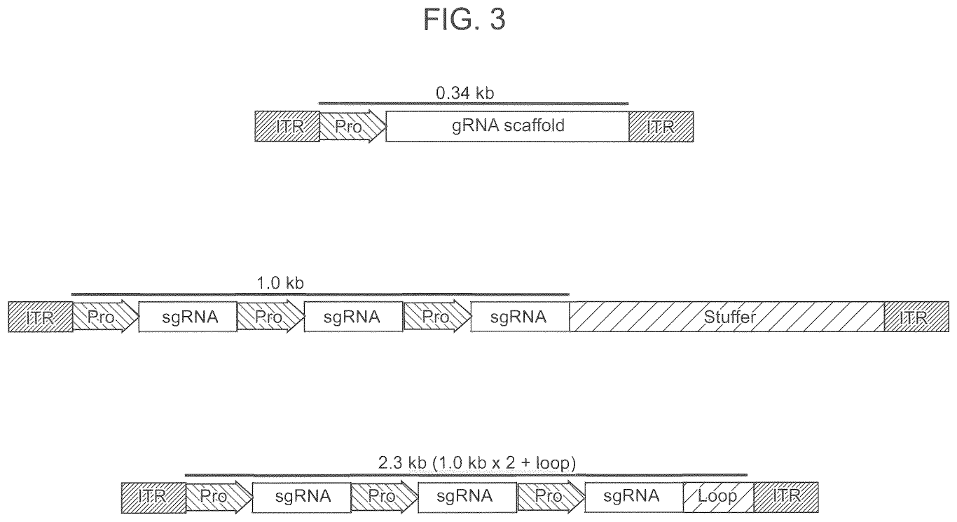

FIG. 4 shows in vivo AAV tropism assessment for AAV5 and AAV6 containing a luciferase gene in mice implanted with H1703 squamous non-small cell lung carcinoma cells. The representative median animal was chosen as the one with a flux value closest to the median of all the surviving animals at the last time point at which at least 50% of the animals remain in the group.

FIG. 5 shows tumor volume (mm³) and in vivo bioluminescence (Flux, pho/sec) over time for a representative mouse implanted with H1703 squamous non-small cell lung carcinoma cells and treated intratumorally with AAV6 containing a luciferase gene.

FIG. 6 shows the biodistribution of AAV6 containing a luciferase gene (AAV6-fLuc) injected into tumors of mice implanted with H1703 squamous non-small cell lung carcinoma cells. Ex-vivo bioluminescence was measured at Day 21. Animals were implanted subcutaneously with 5×10⁶ H1703 cells. After tumor volume reached >60 mm³, AAV6-fLuc was injected into the tumor directly and luciferase expression was observed by ex vivo imaging of isolated tissues. Data are shown unscaled.

FIG. 7 shows a comparison of the transduction efficacy in lung cancer cells of AAV and adenovirus as determined by GFP expression. The numbers right above the scale bars represented the percentage of GFP positive cells, and the numbers at the left corners of each image indicated the mean fluorescent intensity (MFI) of GFP.

FIG. 8 shows a comparison of AAV6 and adenovirus for expression of Cas9 in lung cancer cells, as indicated by editing efficacy of an NRF2 R34G gRNA.

FIG. 9 shows exemplary NRF2 R34G sgRNA adenoviral vector constructs for evaluation of different sgRNA copy numbers and dual vector systems.

FIG. 10 shows the % indel formation after adenoviral vector mediated gene editing in lung cancer cell lines C26-8 and C44-25 treated with NRF2 R34G sgRNAs.

FIG. 11 shows exemplary NRF2 R34G sgRNA adenoviral vector constructs for evaluation of cis and trans orientation of the sgRNA cassette relative to a spCas9 cassette comprising a CAG promoter.

FIG. 12 shows reduced Nrf2 protein expression levels in lung cancer cell lines treated with NRF2 R34G sgRNA adenoviral vectors.

FIG. 13 shows intratumoral delivery of adenoviral vector (fLuc) in H1703 44-25 subcutaneous xenograft model. Mice were intratumorally injected with Ad-CMV-fLuc and imaged 1-16 days post injection. The bioluminescent signal from each tumor was quantified by establishing a ROI and each value listed is total flux photons. The scale bars are representative for each image to the left of the bar.

FIG. 14 shows a schematic diagram of CRISPR/Cas9 adenoviral vectors targeting NRF2. The diagram depicts the U6 promoter driving R34G-targeting sgRNA expression followed by the chicken beta actin (CAG) promoter driving enhanced SpCas9 expression.

FIG. 15 shows representative images of Cas9 immunostaining of H1703 44-25-derived tumors. The two left hand panels depict two magnifications, 5× and 20×, from the same tumor section of an adenovirus-treated tumor. The scale bars represent 200 μm and 50 μm, respectively. The two right hand panels depict two magnifications, 2.5× and 20×, from the same tumor section of an untreated tumor. The scale bars represent 500 μm and 50 μm, respectively.

FIG. 16 shows representative images of Cas9 immunostaining of PDX-derived tumors. The two left hand panels depict two magnifications, 5× and 20×, from the same tumor section of an adenovirus-treated tumor. The scale bars represent 200 μm and 50 μm, respectively. The two right hand panels depict two magnifications, 2.5× and 20×, from the same tumor section of an untreated tumor. The scale bars represent 500 μm and 50 μm, respectively.

FIG. 17 shows a schematic diagram of CRISPR/Cas9 adenoviral vectors targeting NRF2. A) The diagram depicts the U6, H1 and 7SK promoters driving R34G-targeting sgRNA (SEQ ID 01) expression followed by the chicken beta actin (CAG) promoter driving enhanced SpCas9 expression. B) The diagram depicts the U6, H1 and 7SK promoters driving scrambled-targeting sgRNA (SEQ ID 02) expression followed by the chicken beta actin (CAG) promoter driving enhanced SpCas9 expression.

FIG. 18 shows delivery of CRISPR/Cas9 adenovirus in combination with chemotherapy inhibits tumor growth in a xenograft model. The graph shows tumor growth in NRF2-targeted or scramble-targeted mice over the course of 22 days. Mice were intratumorally injected on days 0, 2, and 7 with 3.6e9 pfu of either Ad-U6H17SK-R34G-CAG-eSpCas9 or U6H17SK-scramble-CAG-eSpCas9, as denoted by \hat{ }along the x axis. On days 3 and 10, all mice received 12.5 mg/kg of carboplatin and 5 mg/kg paclitaxel, intravenously, as denoted by * along the x axis.

FIGS. 19A-19C show genomic analyses of NRF2-targeted tumor tissues from xenograft mouse models. A) A single injection of 3.6e9 pfu of Ad-U6-R34G-CAG-eSpCas9 was intratumorally delivered to a patient-derived xenograft model. B) Two injections of 3.6e9 pfu of Ad-U6H17SK-R34G-CAG-eSpCas9 was intratumorally delivered to a H1703 44-25 xenograft model. C) Three injections of 3.6e9 pfu of Ad-U6H17SK-R34G-CAG-eSpCas9 was intratumorally delivered to a H1703 44-25 xenograft model. Tumor tissues from respective mouse models were excised and analyzed by PCR and Sanger sequencing.

FIGS. 20A-20E show a comparison of the CMV and CAG promoters fro driving Cas9 expression in cancer cell lines. Three heterozygous (PDC, 64-64, 64-17), panel A-C, and 2 homozygous (44-25, 44-58), panel D-E, NRF2 R34G genomic backgrounds were transduced with pAD-U6sgRNAR34G-CMVCas9 or pAD-U6sgRNAR34G-CAGCas9 at increasing MOI (0, 1, 10, 20, 100, 500) for 72 hrs. Cells were then collected for genomic DNA isolation and Sanger sequencing was performed to determine gene editing efficiency through DECODR. The bar graphs represent the INDEL %.

FIGS. 21A-21C show pAD-CMV-GFP Adenovirus 5 (Ad5) transduction efficiency testing in cancer cell lines and normal human primary cell lines. Cells were seeded in complete media at 1×10⁵ per well into a 24-well plate 24 hrs prior to viral exposure. The next day increasing MOI (0, 1, 10, 20, 100, 500) of Ad5 virus pAD-CMV-GFP was added to cells and further incubated for 72 hrs at this time eGFP expression was measured by Flow cytometry. Three categories of cells were tested: stable established cancer cell lines (A), Lung squamous cancer NRF2 R34G engineered cell lines (B), and primary cells (C). The bar graph represents the % of eGFP expressed.

FIG. 22 shows indel formation in two engineered lung cancer cell lines (e.g., the C26-8 and C44-25 cell lines) as analyzed by DECODR software to determine gene editing activity for each construct.

### DETAILED DESCRIPTION

In certain aspects, the disclosure relates to an adenovirus comprising a polynucleotide, wherein the polynucleotide comprises: (a) a first DNA molecule encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in a gene; and (b) a first promoter that is operably linked to the DNA molecule.

In some embodiments, the guide RNA is complementary to a target sequence in a gene selected from the group consisting of NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7, BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNXIT1, DNMT3A, SMCIA, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST. TSC2, EEF1A1, WHSC1, APOB, NUP133, AXIN1, PHF6. TET2, WT1, FLT3, FLT4, SMC3, CEBPA, RAD21, RAD50, RAD51, PTPDC1, ASXL1, EZH2, NPM1. SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA. LATS1, POLRMT, CDK4, COL5A1, PPP6C, MECOM, DACH1, MAP2K1, MAP2K2, RQCD1, DDX3X, NUP93, PPMID, CHD2, CHD3, CCND1, CCND2, CCND3, ACVR1, KMT2A, KMT2B, KMT2C, KMT2D, SIN3A, SCAF4, DICER1, FOXA2, CTNND1, MYC, MYCL, MYCN, SOX17. ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF21, RIT1, MGA, ABL1, BRAF, CHEK1, FANCC, JAK2, MITF, PDCDILG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSFIR, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKBIA, PRDM1, DOT1L, FGFR1, LMO1, NKX2-1, PREX2, HSD3B1, LRP1B. TSHR, ATRX, CDC73, HSP90AA1, PRKCI, AURKA, PRKDC, VEGFA, AURKB, CDK12, FH, MAGI2, PRSS8, SMO, FLCN, IGFIR, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B, PARK2, RANBP2, SRC, IRS2, PAX5, RARA, BLM, FANCA, JAK1, FCRL4, LIG4, MAR, PWWP3A, MUC16, MUC17, FCGBP. FAT17, MMSET, IRTA2, TTN, DST, and STAT3. In some embodiments, the guide RNA is complementary to a target sequence in a cancer driver gene, a disease-causing gene, an oncogene, or a variant tumor suppressor gene, such as, for example, one of the genes listed above.

In some embodiments, the guide RNA is complementary to a target sequence in an NRF2 gene. In some embodiments, the guide RNA is complementary to a variant NRF2 gene that is found only in cancer cells and not in wild-type NRF2 genes in normal (i.e., non-cancerous) cells (e.g. exon 1, 2, 3, 4, or 5). In some embodiments, the guide RNA is complementary to a sequence in exon 2 of a variant NRF2 gene that is found only in cancer cells.

### Definitions

Applicants specifically incorporate the entire contents of all cited references in this disclosure. Further, when an amount, concentration, or other value or parameter is given as either a range or a list of upper values and lower values, this is to be understood as specifically disclosing all ranges formed from any pair of any upper range limit or value and any lower range limit or value, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range.

The indefinite articles “a” and “an”, as used herein in the specification and in the claims, unless clearly indicated to the contrary, should be understood to mean “at least one”.

The phrase “and/or”, as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or, when used in the claims, “consisting of”, will refer to the inclusion of exactly one element of a number or list of elements. In general, the term “or” as used herein shall only be interpreted as indicating exclusive alternatives (i.e., “one or the other but not both”) when preceded by terms of exclusivity, “either”, “one of”, “only one of”, “exactly one of”. “Consisting essentially of”, when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “biologically active fragment” as used herein refers to a portion of a polynucleotide or polypeptide that retains at least one activity of the full length polynucleotide or polypeptide. For example, in some embodiments, a biologically active fragment of a promoter is a portion of the promoter that retains promoter activity. In some embodiments, a biologically active fragment of a DNA binding domain is a portion of the DNA binding domain that retains DNA binding activity. In some embodiments, a biologically active fragment of a DNA sequence encoding a guide RNA is a portion of the DNA sequence that encodes a functional guide RNA.

A “Clustered Regularly Interspaced Short Palindromic Repeat (CRISPR)-associated endonuclease protein-binding domain” or “Cas binding domain” refers to a nucleic acid element or domain within a nucleic acid sequence or polynucleotide sequence that, in an effective amount, will bind or have an affinity for one or a plurality of CRISPR-associated endonuclease (or functional fragments thereof). In some embodiments, in the presence of the one or a plurality of proteins (or functional fragments thereof) and a target sequence, the one or plurality of proteins and the nucleic acid element forms a biologically active CRISPR complex and/or can be enzymatically active on a target sequence. In some embodiments, the CRISPR-associated endonuclease is a class 1 or class 2 CRISPR-associated endonuclease, and in some embodiments, a Cas9 or Cas12a endonuclease. In a particular embodiment, the CRISPR-associated endonuclease is Cas9. The Cas9 endonuclease can have a nucleotide sequence identical to the wild type Streptococcus pyogenes sequence. In some embodiments, the CRISPR-associated endonuclease protein-binding domain is a Cas9 protein binding domain. In some embodiments, the DNA sequence encoding the Cas9 protein binding domain comprises or consists of the nucleic acid sequence of SEQ ID NO: 113, or a DNA sequence having at least 80%, 85%, 90%, 95%, 96%, 97%, 98% or 99% sequence identity to the nucleic acid sequence of SEQ ID NO: 113, or a biologically active fragment thereof.

In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Such species include: Acidovorax avenue, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., and Verminephrobacter eiseniae (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas9 endonucleases). In some embodiments, the CRISPR-associated endonuclease can be a Cas12a nuclease. The Cas12a nuclease can have a nucleotide sequence identical to a wild type Prevotella or Francisella sequence (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas12 endonucleases).

In some embodiments, the terms “(CRISPR)-associated endonuclease protein-binding domain” or “Cas binding domain” refer to a nucleic acid element or domain (e.g. and RNA element or domain) within a nucleic acid sequence that, in an effective amount, will bind to or have an affinity for one or a plurality of CRISPR-associated endonucleases (or functional fragments or variants thereof that are at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to a CRISPR-associated endonuclease). In some embodiments, the Cas binding domain consists of at least or no more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially associates or binds to a biologically active CRISPR-associated endonuclease at a concentration and within a microenvironment suitable for CRISPR system formation.

The “Clustered Regularly Interspaced Short Palindromic Repeats (CRISPR)—CRISPR associated (Cas) (CRISPR-Cas) system guide RNA” or “CRISPR-Cas system guide RNA” may comprise a transcription terminator domain. The term “transcription terminator domain” refers to a nucleic acid element or domain within a nucleic acid sequence (or polynucleotide sequence) that, in an effective amount, prevents bacterial transcription when the CRISPR complex is in a bacterial species and/or creates a secondary structure that stabilizes the association of the nucleic acid sequence to one or a plurality of Cas proteins (or functional fragments thereof) such that, in the presence of the one or a plurality of proteins (or functional fragments thereof), the one or plurality of Cas proteins and the nucleic acid element forms a biologically active CRISPR complex and/or can be enzymatically active on a target sequence in the presence of such a target sequence and a DNA-binding domain. In some embodiments, the transcription terminator domain consists of at least or no more than about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially drives association of the nucleic acid sequence (sgRNA, crRNA with tracrRNA, or other nucleic acid sequence) to a biologically active CRISPR complex at a concentration and microenvironment suitable for CRISPR complex formation.

The term “DNA-binding domain” refers to a nucleic acid element or domain within a nucleic acid sequence (e.g. a guide RNA) that is complementary to a target sequence (e.g. an NRF2 gene). In some embodiments, the DNA-binding domain will bind or have an affinity for an NRF2 gene such that, in the presence of a biologically active CRISPR complex, one or plurality of Cas proteins can be enzymatically active on the target sequence. In some embodiments, the DNA binding domain comprises at least one sequence that is capable of forming Watson Crick basepairs with a target sequence as part of a biologically active CRISPR system at a concentration and microenvironment suitable for CRISPR system formation.

“CRISPR system” refers collectively to transcripts or synthetically produced transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex. “target sequence” refers to a nucleic acid sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, the target sequence is a DNA polynucleotide and is referred to a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within an microenvironment suitable for association of such a system. In some embodiments, the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is an guanine and N is any naturally occurring nucleic acid. In some embodiments the target DNA comprises any one or combination of NNG, NNA, GAA, NNAGAAW and NGGNG, where G is an guanine, A is adenine, and N is any naturally occurring nucleic acid

In some embodiments, a target sequence is located in the nucleus or cytoplasm of a cell.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional (bind the Cas protein or functional fragment thereof). In some embodiments, the tracr sequence has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of sequence complementarity along the length of the tracr mate sequence when optimally aligned. In some embodiments, one or more vectors driving expression of one or more elements of a CRISPR system are introduced into a host cell such that the presence and/or expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. With at least some of the modification contemplated by this disclosure, in some embodiments, the guide sequence or RNA or DNA sequences that form a CRISPR complex are at least partially synthetic. The CRISPR system elements that are combined in a single vector may be arranged in any suitable orientation, such as one element located 5′ with respect to (“upstream” of) or 3′ with respect to (“downstream” of) a second element. In some embodiments, the disclosure relates to a composition comprising a chemically synthesized guide sequence. In some embodiments, the chemically synthesized guide sequence is used in conjunction with a vector comprising a coding sequence that encodes a CRISPR enzyme, such as a class 2 Cas9 or Cas12a protein. In some embodiments, the chemically synthesized guide sequence is used in conjunction with one or more vectors, wherein each vector comprises a coding sequence that encodes a CRISPR enzyme, such as a class 2 Cas9 or Cas12a protein. The coding sequence of one element may be located on the same or opposite strand of the coding sequence of a second element, and oriented in the same or opposite direction. In some embodiments, a single promoter drives expression of a transcript encoding a CRISPR enzyme and one or more additional (second, third, fourth, etc.) guide sequences, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, one or more additional guide sequence, tracr mate sequence, and tracr sequence are each a component of different nucleic acid sequences. For instance, in the case of a tracr and tracr mate sequences and in some embodiments, the disclosure relates to a composition comprising at least a first and second nucleic acid sequence, wherein the first nucleic acid sequence comprises a tracr sequence and the second nucleic acid sequence comprises a tracr mate sequence, wherein the first nucleic acid sequence is at least partially complementary to the second nucleic acid sequence such that the first and second nucleic acid for a duplex and wherein the first nucleic acid and the second nucleic acid either individually or collectively comprise a DNA-targeting domain, a Cas protein binding domain, and a transcription terminator domain. In some embodiments, the CRISPR enzyme, one or more additional guide sequence, tracr mate sequence, and tracr sequence are operably linked to and expressed from the same promoter. In some embodiments, the disclosure relates to compositions comprising any one or combination of the disclosed domains on one guide sequence or two separate tracrRNA/crRNA sequences with or without any of the disclosed modifications. Any methods disclosed herein also relate to the use of tracrRNA/crRNA sequence interchangeably with the use of a guide sequence, such that a composition may comprise a single synthetic guide sequence and/or a synthetic tracrRNA/crRNA with any one or combination of modified domains disclosed herein.

In some embodiments, a guide RNA can be a short, synthetic, chimeric tracrRNA/crRNA (a “single-guide RNA” or “sgRNA”). A guide RNA may also comprise two short, synthetic tracrRNA/crRNAs (a “dual-guide RNA” or ‘dgRNA”).

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features.

As used herein, “cancer” refers to all types of cancer or neoplasm or malignant tumors found in humans, including, but not limited to: leukemias, lymphomas, melanomas, carcinomas and sarcomas. As used herein, the terms or language “cancer,” “neoplasm,” and “tumor.” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells. In certain embodiments, the cancer is a blood tumor (i.e., a non-solid tumor). In some embodiments, the cancer is lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, or breast invasive carcinoma (see, e.g., Kerins et al., Sci. Rep. 8:12846 (2018)).

In certain embodiments, the cancer is a solid tumor. A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

Specific criteria for the staging of cancer are dependent on the specific cancer type based on tumor size, histological characteristics, tumor markers, and other criteria known by those of skill in the art. Generally, cancer stages can be described as follows:

### Stage 0—Carcinoma In Situ

Stage I. Stage II, and Stage III-Higher numbers indicate more extensive disease: Larger tumor size and/or spread of the cancer beyond the organ in which it first developed to nearby lymph nodes and/or tissues or organs adjacent to the location of the primary tumor

### Stage IV—the Cancer has Spread to Distant Tissues or Organs

As used herein, a “variant”, “mutant”, or “mutated” polynucleotide contains at least one polynucleotide sequence alteration as compared to the polynucleotide sequence of the corresponding wild-type or parent polynucleotide. Mutations may be natural, deliberate, or accidental. Mutations include substitutions, deletions, and insertions.

As used herein, the terms “treat,” “treating” or “treatment” refer to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, a decrease in pain or an increase in the QOL as judged by a qualified individual, e.g., a treating physician, e.g., using accepted assessment tools of pain and QOL is not considered to be a “treatment” of the cancer.

“Chemotherapeutic agent” refers to a drug used for the treatment of cancer. Chemotherapeutic agents include, but are not limited to, small molecules, hormones and hormone analogs, and biologics (e.g., antibodies, peptide drugs, nucleic acid drugs). In certain embodiments, chemotherapy does not include hormones and hormone analogs.

A “cancer that is resistant to one or more chemotherapeutic agents” is a cancer that does not respond, or ceases to respond to treatment with a chemotherapeutic regimen, i.e., does not achieve at least stable disease (i.e., stable disease, partial response, or complete response) in the target lesion either during or after completion of the chemotherapeutic regimen. Resistance to one or more chemotherapeutic agents results in, e.g., tumor growth, increased tumor burden, and/or tumor metastasis.

A “therapeutically effective amount” is that amount sufficient, at dosages and for periods of time necessary, to achieve a desired therapeutic result, such as for treatment of a disease (e.g. cancer), condition, or disorder, and/or pharmacokinetic or pharmacodynamic effect of the treatment in a subject. A therapeutically effective amount can be administered in one or more administrations. The therapeutically effective amount may vary according to factors such as the disease state, age, sex, and weight of the subject.

As used herein, a “vector” is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., an adenoviruses). Viral vectors also include polynucleotides carried by a virus for transfection into a host cell. Recombinant expression vectors can comprise a nucleic acid suitable for expression of the nucleic acid in a host cell, which means that the recombinant expression vectors include one or more regulatory elements (e.g. promoters), which may be selected on the basis of the host cells to be used for expression, that is operatively-linked to the nucleic acid sequence to be expressed.

The term “promoter” as used herein refers to a DNA regulatory element that drives expression of a polynucleotide. Promoters include those that direct constitutive expression of a polynucleotide sequence in many types of host cell and those that direct expression of the nucleotide sequence only in certain host cells (e.g., tissue-specific promoter). A tissue-specific promoter may direct expression primarily in a desired tissue of interest, such as muscle, neuron, bone, skin, blood, specific organs (e.g. lung, liver, pancreas), or particular cell types (e.g. lymphocytes). In some embodiments, the tissue-specific promoter is a cancer-specific promoter, i.e., a promoter that directs expression primarily in cancer cells. Promoters may also direct expression in a temporal-dependent manner, such as in a cell-cycle dependent or developmental stage-dependent manner, which may or may not also be tissue or cell-type specific.

The term “expression cassette” as used herein refers to a polynucleotide comprising a nucleic acid sequence encoding a polypeptide or RNA molecule, wherein the nucleic acid sequence is operably linked to promoter. In some embodiments, the expression cassette comprises one or more nucleic acid sequences encoding a gRNA. In some embodiments, the expression cassette comprises the nucleic acid sequence of SEQ ID NO: 125, or a nucleic acid sequence having at least 80%, 85%, 90%, 95%, 96%, 97%, 98% or 99% sequence identity to SEQ ID NO: 125, or a biologically active fragment thereof. SEQ ID NO: 125 comprises three repeats of a nucleic acid sequence (SEQ ID NO: 124) encoding the gRNA of SEQ ID NO: 128. Each repeat is operably linked to the H1, U6 or 7SK promoter.

The terms “polynucleotide” and “nucleic acid sequence” are used interchangeably herein.

### Target Genes

In some embodiments, the gene targeted by the gRNA is NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7, BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNXIT1, DNMT3A, SMCIA, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST, TSC2, EEF1A1, WHSC1, APOB, NUP133, AXIN1, PHF6, TET2, WT1, FLT3, FLT4, SMC3, CEBPA, RAD21, RAD50, RAD51, PTPDC1, ASXL1, EZH2, NPM1, SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA, LATS1, POLRMT, CDK4, COL5A1, PPP6C, MECOM, DACH1, MAP2K1, MAP2K2, RQCD1, DDX3X, NUP93, PPMID, CHD2, CHD3, CCND1, CCND2, CCND3, ACVR1. KMT2A, KMT2B, KMT2C, KMT2D, SIN3A, SCAF4, DICER1, FOXA2, CTNND1, MYC, MYCL, MYCN, SOX17, ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF2I, RIT1, MGA, ABL1, BRAF, CHEK1, FANCC, JAK2, MITF, PDCDILG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSFIR, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKB1A, PRDM1, DOT1L, FGFR1, LMO1, NKX2-1, PREX2, HSD3B1, LRP1B, TSHR, ATRX, CDC73, HSP90AA1, PRKCI, AURKA, PRKDC, VEGFA, AURKB, CDK12, FH, MAGI2, PRSS8, SMO, FLCN, IGFIR, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B. PARK2, RANBP2, SRC, IRS2, PAX5, RARA, BLM, FANCA, JAK1, FCRLA, LIG4, MAR, PWWP3A, MUC16, MUC17, FCGBP, FAT17, MMSET, IRTA2, TTN, DST, or STAT3.

### NRF2

In some embodiments, the gene targeted by the gRNA is Nuclear Factor Erythroid 2-Related Factor (NRF2). NRF2 is considered the master regulator of 100-200 target genes involved in cellular responses to oxidative/electrophilic stress. Targets include glutathione (GSH) mediators, antioxidants and genes controlling efflux pumps. Hayden, et al., Urol. Oncol. Semin. Orig. Investig. 32, 806-814 (2014). NRF2 is also known to regulate expression of genes involved in protein degradation and detoxification and is negatively regulated by Kelch-like ECH-associated protein 1 (KEAP1), a substrate adapter for the Cul3-dependent E3 ubiquitin ligase complex. Under normal conditions, Keap1 constantly targets NRF2 for ubiquitin-dependent degradation maintaining low expression of NRF2 on downstream target genes. However, chemotherapy has been shown to activate transcriptional activity of the NRF2 target genes often triggering a cytoprotective response; enhanced expression of NRF2 occurs in response to environmental stress or detrimental growth conditions. Other mechanisms that lead to NRF2 upregulation include mutations in KEAP1 or epigenetic changes of the promoter region. The upregulation of NRF2 expression leads to an enhanced resistance of cancer cells to chemotherapeutic drugs, which by their very action induce an unfavorable environment for cell proliferation. Indeed, Hayden et al. (ibid) have clearly demonstrated that increased NRF2 expression leads to the resistance of cancer cells to chemotherapeutic drugs including cisplatin. Singh et al. (2010, Antioxidants & Redox Signaling 13) also showed that constitutive expression of NRF2 leads to radioresistance, and inhibition of NRF2 causes increased endogenous reactive oxygen species (ROS) levels as well as decreased survival. Recently, Torrente et al. (Oncogene (2017). doi: 10.1038/onc.2017.221) identified crosstalk between NRF2 and the homeodomain interacting protein kinase two, HIPK2, demonstrating that HIPK2 exhibits a cytoprotective effect through NRF2.

By using CRISPR/Cas9, it is possible to target and knock out the mutated NRF2 protein causing chemoresistance, while not disrupting the function of wildtype NRF2 protein. Thus, some embodiments are directed to reducing or, in some embodiments, eliminating expression of variant NRF2s found only in cancer cells and not in non-cancerous cells. These variants are commonly found within the Neh2 Domain of NRF2, which is known as the KEAP1 binding domain. In some embodiments, the NRF2 mutations can be those found in Table 1 below.

TABLE 1

| | | Base | Notes/Amino |
|---|---|---|---|
| | | position | Acid substitution |
| SEQ | Sequence, with substitution | in | (relative to |
| ID | identified in | SEQ ID | position in SEQ |
| NO: | brackets | NO: 7 | ID NO:8) |
| 41 | TTTGATTGACATACTTTGGAGGC(C>G) | 205-227 | Q26E |
| 42 | CTTACTCCAAGATCTATATCTT(T>G)G | 249-227 | Reverse |
| | | | complement; |
| | | | Q26P |
| 43 | ATACTTTGGAGGCAAGATATAGA(A>G) | 215-237 | D29G |
| 44 | AGGCAAGATATAGATCTTGGAGT(T>G) | 224-246 | V32G |
| 45 | GATATAGATCTTGGAGTAAGTC(C>G)G | 230-252 | R34G |
| 46 | TGACTGAAGTCAAATACTTCTC(C>G)G | 273-251 | Reverse |
| | | | complement; |
| | | | R34P |
| 47 | TTCATCTAGTTGTAACTGAGCGA(A>G) | 385-363 | Reverse |
| | | | complement; |
| | | | F71S |
| 48 | ATTCACCTGTCTCTTCATCTAGT(T>G) | 398-376 | Reverse |
| | | | complement; |
| | | | Q75H |
| 49 | CTCAGTTACAACTAGATGAAGA(A>G)G | 366-388 | E79G |
| 50 | TGAATTGGGAGAAATTCACCTGT(T>G) | 411-389 | Reverse |
| | | | complement; |
| | | | T80P |
| 51 | CAACTAGATGAAGAGACAGGTGA(A>G) | 374-396 | E82W |
| 52 | ATAATAGCTCCTCCCAAACTTGC(C>G) | 728-706 | Reverse |
| | | | complement; |
| | | | E185D |
| 57 | TTTTTCGCTCAGTTACAACTAGA(A>G) | 360-381 | D77G |
| 58 | CAACTAGATGAAGAGACAGGTGA(A>G) | 374-396 | E82G |

PAM sequence underlined.

In some embodiments, a variant NRF2 polypeptide encoded by the variant NRF2 gene in a cancer cell can comprise one or more amino acid substitutions of: (a) Q26E, D29G, V32G, R34G, F71S, Q75H, D77G, E79G, T80P, E82W, or E185D relative to SEQ ID NO:8; (b) Q26E, D29G, V32G, R34P, F71S, Q75H, D77G, E79G, T80P, E82W, or E185D relative to SEQ ID NO: 8; (c) Q26P, D29G, V32G, R34G, F71S, Q75H, D77G, E79G, T80P, E82W, or E185D relative to SEQ ID NO:8; (d) Q26P, D29G, V32G, R34P, F71S, Q75H, D77G, E79G, T80P, E82W, or E185D relative to SEQ ID NO:8; (e) Q26E, D29G, V32G, R34G, F71S, Q75H, D77G, E79G, T80P, E82G, or E185D relative to SEQ ID NO:8; (f) Q26E, D29G, V32G, R34P, F71S, Q75H, D77G, E79G, T80P, E82G, or E185D relative to SEQ ID NO:8; (g) Q26P, D29G, V32G, R34G, F71S, Q75H, D77G, E79G, T80P, E82G, or E185D relative to SEQ ID NO:8; or (h) Q26P, D29G, V32G, R34P. F71S, Q75H, D77G, E79G, T80P, E82G, or E185D relative to SEQ ID NO: 8.

In some embodiments, a variant NRF2 gene can comprise: (a) one or more polynucleotide sequences selected from the group consisting of SEQ ID NO:41 substituting positions 205-227 of SEQ ID NO:7, SEQ ID NO:43 substituting positions 215-237 of SEQ ID NO:7, SEQ ID NO: 44 substituting positions 224-246 of SEQ ID NO:7. SEQ ID NO:45 substituting positions 230-252 of SEQ ID NO:7, SEQ ID NO:47 substituting positions 385-363 of the reverse complement of SEQ ID NO:7, SEQ ID NO:48 substituting positions 398-376 of the reverse complement of SEQ ID NO:7, SEQ ID NO:49 substituting positions 366-388 of SEQ ID NO:7, SEQ ID NO:50 substituting positions 411-389 of the reverse complement of SEQ ID NO:7, SEQ ID NO: 51 substituting positions 374-396 of SEQ ID NO:7, SEQ ID NO:52 substituting positions 728-706 of the reverse complement of SEQ ID NO:7, or SEQ ID NO:57 substituting positions 360-381 of SEQ ID NO:7; (b) one or more polynucleotide sequences selected from the group consisting of SEQ ID NO:41 substituting positions 205-227 of SEQ ID NO:7, SEQ ID NO:43 substituting positions 215-237 of SEQ ID NO:7, SEQ ID NO:44 substituting positions 224-246 of SEQ ID NO:7, SEQ ID NO:46 substituting positions 273-251 of the reverse complement of SEQ ID NO:7, SEQ ID NO:47 substituting positions 385-363 of the reverse complement of SEQ ID NO: 7, SEQ ID NO:48 substituting positions 398-376 of the reverse complement of SEQ ID NO: 7, SEQ ID NO:49 substituting positions 366-388 of SEQ ID NO:7, SEQ ID NO:50 substituting positions 411-389 of the reverse complement of SEQ ID NO:7, SEQ ID NO:51 substituting positions 374-396 of SEQ ID NO:7. SEQ ID NO:52 substituting positions 728-706 of the reverse complement of SEQ ID NO:7, or SEQ ID NO:57 substituting positions 360-381 of SEQ ID NO:7; (c) one or more polynucleotide sequences selected from the group consisting of SEQ ID NO:42 substituting positions 249-227 of the reverse complement of SEQ ID NO:7, SEQ ID NO: 43 substituting positions 215-237 of SEQ ID NO:7, SEQ ID NO:44 substituting positions 224-246 of SEQ ID NO:7. SEQ ID NO:45 substituting positions 230-252 of SEQ ID NO:7, SEQ ID NO: 47 substituting positions 385-363 of the reverse complement of SEQ ID NO:7, SEQ ID NO: 48 substituting positions 398-376 of the reverse complement of SEQ ID NO:7, SEQ ID NO: 49 substituting positions 366-388 of SEQ ID NO:7. SEQ ID NO:50 substituting positions 411-389 of the reverse complement of SEQ ID NO:7, SEQ ID NO:51 substituting positions 374-396 of SEQ ID NO:7, SEQ ID NO:52 substituting positions 728-706 of the reverse complement of SEQ ID NO:7, or SEQ ID NO:57 substituting positions 360-381 of SEQ ID NO:7; (d) one or more polynucleotide sequences selected from the group consisting of SEQ ID NO:42 substituting positions 249-227 of the reverse complement of SEQ ID NO:7, SEQ ID NO:43 substituting positions 215-237 of SEQ ID NO:7, SEQ ID NO:44 substituting positions 224-246 of SEQ ID NO: 7, SEQ ID NO:46 substituting positions 273-251 of the reverse complement of SEQ ID NO: 7, SEQ ID NO:47 substituting positions 385-363 of the reverse complement of SEQ ID NO: 7, SEQ ID NO:48 substituting positions 398-376 of the reverse complement of SEQ ID NO: 7, SEQ ID NO:49 substituting positions 366-388 of SEQ ID NO:7, SEQ ID NO:50 substituting positions 411-389 of the reverse complement of SEQ ID NO:7, SEQ ID NO:51 substituting positions 374-396 of SEQ ID NO:7. SEQ ID NO:52 substituting positions 728-706 of the reverse complement of SEQ ID NO:7, or SEQ ID NO:57 substituting positions 360-381 of SEQ ID NO:7; (e) one or more polynucleotide sequences selected from the group consisting of SEQ ID NO:41 substituting positions 205-227 of SEQ ID NO:7, SEQ ID NO:43 substituting positions 215-237 of SEQ ID NO:7, SEQ ID NO:44 substituting positions 224-246 of SEQ ID NO: 7, SEQ ID NO:45 substituting positions 230-252 of SEQ ID NO:7, SEQ ID NO:47 substituting positions 385-363 of the reverse complement of SEQ ID NO:7, SEQ ID NO:48 substituting positions 398-376 of the reverse complement of SEQ ID NO:7, SEQ ID NO:49 substituting positions 366-388 of SEQ ID NO:7, SEQ ID NO:50 substituting positions 411-389 of the reverse complement of SEQ ID NO:7, SEQ ID NO:58 substituting positions 374-396 of SEQ ID NO:7, SEQ ID NO:52 substituting positions 728-706 of the reverse complement of SEQ ID NO: 7, or SEQ ID NO:57 substituting positions 360-381 of SEQ ID NO:7; (f) one or more polynucleotide sequences selected from the group consisting of SEQ ID NO:41 substituting positions 205-227 of SEQ ID NO:7, SEQ ID NO:43 substituting positions 215-237 of SEQ ID NO: 7, SEQ ID NO:44 substituting positions 224-246 of SEQ ID NO:7, SEQ ID NO:46 substituting positions 273-251 of the reverse complement of SEQ ID NO:7, SEQ ID NO:47 substituting positions 385-363 of the reverse complement of SEQ ID NO:7, SEQ ID NO:48 substituting positions 398-376 of the reverse complement of SEQ ID NO:7, SEQ ID NO:49 substituting positions 366-388 of SEQ ID NO:7, SEQ ID NO:50 substituting positions 411-389 of the reverse complement of SEQ ID NO:7. SEQ ID NO:58 substituting positions 374-396 of SEQ ID NO:7. SEQ ID NO:52 substituting positions 728-706 of the reverse complement of SEQ ID NO: 7, or SEQ ID NO:57 substituting positions 360-381 of SEQ ID NO:7; (g) one or more polynucleotide sequences selected from the group consisting of SEQ ID NO:42 substituting positions 249-227 of the reverse complement of SEQ ID NO:7, SEQ ID NO:43 substituting positions 215-237 of SEQ ID NO:7, SEQ ID NO:44 substituting positions 224-246 of SEQ ID NO: 7. SEQ ID NO:45 substituting positions 230-252 of SEQ ID NO:7, SEQ ID NO:47 substituting positions 385-363 of the reverse complement of SEQ ID NO:7, SEQ ID NO:48 substituting positions 398-376 of the reverse complement of SEQ ID NO:7, SEQ ID NO:49 substituting positions 366-388 of SEQ ID NO:7. SEQ ID NO:50 substituting positions 411-389 of the reverse complement of SEQ ID NO:7, SEQ ID NO:58 substituting positions 374-396 of SEQ ID NO:7, SEQ ID NO:52 substituting positions 728-706 of the reverse complement of SEQ ID NO: 7, or SEQ ID NO:57 substituting positions 360-381 of SEQ ID NO:7; or (h) one or more polynucleotide sequences selected from the group consisting of SEQ ID NO:42 substituting positions 249-227 of the reverse complement of SEQ ID NO:7, SEQ ID NO:43 substituting positions 215-237 of SEQ ID NO:7, SEQ ID NO:44 substituting positions 224-246 of SEQ ID NO: 7, SEQ ID NO:46 substituting positions 273-251 of the reverse complement of SEQ ID NO: 7, SEQ ID NO:47 substituting positions 385-363 of the reverse complement of SEQ ID NO: 7, SEQ ID NO:48 substituting positions 398-376 of the reverse complement of SEQ ID NO: 7, SEQ ID NO:49 substituting positions 366-388 of SEQ ID NO:7, SEQ ID NO:50 substituting positions 411-389 of the reverse complement of SEQ ID NO:7, SEQ ID NO:58 substituting positions 374-396 of SEQ ID NO:7, SEQ ID NO:52 substituting positions 728-706 of the reverse complement of SEQ ID NO:7, or SEQ ID NO:57 substituting positions 360-381 of SEQ ID NO:7.

### EGFR

In some embodiments, the gene targeted by the gRNA is epidermal growth factor receptor (EGFR). EGFR is a transmembrane glycoprotein that is a member of the protein kinase superfamily. This protein is a receptor for members of the epidermal growth factor family. EGFR is a cell surface protein that binds to epidermal growth factor, thus inducing receptor dimerization and tyrosine autophosphorylation leading to cell proliferation. Mutations in this gene are associated with lung cancer. EGFR is a component of the cytokine storm which contributes to a severe form of COVID-19 resulting from infection with severe acute respiratory syndrome coronavirus-2 (SARS-COV-2). In some embodiments, a de novo PAM (CTG→CGG) is created in EGFR by a L858R mutation (highlighted in SEQ ID NOs: 129 and 130 below).

```
SEQ ID NO: 129
      1 agacgtccgg gcagcccccg gcgcagcgcg gccgcagcag cctccgcccc ccgcacggtg
    61 tgagcgcccg acgcggccga ggcggccgga gtcccgagct agccccggcg gccgccgccg
  121 cccagaccgg acgacaggcc acctcgtcgg cgtccgcccg agtccccgcc tcgccgccaa
  181 cgccacaacc accgcgcacg gccccctgac tccgtccagt attgatcggg agagccggag
  241 cgagctcttc ggggagcagc gatgcgaccc tccgggacgg ccggggcagc gctcctggcg
  301 ctgctggctg cgctctgccc ggcgagtcgg gctctggagg aaaagaaagt ttgccaaggc
  361 acgagtaaca agctcacgca gttgggcact tttgaagatc attttctcag cctccagagg
  421 atgttcaata actgtgaggt ggtccttggg aatttggaaa ttacctatgt gcagaggaat
  481 tatgatcttt ccttcttaaa gaccatccag gaggtggctg gttatgtcct cattgccctc
  541 aacacagtgg agcgaattcc tttggaaaac ctgcagatca tcagaggaaa tatgtactac
  601 gaaaattcct atgccttagc agtcttatct aactatgatg caaataaaac cggactgaag
  661 gagctgccca tgagaaattt acaggaaatc ctgcatggcg ccgtgcggtt cagcaacaac
  721 cctgccctgt gcaacgtgga gagcatccag tggcgggaca tagtcagcag tgactttctc
  781 agcaacatgt cgatggactt ccagaaccac ctgggcagct gccaaaagty tgatccaagc
  841 tgtcccaatg ggagctgctg gggtgcagga gaggagaact gccagaaact gaccaaaatc
  901 atctgtgccc agcagtgctc cgggcgctgc cgtggcaagt cccccagtga ctgctgccac
  961 aaccagtgtg ctgcaggctg cacaggcccc cgggagagcg actgcctggt ctgccgcaaa
1021 ttccgagacg aagccacgtg caaggacacc tgccccccac tcatgctcta caaccccacc
1081 acgtaccaga tggatgtgaa ccccgagggc aaatacagct ttggtgccac ctgcgtgaag
1141 aagtgtcccc gtaattatgt ggtgacagat cacggctcgt gcgtccgagc ctgtggggcc
1201 gacagctatg agatggagga agacggcgtc cgcaagtgta agaagtgcga agggccttgc
1261 cgcaaagtgt gtaacggaat aggtattggt gaatttaaag actcactctc cataaatgct
1321 acgaatatta aacacttcaa aaactgcacc tccatcagtg gcgatctcca catcctgccg
1381 gtggcattta ggggtgactc cttcacacat actcctcctc tggatccaca ggaactggat
1441 attctgaaaa ccgtaaagga aatcacaggg tttttgctga ttcaggcttg gcctgaaaac
1501 aggacggacc tccatgcctt tgagaaccta gaaatcatac gcggcaggac caagcaacat
1561 ggtcagtttt ctcttgcagt cgtcagcctg aacataacat ccttgggatt acgctccctc
1621 aaggagataa gtgatggaga tgtgataatt tcaggaaaca aaaatttgtg ctatgcaaat
1681 acaataaact ggaaaaaact gtttgggacc tccggtcaga aaaccaaaat tataagcaac
1741 agaggtgaaa acagctgcaa ggccacaggc caggtctgcc atgccttgtg ctcccccgag
1801 ggctgctggg gcccggagcc cagggactgc gtctcttgcc ggaatgtcag ccgaggcagg
1861 gaatgcgtgg acaagtgcaa ccttctggag ggtgagccaa gggagtttgt ggagaactct
1921 gagtgcatac agtgccaccc agagtgcctg cctcaggcca tgaacatcac ctgcacagga
1981 cggggaccag acaactgtat ccagtgtgcc cactacattg acggccccca ctgcgtcaag
2041 acctgcccgg caggagtcat gggagaaaac aacaccctgg tctggaagta cgcagacgcc
2101 ggccatgtgt gccacctgtg ccatccaaac tgcacctacg gatgcactgg gccaggtctt
2161 gaaggctgtc caacgaatgg gcctaagatc ccgtccatcg ccactgggat ggtgggggcc
2221 ctcctcttgc tgctggtggt ggccctgggg atcggcctct tcatgcgaag gcgccacatc
2281 gttcggaagc gcacgctgcg gaggctgctg caggagaggg agcttgtgga gcctcttaca
2341 cccagtggag aagctcccaa ccaagctctc ttgaggatct tgaaggaaac tgaattcaaa
2401 aagatcaaag tgctgggctc cggtgcgttc ggcacggtgt ataagggact ctggatccca
2461 gaaggtgaga aagttaaaat tcccgtcgct atcaaggaat taagagaagc aacatctccg
2521 aaagccaaca aggaaatcct cgatgaagcc tacgtgatgg ccagcgtgga caacccccac
2581 gtgtgccgcc tgctgggcat ctgcctcacc tccaccgtgc agctcatcac gcagctcatg
2641 cccttcggct gcctcctgga ctatgtccgg gaacacaaag acaatattgg ctcccagtac
2701 ctgctcaact ggtgtgtgca gatcgcaaag ggcatgaact acttggagga ccgtcgcttg
2761 gtgcaccgcg acctggcagc caggaacgta ctggtgaaaa caccgcagca tgtcaagatc
2821 acagattttg ggctggccaa actgctgggt gcggaagaga aagaatacca tgcagaagga
2881 ggcaaagtgc ctatcaagtg gatggcattg gaatcaattt tacacagaat ctatacccac
2941 cagagtgatg tctggagcta cggggtgact gtttgggagt tgatgacctt tggatccaag
3001 ccatatgacg gaatccctgc cagcgagatc tcctccatcc tggagaaagg agaacgcctc
3061 cctcagccac ccatatgtac catcgatgt tacatgatca tggtcaagtg ctggatgata
3121 gacgcagata gtcgcccaaa gttccgtgag ttgatcatcg aattctccaa aatggcccga
3181 gacccccagc gctaccttgt cattcagggg gatgaaagaa tgcatttgcc aagtcctaca
3241 gactccaact tctaccgtgc cctgatggat gaagaagaca tggacgacgt ggtggatgcc
3301 gacgagtacc tcatcccaca gcagggcttc ttcagcagcc cctccacgtc acggactccc
3361 ctcctgagct ctctgagtgc aaccagcaac aattccaccg tggcttgcat tgatagaaat
3421 gggctgcaaa gctgtcccat caaggaagac agcttcttgc agcgatacag ctcagacccc
3481 acaggcgcct tgactgagga cagcatagac gacaccttcc tcccagtgcc tgaatacata
3541 aaccagtccg ttcccaaaag gcccgctggc tctgtgcaga atcctgtcta tcacaatcag
3601 cctctgaacc ccgcgcccag cagagaccca cactaccagg acccccacag cactgcagtg
3661 ggcaaccccg agtatctcaa cactgtccag cccacctgtg tcaacagcac attcgacagc
3721 cctgcccact gggcccagaa aggcagccac caaattagcc tggacaaccc tgactaccag
3781 caggacttct ttcccaagga agccaagcca aatggcatct ttaagggctc cacagctgaa
3841 aatgcagaat acctaagggt cgcgccacaa agcagtgaat ttattggagc atgaccacgg
3901 aggatagtat gagccctaaa aatccagact ctttcgatac ccaggaccaa gccacagcag
3961 gtcctccatc ccaacagcca tgcccgcatt agctcttaga cccacagact ggttttgcaa
4021 cgtttacacc gactagccag gaagtacttc cacctcgggc acattttggg aagttgcatt
4081 cctttgtctt caaactgtga agcatttaca gaaacgcatc cagcaagaat attgtccctt
4141 tgagcagaaa tttatctttc aaagaggtat atttgaaaaa aaaaaaaagt atatgtgagg
4201 atttttattg attggggatc ttggagtttt tcattgtcgc tattgatttt tacttcaatg
4261 ggctcttcca acaaggaaga agcttgctgg tagcacttgc taccctgagt tcatccaggc
4321 ccaactgtga gcaaggagca caagccacaa gtcttccaga ggatgcttga ttccagtggt
4381 tctgcttcaa ggcttccact gcaaaacact aaagatccaa gaaggccttc atggccccag
4441 caggccggat cggtactgta tcaagtcatg gcaggtacag taggataagc cactctgtcc
4501 cttcctgggc aaagaagaaa cggaggggat ggaattcttc cttagactta cttttgtaaa
4561 aatgtcccca cggtacttac tccccactga tggaccagtg gtttccagtc atgagcgtta
4621 gactgacttg tttgtcttcc attccattgt tttgaaactc agtatgctgc ccctgtcttg
4681 ctgtcatgaa atcagcaaga gaggatgaca catcaaataa taactcggat tccagcccac
4741 attggattca tcagcatttg gaccaatagc ccacagctga gaatgtggaa tacctaagga
4801 tagcaccgct tttgttctcg caaaaacgta tctcctaatt tgaggctcag atgaaatgca
4861 tcaggtcctt tggggcatag atcagaagac tacaaaaatg aagctgctct gaaatctcct
4921 ttagccatca ccccaacccc ccaaaattag tttgtgttac ttatggaaga tagttttctc
4981 cttttacttc acttcaaaag ctttttactc aaagagtata tgttccctcc aggtcagctg
5041 cccccaaacc ccctccttac gctttgtcac acaaaaagtg tctctgcctt gagtcatcta
5101 ttcaagcact tacagctctg gccacaacag ggcattttac aggtgcgaat gacagtagca
5161 ttatgagtag tgtggaattc aggtagtaaa tatgaaacta gggtttgaaa ttgataatgc
5221 tttcacaaca tttgcagatg ttttagaagg aaaaaagttc cttcctaaaa taatttctct
5281 acaattggaa gattggaaga ttcagctagt taggagccca ccttttttcc taatctgtgt
5341 gtgccctgta acctgactgg ttaacagcag tcctttgtaa acagtgtttt aaactctcct
5401 agtcaatatc caccccatcc aatttatcaa ggaagaaatg gttcagaaaa tattttcagc
5461 ctacagttat gttcagtcac acacacatac aaaatgttcc ttttgctttt aaagtaattt
5521 ttgactccca gatcagtcag agcccctaca gcattgttaa gaaagtattt gatttttgtc
5581 tcaatgaaaa taaaactata ttcatttcca ctctattatg ctctcaaata cccctaagca
5641 tctatactag cctggtatgg gtatgaaaga tacaaagata aataaaacat agtccctgat
5701 tctaagaaat tcacaattta gcaaaggaaa tggactcata gatgctaacc ttaaaacaac
5761 gtgacaaatg ccagacagga cccatcagcc aggcactgtg agagcacaga gcagggaggt
5821 tgggtcctgc ctgaggagac ctggaaggga ggcctcacag gaggatgacc aggtctcagt
5881 cagcggggag gtggaaagtg caggtgcatc aggggcaccc tgaccgagga aacagctgcc
5941 agaggcctcc actgctaaag tccacataag gctgaggtca gtcaccctaa acaacctgct
6001 ccctctaagc caggggatga gcttggagca tcccacaagt tccctaaaag ttgcagcccc
6061 cagggggatt ttgagctatc atctctgcac atgcttagtg agaagactac acaacatttc
6121 taagaatctg agattttata ttgtcagtta accactttca ttattcattc acctcaggac
6181 atgcagaaat atttcagtca gaactgggaa acagaaggac ctacattctg ctgtcactta
6241 tgtgtcaaga agcagatgat cgatgaggca ggtcagttgt aagtgagtca cattgtagca
6301 ttaaattcta gtatttttgt agtttgaaac agtaacttaa taaaagagca aaagctattc
6361 tagctttctt cttcatattt taattttcca ccataaagtt tagttgctaa attctattaa
6421 ttttaagatt gtgcttccca aaatagttct cacttcatct gtccagggag gcacagttct
6481 gtctggtaga agccgcaaag cccttagcct cttcacggat ctggcgactg tgatgggcag
6541 gtcaggagag gagctgccca aagtcccatg attttcacct aacagccctg atcagtcagt
6601 actcaaagct tggactccat ccctgaaggt cttcctgatt gatagcctgg ccttaatacc
6661 ctacagaaag cctgtccatt ggctgtttct tcctcagtca gttcctggaa gaccttaccc
6721 catgacccca gcttcagatg tggtctttgg aaacagaggt cgaaggaaag taaggagctg
6781 agagctcaca ttcataggtg ccgccagcct tcgtgcatct tcttgcatca tctctaagga
6841 gctcctctaa ttacaccatg cccgtcaccc catgagggat cagagaaggg atgagtcttc
6901 taaactctat attcgctgtg agtccaggtt gtaaggggga gcactgtgga tgcatcctat
6961 tgcactccag ctgatgacac caaagcttag gtgtttgctg aaagttcttg atgttgtgac
7021 ttaccacccc tgcctcacaa ctgcagacat aaggggacta tggattgctt agcaggaaag
7081 gcactggttc tcaagggcgg ctgcccttgg gaatcttctg gtcccaacca gaaagactgt
7141 ggcttgattt tctcaggtgc agcccagccg tagggccttt tcagagcacc ccctggttat
7201 tgcaacattc atcaaagttt ctagaacctc tggcctaaag gaagggcctg gtgggatcta
7261 cttggcactc gctggggggc caccccccag tgccactctc actaggcctc tgattgcact
7321 tgtgtaggat gaagctggtg ggtgatggga actcagcacc tcccctcagg cagaaaagaa
7381 tcatctgtgg agcttcaaaa gaaggggcct ggagtctctg cagaccaatt caacccaaat
7441 ctcgggggct ctttcatgat tctaatgggc aaccagggtt gaaaccctta tttctagggt
7501 cttcagttgt acaagactgt gggtctgtac cagagccccc gtcagagtag aataaaaggc
7561 tgggtagggt agagattccc atgtgcagtg gagagaacaa tctgcagtca ctgataagcc
7621 tgagacttgg ctcatttcaa aagcgttcaa ttcatcctca ccagcagttc agctggaaag
7681 gggcaaatac ccccacctga gctttgaaaa cgccctggga ccctctgcat tctctaagta
7741 agttatagaa accagtctct tccctccttt gtgagtgagc tgctattcca cgtaggcaac
7801 acctgttgaa attgccctca atgtctactc tgcatttctt tcttgtgata agcacacact
7861 tttattgcaa cataatgatc tgctcacatt tccttgcctg ggggctgtaa aaccttacag
7921 aacagaaatc cttgcctctt tcaccagcca cacctgccat accaggggta cagctttgta
7981 ctattgaaga cacagacagg atttttaaat gtaaatctat ttttgtaact ttgttgcggg
8041 atatagttct ctttatgtag cactgaactt tgtacaatat atttttagaa actcattttt
8101 ctactaaaac aaacacagtt tactttagag agactgcaat agaatcaaaa tttgaaactg
8161 aaatctttgt ttaaaagggt taagttgagg caagaggaaa gccctttctc tctcttataa
8221 aaaggcacaa cctcattggg gagctaagct aggtcattgt catggtgaag aagagaagca
8281 tcgtttttat atttaggaaa ttttaaaaga tgatggaaag cacatttagc ttggtctgag
8341 gcaggttctg ttggggcagt gttaatggaa agggctcact gttgttacta ctagaaaaat
8401 ccagttgcat gccatactct catcatctgc cagtgtaacc ctgtacatgt aagaaaagca
8461 ataacatagc actttgttgg tttatatata taatgtgact tcaatgcaaa ttttattttt
8521 atatttacaa ttgatatgca tttaccagta taaactagac atgtctggag agcctaataa
8581 tgttcagcac actttggtta gttcaccaac agtcttacca agcctgggcc cagccaccct
8641 agagaagtta ttcagccctg gctgcagtga catcacctga ggagctttta aaagcttgaa
8701 gcccagctac acctcagacc gattaaacgc aaatctctgg ggctgaaacc caagcattcg
8761 tagtttttaa agctcctgag gtcattccaa tgtgcggcca aagttgagaa ctactggcct
8821 agggattagc cacaaggaca tggacttgga ggcaaattct gcaggtgtat gtgattctca
8881 ggcctagaga gctaagacac aaagacctcc acatctgtcg ctgagagtca agaacctgaa
8941 cagagtttcc atgaaggttc tccaagcact agaagggaga gtgtctaaac aatggttgaa
9001 aagcaaagga aatataaaac agacacctct ttccatttcc taaggtttct ctctttatta
9061 agggtggact agtaataaaa tataatattc ttgctgctta tgcagctgac attgttgccc
9121 tccctaaagc aaccaagtag cctttatttc ccacagtgaa agaaaacgct ggcctatcag
9181 ttacattaca aaaggcagat ttcaagagga ttgagtaagt agttggatgg ctttcataaa
9241 aacaagaatt caagaagagg attcatgctt taagaaacat ttgttataca ttcctcacaa
9301 attatacctg ggataaaaac tatgtagcag gcagtgtgtt ttccttccat gtctctctgc
9361 actacctgca gtgtgtcctc tgaggctgca agtctgtcct atctgaattc ccagcagaag
9421 cactaagaag ctccacccta tcacctagca gataaaacta tggggaaaac ttaaatctgt
9481 gcatacattt ctggatgcat ttacttatct ttaaaaaaaa aggaatccta tgacctgatt
9541 tggccacaaa aataatcttg ctgtacaata caatctcttg gaaattaaga gatcctatgg
9601 atttgatgac tggtattaga ggtgacaatg taaccgatta acaacagaca gcaataactt
9661 cgttttagaa acattcaagc aatagcttta tagcttcaac atatggtacg ttttaacctt
9721 gaaagttttg caatgatgaa agcagtattt gtacaaatga aaagcagaat tctcttttat
9781 atggtttata ctgttgatca gaaatgttga ttgtgcattg agtattaaaa aattagatgt
9841 atattattca ttgttcttta ctcctgagta ccttataata ataataatgt attctttgtt
9901 aacaa
SEQ ID NO: 130
MRPSGTAGAA LLALLAALCP ASRALEEKKV CQGTSNKLTQ LGTFEDHELS LQRMENNCEV
VLGNLEITYV QRNYDLSFLK TIQEVAGYVL IALNTVERIP LENLQIIRGN MYYENSYALA
VLSNYDANKT GLKELPMRNL QEILHGAVRF SNNPALCNVE SIQWRDIVSS DELSNMSMDF
QNHLGSCQKC DPSCPNGSCW GAGEENCQKL TKIICAQQCS GRCRGKSPSD CCHNQCAAGC
TGPRESDCLV CRKFRDEATC KDTCPPLMLY NPTTYQMDVN PEGKYSFGAT CVKKCPRNYV
VTDHGSCVRA CGADSYEMEE DGVRKCKKCE GPCRKVCNGI GIGEFKDSLS INATNIKHFK
NCTSISGDLH ILPVAFRGDS FTHTPPLDPQ ELDILKTVKE ITGFLLIQAW PENRTDLHAF
ENLEIIRGRT KQHGQFSLAV VSLNITSLGL RSLKEISDGD VIISGNKNLC YANTINWKKL
FGTSGQKTKI ISNRGENSCK ATGQVCHALC SPEGCWGPEP RDCVSCRNVS RGRECVDKCN
LLEGEPREFV ENSECIQCHP ECLPQAMNIT CTGRGPDNCI QCAHYIDGPH CVKTCPAGVM
GENNTLVWKY ADAGHVCHLC HPNCTYGCTG PGLEGCPTNG PKIPSIATGM VGALLLLLVV
ALGIGLEMRR RHIVRKRTLR RLLQERELVE PLTPSGEAPN QALLRILKET EFKKIKVLGS
GAFGTVYKGL WIPEGEKVKI PVAIKELREA TSPKANKEIL DEAYVMASVD NPHVCRLLGI
CLTSTVQLIT QLMPEGCLLD YVREHKDNIG SQYLLNWCVQ IAKGMNYLED RRLVHRDLAA
RNVLVKTPQH VKITDFGLAK LLGAEEKEYH AEGGKVPIKW MALESILHRI YTHQSDVWSY
GVTVWELMTF GSKPYDGIPA SEISSILEKG ERLPQPPICT IDVYMIMVKC WMIDADSRPK
FRELIIEFSK MARDPQRYLV IQGDERMHLP SPIDSNFYRA LMDEEDMDDV VDADEYLIPQ
QGFFSSPSTS RTPLLSSLSA TSNNSTVACI DRNGLQSCPI KEDSFLQRYS SDPTGALTED
SIDDTFLPVP EYINQSVPKR PAGSVQNPVY HNQPLNPAPS RDPHYQDPHS TAVGNPEYLN
TVQPTCVNST FDSPAHWAQK GSHQISLDNP DYQQDFFPKE AKPNGIFKGS TAENAEYLRV
APQSSEFIGA
```

### CRISPR/Endonucleases

CRISPR/endonuclease (e.g., CRISPR/Cas9) systems are known in the art and are described, for example, in U.S. Pat. No. 9,925,248, which is incorporated by reference herein in its entirety. CRISPR-directed gene editing can identify and execute DNA cleavage at specific sites within the chromosome at a surprisingly high efficiency and precision. The natural activity of CRISPR/Cas9 is to disable a viral genome infecting a bacterial cell. Subsequent genetic reengineering of CRISPR/Cas function in human cells presents the possibility of disabling human genes at a significant frequency.

In bacteria, the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA) containing a DNA binding region (spacer) which is complementary to the target gene. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called a spacer) and a trans-activated small RNA (tracrRNA) that serves as a guide for ribonuclease III-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM).

The compositions described herein can include a nucleic acid encoding a CRISPR-associated endonuclease. The CRISPR-associated endonuclease can be, e.g., a class 1 CRISPR-associated endonuclease or a class 2 CRISPR-associated endonuclease. Class 1 CRISPR-associated endonucleases include type I, type III, and type IV CRISPR-Cas systems, which have effector molecules that comprise multiple subunits. For class 1 CRISPR-associated endonucleases, effector molecules can include, in some embodiments, Cas7 and Cas5, along with, in some embodiments, SS (Cas11) and Cas8a1; Cas8b1; Cas8c; Cas8u2 and Cas6; Cas3″ and Cas10d; Cas SS (Cas11), Cas8c, and Cas6; Cas8f and Cas6f; Cas6f; Cas8-like (Csf1); SS (Cas11) and Cas8-like (Csf1); or SS (Cas11) and Cas10. Class 1 CRISPR-associated endonucleases also be associated with, in some embodiments, target cleavage molecules, which can be Cas3 (type I) or Cas10 (type III) and spacer acquisition molecules such as, e.g., Cas1, Cas2, and/or Cas4. See, e.g., Koonin et al., Curr. Opin. Microbiol. 37:67-78 (2017); Strich & Chertow, J. Clin. Microbiol. 57:1307-18 (2019).

Class 2 CRISPR-associated endonucleases include type I, type V, and type VI CRISPR-Cas systems, which have a single effector molecule. For class 2 CRISPR-associated endonucleases, effector molecules can include, in some embodiments, Cas9, Cas12a (cpf1), Cas12b1 (c2c1), Cas12b2, Cas12c (c2c3), Cas12d (CasY), Cas12e (CasX), Cas12f1 (Cas14a), Cas12f2 (Cas14b), Cas12f3 (Cas14c), Cas12g, Cas12h, Cas12i, Cas12k (c2c5), Cas13a (c2c2), Cas13b1 (c2c6), Cas13b2 (c2c6), Cas13c (c2c7), Cas13d, c2c4, c2c8, c2c9, and/or c2c10. See, e.g., Koonin et al., Curr. Opin. Microbiol. 37:67-78 (2017); Strich & Chertow, J. Clin. Microbiol. 57:1307-18 (2019); Makarova et al., Nat. Rev. Microbiol. 18:67-83 (2020).

In some embodiments, the CRISPR-associated endonuclease can be a Cas9 nuclease. The Cas9 nuclease can be encoded by a nucleotide sequence identical to the wild type Streptococcus pyogenes nucleotide sequence. In some embodiments, the nucleic acid sequence that encodes the Cas9 nuclease comprises or consists of the nucleic acid sequence of SEQ ID NO: 115, or a nucleic acid sequence having at least 80%, 85%, 90%, 95%, 96%, 97%, 98% or 99% sequence identity to SEQ ID NO: 115. In some embodiments, the Cas9 nuclease comprises or consists of the amino acid sequence of SEQ ID NO: 114, or an amino acid sequence having at least 80%, 85%, 90%, 95%, 96%, 97%, 98% or 99% sequence identity to SEQ ID NO: 114. In some embodiments, the CRISPR-associated endonuclease is the small Cas9 ortholog from Staphylococcus aureus (SaCas9), or a variant thereof. SaCas9 endonucleases and variants thereof are described, for example, in Tan et al., 2019, PNAS 116 (42) 20969-20976, which is incorporated by reference herein in its entirety.

In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Such species include: Acidovorax avenue, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., and Verminephrobacter eiseniae.

Alternatively, the wild type Streptococcus pyogenes Cas9 sequence can be modified. The nucleic acid sequence can be codon optimized for efficient expression in mammalian cells, e.g., human cells. A Cas9 nuclease sequence codon optimized for expression in human cells sequence can be for example, the Cas9 nuclease sequence encoded by any of the expression vectors listed in Genbank accession numbers KM099231.1 GI: 669193757; KM099232.1 GI: 669193761; or KM099233.1 GI: 669193765. Alternatively, the Cas9 nuclease sequence can be, for example, the sequence contained within a commercially available vector such as pX458, pX330 or pX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI: 669193757; KM099232.1 GI: 669193761; or KM099233.1 GI: 669193765 or Cas9 amino acid sequence of pX458, pX330 or pX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a wild type Cas9 polypeptide.

In some embodiments, the CRISPR-associated endonuclease can be a Cas12a nuclease. The Cas12a nuclease can have a nucleotide sequence identical to a wild type Prevotella or Francisella sequence. Alternatively, a wild type Prevotella or Francisella Cas12a sequence can be modified. The nucleic acid sequence can be codon optimized for efficient expression in mammalian cells, e.g., human cells. A Cas12a nuclease sequence codon optimized for expression in human cells sequence can be for example, the Cas9 nuclease sequence encoded by any of the expression vectors listed in Genbank accession numbers MF193599.1 GI: 1214941796, KY985374.1 GI: 1242863785, KY985375.1 GI: 1242863787, or KY985376.1 GI: 1242863789. Alternatively, the Cas12a nuclease sequence can be, for example, the sequence contained within a commercially available vector such as pAs-Cpf1 or pLb-Cpf1 from Addgene (Cambridge, Mass.). In some embodiments, the Cas12a endonuclease can have an amino acid sequence that is a variant or a fragment of any of the Cas12a endonuclease sequences of Genbank accession numbers MF193599.1 GI: 1214941796, KY985374.1 GI: 1242863785, KY985375.1 GI: 1242863787, or KY985376.1 GI: 1242863789 or Cas12a amino acid sequence of pAs-Cpf1 or pLb-Cpf1 (Addgene, Cambridge, Mass.). The Cas12a nucleotide sequence can be modified to encode biologically active variants of Cas12a, and these variants can have or can include, for example, an amino acid sequence that differs from a wild type Cas12a by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas12a polypeptide can have an amino acid sequence with at least or about 50% sequence identity (e.g., at least or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a wild type Cas12a polypeptide.

In some embodiments, the CRISPR-associated endonuclease is an IscB protein, an IsrB protein or a TnpB protein. These proteins are described, for example, in H. Altac-Tran et al., 2021, Science 10.1126/science.abj6856, which is incorporated by reference herein in its entirety.

The adenoviruses described herein may also include a polynucleotide encoding a guide RNA (gRNA) comprising a DNA-binding domain that is complementary to a target domain from a gene (e.g., an NRF2 gene, for example, a target domain from exon 1, 2, 3, 4, or 5 of an NRF2 gene), and a Clustered Regularly Interspaced Short Palindromic Repeat (CRISPR)-associated endonuclease protein-binding domain. In some embodiments, the gRNA comprises a DNA-binding domain that is complementary to a target domain from a variant gene (e.g., a variant NRF2 gene, for example, a target domain from exon 1, 2, 3, 4, or 5 of a variant NRF2 gene) that is found only in cancer cells and not in a corresponding wild-type gene in normal (i.e., non-cancerous) cells. The guide RNA sequence can be a sense or anti-sense sequence. The guide RNA sequence may include a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. The specific sequence of the guide RNA may vary, but, regardless of the sequence, useful guide RNA sequences will be those that minimize off-target effects while achieving high efficiency. In some embodiments, the guide RNA sequence achieves complete ablation of the target gene (e.g., the NRF2 gene). In some embodiments, the guide RNA sequence achieves complete ablation of a variant gene (e.g., an NRF2 gene) without affecting expression or activity of the corresponding wild-type gene (e.g., the wild-type NRF2 gene).

In some embodiments, at least one of the gRNAs encoded by the adenovirus hybridizes to a target sequence comprising a PAM site in the variant gene that results from a mutation to the variant gene creating the PAM site that does not exist in the wild-type gene. In some embodiments, at least one of the gRNAs hybridizes to a target sequence comprising a PAM site in the variant gene that is operably linked to a mutated portion of the wild-type gene. The term “mutation” means a variation from a known reference sequence and includes mutations such as, for example, single nucleotide variants (SNVs), copy number variants or variations (CNVs)/aberrations, insertions or deletions (indels), gene fusions, transversions, translocations, frame shifts, duplications, repeat expansions, and epigenetic variants. A mutation can be a germline or somatic mutation. Mutations can be caused by, e.g., one or more nucleotide additions, substitutions, or deletions. In some embodiments, one or more mutations in a nucleotide sequence produce a new PAM site as compared to a wild-type nucleotide sequence that does not contain such PAM site. In some embodiments, one or more mutations in a nucleotide sequence shift a PAM site to a different location in the nucleotide sequence (e.g., from an intron to an exon, from an exon to an intron, from a position upstream in a gene to a position downstream, or from a position downstream in a gene to a position upstream) as compared to the PAM site's location in a wild-type nucleotide sequence.

In some embodiments, the mutation is a fusion between two genes where the resulting fusion creates a new PAM site. In some embodiments, the PAM site is newly created as between one of the two genes that forms the fusion gene (i.e., the new fusion gene contains a PAM site that already existed in one of the two contributors to the fusion gene), and in some embodiments, the PAM site is new created as between both genes that form the fusion gene (i.e., the new fusion gene contains a new PAM site that does not exist in either contributor to the fusion gene).

In some embodiments, at least one of the gRNAs hybridizes to a target sequence comprising a PAM site in an intron of the variant gene downstream or upstream from the PAM site discussed above. In some embodiments, the PAM site in an intron of the variant gene can be present in the same location in the wild-type gene. In some embodiments, the PAM site in an intron of the variant gene is not present in the related wild-type gene.

In some embodiments, the DNA-binding domain of the gRNA varies in length from about 20 to about 55 nucleotides, for example, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, or about 55 nucleotides. In some embodiments, the Cas protein-binding domain is from about 30 to about 55 nucleotides in length, for example, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, or about 55 nucleotides.

### Polynucleotides Encoding the Guide RNA and CRISPR Endonuclease

In some embodiments, the adenoviruses described herein comprise one or more nucleic acid (i.e., DNA) molecules encoding the guide RNA and one or more nucleic acid (i.e., DNA) molecules encoding the CRISPR endonuclease. In some embodiments, the CRISPR endonuclease and the guide RNA are encoded by separate adenoviruses. In some embodiments, the nucleic acid molecule encoding the guide RNA comprises a nucleic acid sequence encoding a DNA binding domain, a nucleic acid sequence encoding a Cas protein binding domain, and a transcription terminator domain. In some embodiments, the nucleic acid molecule encoding the guide RNA comprises a nucleic acid sequence encoding a DNA binding domain, and a nucleic acid sequence encoding a Cas protein binding domain. In some embodiments the nucleic acid molecule encoding the DNA binding domain comprises or consists of the nucleic acid sequence of SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 59, SEQ ID NO: 60, SEQ ID NO: 61, SEQ ID NO: 62, SEQ ID NO: 63, or SEQ ID NO: 84, or a nucleic acid sequence having at least 80%, 85%, 90%, 95%, 96%, 97%, 98% or 99% sequence identity to the nucleic acid sequence of SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 59, SEQ ID NO: 60, SEQ ID NO: 61, SEQ ID NO: 62, SEQ ID NO: 63, or SEQ ID NO: 84, or a biologically active fragment thereof. In a particular embodiment, the nucleic acid molecule encoding the DNA binding domain comprises or consists of the nucleic acid sequence of SEQ ID NO: 61, or a nucleic acid sequence having at least 80%, 85%, 90%, 95%, 96%, 97%, 98% or 99% sequence identity to the nucleic acid sequence of SEQ ID NO: 61, or a biologically active fragment thereof. In a further particular embodiment, the nucleic acid molecule encoding the DNA binding domain comprises or consists of the nucleic acid sequence of SEQ ID NO: 61.

TABLE 2
DNA binding domains of cancer-derived gRNAs
from variant NRF2s (substitution in bold).
The guide RNA recognition element encodes
the DNA binding domain of the
corresponding guide RNA.

| | | |
|---|---|---|
| SEQ ID NO: 17 | Guide RNA3 (gRNA3) | 3′-AAACTAACTGTATGAAACCTCCC-5′ |
| | recognition element | |
| SEQ ID NO: 18 | Guide RNA3 (gRNA3) | 5′-UUUGAUUGACAUACUUUGGAGGG-3′ |
| SEQ ID NO: 19 | Guide RNA4 (gRNA4) | 3′-GAATGAGGTTCTAGATATAGACC-5′ |
| | recognition element | |
| SEQ ID NO: 20 | Guide RNA4 (gRNA4) | 5′-CUUACUCCAAGAUCUAUAUCUGG-3′ |
| SEQ ID NO: 21 | Guide RNA5 (gRNA5) | 3′-TATGAAACCTCCGTTCTATATCC-5′ |
| | recognition element | |
| SEQ ID NO: 22 | Guide RNA5 (gRNA5) | 5′-AUACUUUGGAGGCAAGAUAUAGG-3′ |
| SEQ ID NO: 23 | Guide RNA6 (gRNA6) | 3′-TCCGTTCTATATCTAGAACCTCC-5′ |
| | recognition element | |
| SEQ ID NO: 24 | Guide RNA6 (gRNA6) | 5′-AGGCAAGAUAUAGAUCUUGGAGG-3′ |
| SEQ ID NO: 25 | Guide RNA7 (gRNA7) | 3′-CTATATCTAGAACCTCATTCACC-5′ |
| | recognition element | |
| SEQ ID NO: 26 | Guide RNA7 (gRNA7) | 5′-GAUAUAGAUCUUGGAGUAAGUGG-3′ |
| SEQ ID NO: 27 | Guide RNA8 (gRNA8) | 3′-ACTGACTTCAGTTTATGAAGACC-5′ |
| | recognition sequence | |
| SEQ ID NO: 28 | Guide RNA8 (gRNA8) | 5′-UGACUGAAGUCAAAUACUUCUGG-3′ |
| SEQ ID NO: 29 | Guide RNA9 (gRNA9) | 3′-AAGTAGATCAACATTGACTCGCC-5′ |
| | recognition sequence | |
| SEQ ID NO: 30 | Guide RNA9 (gRNA9) | 5′-UUCAUCUAGUUGUAACUGAGCGG-3′ |
| SEQ ID NO: 31 | Guide RNA10 (gRNA10) | 3′-TAAGTGGACAGAGAAGTAGATCC-5′ |
| | recognition sequence | |
| SEQ ID NO: 32 | Guide RNA10 (gRNA10) | 5′-AUUCACCUGUCUCUUCAUCUAGG-3′ |
| SEQ ID NO: 33 | Guide RNA11 (gRNA11) | 3′-GAGTCAATGTTGATCTACTTCCC-5′ |
| | recognition sequence | |
| SEQ ID NO: 34 | Guide RNA11 (gRNA11) | 5′-CUCAGUUACAACUAGAUGAAGGG-3′ |
| SEQ ID NO: 35 | Guide RNA12 (gRNA12) | 3′-ACTTAACCCTCTTTAAGTGGACC-5′ |
| | recognition sequence | |
| SEQ ID NO: 36 | Guide RNA12 (gRNA12) | 5′-UGAAUUGGGAGAAAUUCACCUGG-3′ |
| SEQ ID NO: 37 | Guide RNA13 (gRNA13) | 3′-GTTGATCTACTTCTCTGTCCACC-5′ |
| | recognition sequence | |
| SEQ ID NO: 38 | Guide RNA13 (gRNA13) | 5′-CAACUAGAUGAAGAGACAGGUGG-3′ |
| SEQ ID NO: 39 | Guide RNA14 (gRNA14) | 3′-TATTATCGAGGAGGGTTTGAACC-5′ |
| | recognition sequence | |
| SEQ ID NO: 40 | Guide RNA14 (gRNA14) | 5′-AUAAUAGCUCCUCCCAAACUUGG-3′ |
| SEQ ID NO: 53 | Guide RNA15 (gRNA15) | 3′-AAAAAGCGAGTAATGTTGATCC-5′ |
| | recognition sequence | |
| SEQ ID NO: 54 | Guide RNA15 (gRNA15) | 5′-UUUUUCGCUCAGUUACAACUAGG-3′ |
| SEQ ID NO: 55 | Guide RNA16 (gRNA16) | 3′-GTTGATCTACTTCTCTGTCCACC-5′ |
| | recognition sequence | |
| SEQ ID NO: 56 | Guide RNA16 (gRNA16) | 5′-CAACUAGAUGAAGAGACAGGUGG-3′ |

PAM sequence underlined.

In some embodiments, the nucleic acid molecule encoding the Cas (e.g. Cas9) protein binding domain comprises of consists of the nucleic acid sequence of SEQ ID NO: 113 or a nucleic acid sequence having at least 80%, 85%, 90%, 95%, 96%, 97%, 98% or 99% sequence identity to the nucleic acid sequence of SEQ ID NO: 113, or a biologically active fragment thereof. In some embodiments, the DNA molecule encoding the guide RNA comprises the nucleic acid sequence of SEQ ID NO: 61 (encoding the DNA binding domain), and the nucleic acid sequence of SEQ ID NO: 113 (encoding a Cas9 binding domain). For example, in some embodiments, the DNA molecule encoding the gRNA comprises or consists of the nucleic acid sequence of SEQ ID NO: 124, or a nucleic acid sequence having at least 80%, 85%, 90%, 95%, 96%, 97%, 98% or 99% sequence identity to the nucleic acid sequence of SEQ ID NO: 124, or a biologically active fragment thereof. SEQ ID NO: 124 consists of the nucleic acid sequence of SEQ ID NO: 61 (encoding the DNA binding domain), and the nucleic acid sequence of SEQ ID NO: 113 (encoding a Cas9 binding domain).

The nucleic acid molecule encoding the guide RNA and/or the CRISPR endonuclease may be an isolated nucleic acid. An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids also can be obtained by mutagenesis of, e.g., a naturally occurring portion of a Cas9-encoding DNA (in accordance with, for example, the formula above).

Recombinant adenoviruses are also provided herein and can be used to transfect cells in order to express the CRISPR endonuclease and/or a guide RNA complementary to a target gene (e.g., an NRF2 gene, in some embodiments, a variant NRF2 gene found only in cancer cells). A recombinant nucleic acid construct may comprise a nucleic acid encoding a CRISPR endonuclease and/or a guide RNA complementary to a target gene (e.g., an NRF2 gene, in some embodiments, a variant NRF2 gene found only in cancer cells), operably linked to a promoter suitable for expressing the CRISPR endonuclease and/or a guide RNA complementary to the target gene (e.g., an NRF2 gene, in some embodiments, the variant NRF2 gene) in the cell. In some embodiments the nucleic acid encoding a CRISPR endonuclease is operably linked to the same promoter as the nucleic acid encoding the guide RNA. In other embodiments, the nucleic acid encoding a CRISPR endonuclease and the nucleic acid encoding the guide RNA are operably linked to different promoters. In some embodiments, the nucleic acid encoding a CRISPR endonuclease and/or the nucleic acid encoding a guide RNA are operably linked to a lung specific promoter. Suitable lung specific promoters include, but are not limited to, Clara cell 10-kDa protein (CC₁₀) (aka Scgb1a1) promoter (Stripp et al., J. Biol. Chem. 267:14703-12 (1992)), SFTPC promoter (Wert et al., Dev. Biol. 156:426-43 (1993)), FOXJ1 promoter (Ostrowski et al., Mol. Ther. 8:637-45 (2003)), aquaporin (Aqp5) promoter (Funaki et al., Am. J. Physiol. 275: C1151-57 (1998)), Keratin 5 (Krt5) promoter (Rock et al., Dis. Model Mech. 3:545-56 (2010)), Keratin 14 (Krt14) promoter (Rock et al., Dis. Model Mech. 3:545-56 (2010)), cytokeratin 18 (K18) promoter (Chow et al., Proc. Natl. Acad. Sci. USA 94:14695-14700 (1997)), surfactant protein B (SP-B) promoter (Strayer et al., Am. J. Physiol. Lung Cell Mol. Physiol. 282: L394-404 (2002)), TTF1 gene under the control of human telomerase reverse transcriptase promoter and human surfactant protein A1 promoter (Fukazawa et al., Cancer Res. 64:363-69 (2004)), surfactant protein C (SP-C) promoter (Zhuo et al., Transgenic Res. 15:543-55 (2006)), insulinoma-associated antigen-1 (INSM1) promoter (Li et al., Biochem. Biophys. Res. Commun. 236:776-81 (1997)), and surfactant protein A (SP-A) promoter (Bruno et al., J. Biol. Chem. 270:6531-36 (1995)).

The adenoviruses described herein can also include one or more regulatory regions. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region (e.g. a promoter) and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2.000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

In certain aspects, the disclosure relates to an adenovirus comprising a polynucleotide, wherein the polynucleotide comprises: (a) a first DNA molecule encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in a gene; and (b) a first promoter that is operably linked to the DNA molecule. In some embodiments, the gene targeted by the gRNA is selected from the group consisting of NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7, BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNXIT1, DNMT3A, SMCIA, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST, TSC2, EEF1A1, WHSC1, APOB, NUP133, AXIN1, PHF6, TET2, WT1, FLT3, FLT4, SMC3, CEBPA, RAD21, RAD50, RAD51, PTPDC1, ASXL1, EZH2, NPM1, SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA, LATS1, POLRMT, CDK4, COL5A1, PPP6C, MECOM, DACH1, MAP2K1, MAP2K2, RQCD1, DDX3X, NUP93, PPMID, CHD2, CHD3, CCND1, CCND2, CCND3, ACVR1, KMT2A, KMT2B, KMT2C, KMT2D, SIN3A, SCAF4, DICER1, FOXA2, CTNND1, MYC, MYCL, MYCN, SOX17, ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF2I, RIT1, MGA, ABL1, BRAF, CHEK1, FANCC, JAK2, MITF, PDCDILG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSFIR, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKBIA, PRDM1, DOT1L, FGFR1, LMO1, NKX2-1, PREX2, HSD3B1, LRP1B, TSHR, ATRX, CDC73, HSP90AA1, PRKCI, AURKA, PRKDC, VEGFA, AURKB, CDK12, FH, MAGI2, PRSS8, SMO, FLCN, IGFIR, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B, PARK2, RANBP2, SRC, IRS2, PAX5, RARA, BLM, FANCA, JAK1, FCRL4, LIG4, MAR, PWWP3A, MUC16, MUC17, FCGBP, FAT17, MMSET, IRTA2, TTN, DST, and STAT3,

In some embodiments, the gene targeted by the gRNA is an NRF2 gene. In some embodiments, the NRF2 gene is a wildtype NRF2 gene, e.g. an NRF2 gene that naturally occurs in normal cells (e.g. non-cancerous cells). In some embodiments, the NRF2 gene is a variant NRF2 gene. In some embodiments, the variant NRF2 gene is a variant that is found in normal cells (e.g. non-cancerous cells). In some embodiments, the variant NRF2 gene is a variant that is found in cancer cells. In a particular embodiment the variant NRF2 gene is a variant found in lung cancer cells, e.g. non-small cell lung cancer (NSCLC) cells or lung squamous cell carcinoma. In some embodiments, the variant NRF2 gene encodes an NRF2 polypeptide comprising one or more amino acid substitutions selected from the group consisting of Q26E, Q26P. D29G, V32G, R34G, R34P, F71S, Q75H, E79G, T80P, E82W, and E185D relative to the amino acid sequence of SEQ ID NO: 8. In a particular embodiment, the NRF2 polypeptide comprises an R34G substitution relative to the amino acid sequence of SEQ ID NO: 8. In some embodiments, the gRNA is complementary to a target sequence in exon 2 of the variant NRF2 gene. In some embodiments, the DNA-binding domain comprises the nucleic acid sequence of SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, or SEQ ID NO: 126, or a biologically active fragment thereof. In some embodiments, the DNA sequence encoding the DNA binding domain of the gRNA comprises the nucleic acid sequence of SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 59, SEQ ID NO: 60, SEQ ID NO: 61, SEQ ID NO: 62, SEQ ID NO: 63, SEQ ID NO: 84, or a biologically active fragment thereof. In some embodiments, the DNA sequence encoding the (CRISPR)-associated endonuclease protein-binding domain of the gRNA comprises or consists of the nucleic acid sequence of SEQ ID NO: 113, or a nucleic acid sequence having at least 80%, 85%, 90%, 95%, 96%, 97%, 98% or 99% sequence identity to SEQ ID NO: 113, or a biologically active fragment thereof.

In some embodiments, the DNA binding domain of the gRNA comprises or consists of the RNA sequence of SEQ ID NO: 126, or an RNA sequence having at least 80%, 85%, 90%, 95%, 96%, 97%, 98% or 99% sequence identity to SEQ ID NO: 126, or a biologically active fragment thereof. In some embodiments, the (CRISPR)-associated endonuclease protein-binding domain of the gRNA comprises or consists of the RNA sequence of SEQ ID NO: 127, or an RNA sequence having at least 80%, 85%, 90%, 95%, 96%, 97%, 98% or 99% sequence identity to SEQ ID NO: 127, or a biologically active fragment thereof. In some embodiments, the gRNA comprises or consists of the RNA sequence of SEQ ID NO: 128, or an RNA sequence having at least 80%, 85%, 90%, 95%, 96%, 97%, 98% or 99% sequence identity to SEQ ID NO: 128, or a biologically active fragment thereof. The DNA binding domain of SEQ ID NO: 126 and SEQ ID NO: 128 is encoded by the nucleic acid sequence of SEQ ID NO: 61.

The polynucleotide encoding the gRNA may comprise two or more repeats (e.g. tandem repeats) of a DNA sequence encoding the gRNA with each DNA sequence being operably linked to a promoter. The polynucleotide may contain 2, 3, 4, 5 or more repeats (e.g. tandem repeats) of the DNA sequence encoding the gRNA, with each DNA sequence being operably linked to a promoter. In some embodiments, each promoter operably linked to the DNA sequence encoding the gRNA is the same promoter. In some embodiments, the promoters operably linked to the DNA sequence encoding the gRNA may be different promoters.

For example, the polynucleotide comprising the first DNA sequence encoding the gRNA operably linked to a first promoter may further comprise (a) a second DNA sequence encoding the gRNA; and (b) a second promoter that is operably linked to the second DNA sequence. In some embodiments, the polynucleotide further comprises: (a) a third DNA sequence encoding the gRNA; and (b) a third promoter that is operably linked to the third DNA sequence.

In some embodiments, multiple sgRNAs may be expressed through the use of tRNA processing machinery. In this system, tRNA genes are fused to polynucleotide sequence encoding sgRNAs, such that multiple active sgRNAs can be produced from a single precursor transcript containing the tRNAs through tRNA processing. tRNA-based multiplex sgRNA expression systems are known in the art and are described, for example, in Shiraki et al., 2018, Sci. Rep. 8:13366.

The length of the DNA molecule encoding the one or more sgRNAs and comprising one or more promoters driving expression of the sgRNAs may be at least 1, 1.5, 2, 2.5, 3, 3.5, 4, 4.5, 5, 6, 7 or 8 kb. In some embodiments, the length of the DNA molecule encoding the one or more sgRNAs and comprising one or more promoters driving expression of the sgRNAs may be less than 1.5, 2, 2.5, 3, 3.5, 4, 4.5, 5, 6, 7 or 8 kb. Any of these values may be used to define a range for the length of the DNA molecule encoding the one or more sgRNAs and comprising one or more promoters driving expression of the sgRNAs. For example, in some embodiments, the DNA molecule encoding the one or more sgRNAs and comprising one or more promoters driving expression of the sgRNAs is 1-3 kb, 1-4 kb or 1-5 kb in length. In a particular embodiment, the DNA molecule encoding the one or more sgRNAs and comprising one or more promoters driving expression of the sgRNAs is at least 1 kb in length.

The length of the DNA molecule encoding the one or more sgRNAs and comprising one or more promoters driving expression of the sgRNAs, and further comprising a DNA molecule encoding a CRISPR-associated endonuclease may be at least 2, 2.5, 3, 3.5, 4, 4.5, 5, 6, 7 or 8 kb. In some embodiments, the length of the DNA molecule encoding the one or more sgRNAs and comprising one or more promoters driving expression of the sgRNAs, and further comprising a DNA molecule encoding a CRISPR-associated endonuclease may be less than 2, 2.5, 3, 3.5, 4, 4.5, 5, 6, 7 or 8 kb. Any of these values may be used to define a range for the length of the DNA molecule encoding the one or more sgRNAs and comprising one or more promoters driving expression of the sgRNAs, and further comprising a DNA molecule encoding a CRISPR-associated endonuclease. For example, in some embodiments, the DNA molecule encoding the one or more sgRNAs and comprising one or more promoters driving expression of the sgRNAs and further comprising a DNA molecule encoding a CRISPR-associated endonuclease is 2-3 kb, 2-4 kb. 2-5 kb, 2-6 kb or 2-7 kb in length. In a particular embodiment, the DNA molecule encoding the one or more sgRNAs and comprising one or more promoters driving expression of the sgRNAs, and further comprising a DNA molecule encoding a CRISPR-associated endonuclease is at least 5 kb in length.

In some embodiments, an adenovirus may comprise one or more RNA polymerase III (pol III) promoters (e.g. 1, 2, 3, 4, 5, or more pol III promoters), one or more RNA polymerase II (pol II) promoters (e.g. 1, 2, 3, 4, 5, or more pol II promoters), one or more tissue-specific promoters (e.g. 1, 2, 3, 4, 5, or more tissue-specific promoters), or combinations thereof.

In some embodiments, the promoter operably linked to the DNA molecule encoding the gRNA is a pol III promoter. A number of Pol III promoters are known in the art, including the U6 small nuclear (sn) RNA promoter, 7SK promoter, and the H1 promoter. Sec, e.g., Ro et al., BioTechniques, 38 (4): 625-627 (2005). In some embodiments, the U6 promoter comprises or consists of the nucleic acid sequence of SEQ ID NO: 118, or a biologically active fragment thereof. In some embodiments, the 7SK promoter comprises or consists of the nucleic acid sequence of SEQ ID NO: 123, or a biologically active fragment thereof. In some embodiments, the H1 promoter comprises or consists of the nucleic acid sequence of SEQ ID NO: 122, or a biologically active fragment thereof.

In some embodiments, the promoter operably linked to the DNA molecule encoding the CRISPR-associated endonuclease is a pol II promoter. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the minimal CMV promoter (without the CMV enhancer), the SV40 promoter, the dihydrofolate reductase promoter, the β-actin promoter, the phosphoglycerol kinase (PGK) promoter, the elongation factor 1α (EF1α) promoter, the elongation factor 1α short (EFS) promoter, the CAG promoter (see, e.g., Alexopoulou et al., BMC Cell Biology 9:2, 2008; Miyazaki et al., Gene 79 (2): 269-77 (1989); Niwa et al., Gene 108 (2): 193-9 (1991); the CAGGS promoter, the PGK promoter, the UbiC promoter. In some embodiments, the promoter operably linked to the DNA molecule encoding the CRISPR-associated endonuclease is an H1 promoter, e.g., an H1 promoter comprising or consisting of the nucleic acid sequence of SEQ ID NO: 122, or a biologically active fragment thereof.

In some embodiments, the promoter operably linked to the DNA sequence encoding the gRNA is a tissue-specific promoter. Examples of tissue-specific promoters include, but are not limited to, B29, Desmin, Endoglin, FLT-1, GFPA and SYN1.

The CMV and PGK promoters can be amplified from pSicoR and pSicoR PGK respectively (Ventura et al., Proc Natl Acad Sci USA 101:10380-10385 (2004)), the UbiC promoter can be amplified from pDSL_hpUGIH (ATCC), the CAGGS promoter can be amplified from pCAGGS (BCCM), and the EF1A promoter can be amplified from the pEF6 vector (Invitrogen). The Pol II core promoter is described in Butler and Kadonaga, Genes & Dev. 16:2583-2592 (2002). In some embodiments, the CMV promoter comprises or consists of the nucleic acid sequence of SEQ ID NO: 119.

In particular embodiments of the polynucleotides encoding gRNAs as described herein, the first promoter, the second promoter, and the third promoter are selected from the group consisting of U6, H1 and 7SK. In some embodiments, the first promoter is U6, the second promoter is H1, and the third promoter is 7SK. In some embodiments, the U6 promoter comprises or consists of the nucleic acid sequence of SEQ ID NO: 118, or a biologically active fragment thereof. In some embodiments, the 7SK promoter comprises or consists of the nucleic acid sequence of SEQ ID NO: 123, or a biologically active fragment thereof. In some embodiments, the H1 promoter comprises or consists of the nucleic acid sequence of SEQ ID NO: 122, or a biologically active fragment thereof.

In certain aspects, the disclosure also relates to a vector (e.g., a DNA vector) comprising the polynucleotide encoding one or more gRNAs as described herein. In some embodiments, the vector is a recombinat adenovirus.

### Adenovirus

In certain aspects, the disclosure relates to an adenovirus comprising a polynucleotide encoding one or more gRNAs as described herein. Suitable adenoviruses are described, for example, in U.S. Pat. No. 11,090,344, which is incorporated by reference herein in its entirety.

Direct injection of adenoviruses into lung tumors has been a routine procedure in clinical trials evaluating gene therapy of lung cancer. Dong et al., J. Int. Med. Res. 36, 1273-1287 (2008); Li et al., Cancer Gene Ther. 20, 251-259 (2013); Zhou, et al., Cancer Gene Ther. 23, 1-6 (2016). Adenoviruses can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the adenovirus by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the adenovirus. Such components can be provided as a natural feature of the adenovirus, or the adenovirus can be modified to provide such functionalities.

In some embodiments, the adenovirus is a helper-dependent adenovirus. In some embodiments, the adenovirus is replication competent. In some embodiments, the adenovirus is replication incompetent. Replication incompetent recombinant adenoviral vectors can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Several features of adenoviruses make them suitable vehicles for transferring genetic material to cells for therapeutic applications. For example, adenoviruses can be produced in high titers and can transfer genetic material to nonreplicating and replicating cells. The adenoviral genome can be manipulated to carry a large amount of exogenous DNA (up to about 8 kb or more), and the adenoviral capsid can potentiate the transfer of even longer sequences (Curiel et al., Hum. Gene Ther., 3:147-154 (1992)).

In some embodiments, the adenovirus is a human adenovirus. Human adenovirus (HAd) is a large (about 36 kb) DNA virus that infects humans and contains a double-stranded, linear DNA genome. There are approximately 57 serotypes of human adenovirus, which are divided into six families based on molecular, immunological, and functional criteria. Adenoviral infection of host cells results in adenoviral DNA being maintained episomally, which reduces the potential genotoxicity associated with integrating vectors. Also, adenoviruses are structurally stable, and no genome rearrangement has been detected after extensive amplification. Adenovirus can infect most epithelial cells regardless of their cell cycle stage. So far, adenoviral infection appears to be linked only to mild disease such as acute respiratory disease in humans.

Members of any of the 57 human adenovirus serotypes (HAdV-1 to 57) may incorporate one or more heterologous nucleic acids as described herein. In some embodiments, the adenovirus is human adenovirus serotype 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or 57. Human adenovirus serotype 5 (Ad5) is well characterized genetically and biochemically (GenBank M73260; AC_000008). Thus, in some embodiments, the adenovirus is an Ad5 serotype or a hybrid serotype comprising an Ad5 component (e.g., Ad5/F35). The adenovirus may be a wild type strain but is preferably genetically modified to enhance tumor selectivity, for example by attenuating the ability of the virus to replicate within normal quiescent cells without affecting the ability of the virus to replicate in tumor cells. Non-limiting examples of replication competent adenoviruses encompassed by the present invention include Delta-24, Delta-24-RGD, ICOVIR-5, ICOVIR-7, ONYX-015, ColoAd1, H101 and AD5/3-D24-GMCSF. Onyx-015 is a hybrid of virus serotype Ad2 and Ad5 with deletions in the E1B-55K and E3B regions to enhance cancer selectivity. H101 is a modified version of Onyx-015. ICOVIR-5 and ICOVIR-7 comprise an Rb-binding site deletion of E1A and a replacement of the E1A promoter by an E2F promoter. ColoAd1 is a chimeric Add11p/Ad3 serotype. AD5/3-D24-CFMCSF (CGTG-102) is a serotype 5/3 capsid-modified adenovirus encoding GM-CSF (the Ad5 capsid protein knob is replaced with a knob domain from serotype 3).

In some embodiments, the adenovirus is Delta-24 or Delta-24-RGD. Delta-24 is described in U.S. Patent Application Publication Nos. 20030138405, and 20060147420, each of which are incorporated herein by reference. The Delta-24 adenovirus is derived from adenovirus type 5 (Ad-5) and contains a 24-base-pair deletion within the CR2 portion of the ELA gene that encompasses the area responsible for binding Rb protein (nucleotides 923-946) corresponding to amino acids 122-129 in the encoded E1A protein (Fueyo J et al., Oncogene, 19:2-12 (2000)). Delta-24-RGD further comprises an insertion of the RGD-4C sequence (which hinds strongly to .alpha.v.beta.3 and .alpha.v.beta.5 integrins) into the H1 loop of the fiber knob protein (Pasqualini R. et al., Nat Biotechnol, 15:542-546 (1997)). The E1A deletion increases the selectivity of the virus for cancer cells; the RGD-4C sequence increases the infectivity of the virus in gliomas.

In some embodiments, the adenovirus comprises at least one modified capsid protein. The adenovirus capsid mediates the key interactions of the early stages of the infection of a cell by the virus and is required for packaging adenovirus genomes at the end of the adenovirus life cycle. The capsid comprises 252 capsomeres, which includes 240 hexons, 12 penton base proteins, and 12 fibers (Ginsberg et al., Virology, 28:782-83 (1966)). In one embodiment, one or more capsid proteins (also referred to herein as “coat” proteins) of the adenovirus or adenoviral vector can be manipulated to alter the binding specificity or recognition of the virus or vector for a receptor on a potential host cell. It is well known in the art that almost immediately after intravenous administration, adenovirus vectors are predominantly sequestered by the liver, with clearance of Ad5 from the bloodstream and accumulation in the liver occurring within minutes of administration (Alemany et al., J. Gen. Virol., 81:2605-2609 (2000)). Liver sequestration of adenovirus is primarily due to the abundance of the native coxsackie and adenovirus receptor (CAR) on hepatocytes. Thus, the manipulation of capsid proteins may broaden the range of cells infected by the adenovirus or adenoviral vector or enable targeting of the adenoviral vector to a specific cell type. For example, one or more capsid proteins may be manipulated so as to target the adenovirus to tumor cells or tumor-associated cells. Such manipulations can include deletions of the fiber, hexon, and/or penton proteins (in whole or in part), insertions of various native or non-native ligands into portions of the capsid proteins, and the like.

In some embodiments, the adenovirus comprises a modified fiber protein. The adenovirus fiber protein is a homotrimer of the adenoviral polypeptide IV that has three domains: the tail, shaft, and knob. (Devaux et al., J. Molec. Biol., 215:567-88 (1990), Ych et al., Virus Res., 33:179-98 (1991)). The fiber protein mediates primary viral binding to receptors on the cell surface via the knob and the shaft domains (Henry et al., J. Virol., 68 (8): 5239-46 (1994)). The amino acid sequences for trimerization are located in the knob, which appears necessary for the amino terminus of the fiber (the tail) to properly associate with the penton base (Novelli et al., Virology, 185:365-76 (1991)). In addition to recognizing cell receptors and binding the penton base, the fiber contributes to serotype identity. Fiber proteins from different adenoviral serotypes differ considerably (see, e.g., Green et al., EMBO J., 2:1357-65 (1983), Chroboczek et al., Virology, 186:280-85 (1992), and Signas et al., J. Virol., 53:672-78 (1985)). Thus, the fiber protein has multiple functions key to the life cycle of adenovirus.

In some embodiments, the adenovirus is replication incompetent. Replication incompetent adenoviruses are described, for example, in US20180208944, which is incorporated by reference herein in its entirety. The replication incompetent adenovirus may retain at least a portion of the adenoviral genome. The adenovirus can comprise a nucleic acid sequence that encodes any suitable adenovirus protein, such as, for example, a protein encoded by any one of the early region genes (i.e., E1A, E1B, E2A, E2B, E3, and/or E4 regions), or a protein encoded by any one of the late region genes, which encode the virus structural proteins (i.e., L1, L2, L3, L4, and L5 regions). Alternatively, the adenovirus can comprise non-protein coding regions of the adenoviral genome, including, for example one or more promoters, transcription termination sequences, or polyadenylation sequences of early region or late region genes, or one or more inverted terminal repeat (ITR) sequences.

The replication incompetent adenovirus can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad)). See Morsy et al., Proc. Natl. Acad. Sci. USA, 95:965-976 (1998); Chen et al., Proc. Natl. Acad. Sci. USA, 94:1645-1650 (1997); and Kochanek et al., Hum. Gene Ther., 10:2451-2459 (1999). Examples of replication incompetent adenovirues are disclosed in U.S. Pat. Nos. 5,837,511; 5,851,806; 5,994,106; 6,127,175; 6,482,616; and 7,195,896, and International Patent Application Publications WO 1994/028152, WO 1995/002697, WO 1995/016772, WO 1995/034671, WO 1996/022378, WO 1997/012986, WO 1997/021826, and WO 2003/022311.

The early regions of the adenoviral genome include the E1, E2, E3, and E4 regions. The E1 region comprises the E1A and E1B subregions, and one or more deficiencies in replication-essential gene functions in the E1 region can include one or more deficiencies in replication-essential gene functions in either or both of the E1A and E1B subregions, thereby requiring complementation of the E1A subregion and/or the E1B subregion of the adenoviral genome for the adenovirus to propagate (e.g., to form adenoviral vector particles). The E2 region comprises the E2A and E2B subregions, and one or more deficiencies in replication-essential gene functions in the E2 region can include one or more deficiencies in replication-essential gene functions in either or both of the E2A and E2B subregions, thereby requiring complementation of the E2A subregion and/or the E2B subregion of the adenoviral genome for the adenovirus to propagate (e.g., to form adenovirus particles).

The E3 region does not include any replication-essential gene functions, such that a deletion of the E3 region in part or in whole does not require complementation of any gene functions in the E3 region for the adenovirus to propagate (e.g., to form adenovirus particles). The E3 region may be deleted in whole or in part, or retained in whole or in part.

The E4 region comprises multiple open reading frames (ORFs) that encode multiple endogenous genes. An adenovirus with a deletion of all of the open reading frames of the E4 region except ORF6, and in some cases ORF3, does not require complementation of any gene functions in the E4 region for the adenovirus to propagate.

The late regions of the adenoviral genome include the L1, L2, L3, L4, and L5 regions. The adenovirus also can have a mutation in the major late promoter (MLP), as discussed in International Patent Application Publication WO 2000/000628, which can render the adenovirus replication incompetent.

By removing all or part of the adenoviral genome, for example, one or more endogenous nucleotides of the E1, E3, and E4 regions of the adenoviral genome, the resulting adenovirus is able to accept inserts of exogenous nucleic acid sequences while retaining the ability to be packaged into adenoviral capsids. An exogenous nucleic acid sequence can be inserted at any position in the adenoviral genome so long as insertion in the position allows for the formation of the adenovirus particle. In some embodiments, the exogenous nucleic acid sequence is positioned in the E1 region, the E3 region, or the E4 region of the adenoviral genome.

In some embodiments, the adenovirus is a simian adenovirus, for example, from chimpanzees (Pan troglodytes), bonobos (Pan paniscus), gorillas (Gorilla gorilla) or orangutans (Pongo abelii and Pongo pygnaeus). In a particular embodiment, the adenovirus is a chimpanzee adenovirus. Chimpanzee adenoviruses include, but are not limited to AdY25, ChAd3, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155, ChAd15, SadV41, sAd4310A, sAd4312, SAdV31, SAdV-A1337, ChAdOx1, ChAdOx2 and ChAd157.

Adenoviruses injected into a tumor induce cell death and release of new adenovirus progeny that, by infecting the neighbor cells, generates a treatment wave that, if not halted, may lead to the total destruction of the tumor. Significant antitumor effects of Delta-24 have been shown in cell culture systems and in malignant glioma xenograft models. Delta-24-RGD has shown surprising anti-tumor effects in clinical trials. Although lysis of tumor cells is the main anti-cancer mechanism proposed for Delta-24-RGD oncolytic adenovirus, data from the Phase 1 clinical trial in patients with recurrent glioma and other observations indicate that the direct oncolytic effect may be enhanced by the adenovirus-mediated trigger of anti-tumor immune response.

The infectious cycle of the adenovirus takes place in 2 steps: the early phase which precedes initiation of the replication of the adenoviral genome, and which permits production of the regulatory proteins and proteins involved in the replication and transcription of the viral DNA, and the late phase which leads to the synthesis of the structural proteins. The early genes are distributed in 4 regions that are dispersed in the adenoviral genome, designated E1 to E4 (E denotes “early”). The early regions comprise at least-six transcription units, each of which possesses its own promoter. The expression of the early genes is itself regulated, some genes being expressed before others. Three regions, E1, E2, and E4 are essential to replication of the virus. Thus, if an adenovirus is defective for one of these functions this protein will have to be supplied in trans, or the virus cannot replicate.

The E1 early region is located at the 5′ end of the adenoviral genome, and contains 2 viral transcription units, E1A and E1B. This region encodes proteins that participate very early in the viral cycle and are essential to the expression of almost all the other genes of the adenovirus. In particular, the E1A transcription unit codes for a protein that transactivates the transcription of the other viral genes, inducing transcription from the promoters of the E1B, E2A, E2B, E3, E4 regions and the late genes. Typically, exogenous sequences are integrated in place of all or part of the E3 region.

The adenovirus enters the permissive host cell via a cell surface receptor, and it is then internalized. The viral DNA associated with certain viral proteins needed for the first steps of the replication cycle enters the nucleus of the infected cells, where transcription is initiated. Replication of the adenoviral DNA takes place in the nucleus of the infected cells and does not require cell replication. New viral particles or virions are assembled after which they are released from the infected cells, and can infect other permissive cells.

The adenovirus may be prepared by a suspension cell process. The process can be free of or essentially free of protein, serum, and animal derived components making it suitable for a broad range of both prophylactic and therapeutic products.

If an adenovirus has been mutated so that it is conditionally replicative (i.e., replication-competent under certain conditions), a helper cell may be required for viral replication. When required, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, for example Vero cells or other monkey embryonic mesenchymal or epithelial cells. In certain aspects a helper cell line is 293. Various methods of culturing host and helper cells may be found in the art, for example Racher et al., 1995.

In certain embodiments, the adenovirus is replication-competent in cells with a mutant Rb pathway. After transfection, adenoviral plaques are isolated from the agarose-overlaid cells and the viral particles are expanded for analysis. For detailed protocols the skilled artisan is referred to Graham and Prevac, 1991.

Alternative technologies for the generation of adenovirus vectors include utilization of the bacterial artificial chromosome (BAC) system, in vivo bacterial recombination in a recA+ bacterial strain utilizing two plasmids containing complementary adenoviral sequences, and the yeast artificial chromosome (YAC) system (PCT publications WO1995/27071 and WO1996/33280, which are incorporated herein by reference).

Adenovirus is easy to grow and manipulate and exhibits broad host range in vitro and in vivo. This group of viruses can be obtained in high titers and they are highly infective. The life cycle of adenovirus does not require integration into the host cell genome.

Modifications of the adenoviruses described herein may be made to improve the ability of the oncolytic adenovirus to treat cancer. Such modifications of an oncolytic adenovirus have been described by Jiang et al. (Curr Gene Ther. 2009 Oct. 9 (5): 422-427), see also U.S. patent application No. 20060147420, each of which are incorporated herein by reference.

The absence or the presence of low levels of the coxsackievirus and adenovirus receptor (CAR) on several tumor types can limit the efficacy of the adenovirus. Various peptide motifs may be added to the fiber knob, for instance an RGD motif (RGD sequences mimic the normal ligands of cell surface integrins), Tat motif, polylysine motif, NGR motif, CTT motif, CNGRL motif, CPRECES motif or a strept-tag motif (Rouslahti and Rajotte, 2000). A motif can be inserted into the HI loop of the adenovirus fiber protein. Modifying the capsid allows CAR independent target cell infection. This allows higher replication, more efficient infection, and increased lysis of tumor cells (Suzuki et al., 2001, incorporated herein by reference). Peptide sequences that bind specific human glioma receptors such as EGFR or uPR may also be added. Specific receptors found exclusively or preferentially on the surface of cancer cells may be used as a target for adenoviral binding and infection, such as EGFRvIII.

The adenovirus may further comprise one or more nucleic acid sequences that encode a Clustered Regularly Interspaced Short Palindromic Repeat (CRISPR)-associated endonuclease protein, or a fragment thereof. In some embodiments, the CRISPR-associated endonuclease is a class 2 CRISPR-associated endonuclease. In some embodiments, the class 2 CRISPR-associated endonuclease is Cas9 or Cas12a. In a particular embodiment, the nucleic acid that encodes the CRISPR-associated endonuclease or fragment thereof is operably linked to a promoter selected from the group consisting of a tissue-specific promoter, an H1 promoter, a minimal cytomegalovirus (miniCMV) promoter and an elongation factor 1α short (EFS) promoter. In some embodiments, the miniCMV promoter comprises or consists of the nucleic acid sequence of SEQ ID NO: 116. In some embodiments, the EFS promoter comprises of consists of the nucleic acid sequence of SEQ ID NO: 117.

In some embodiments, the same H1 promoter is used to drive expression of the CRISPR-associated endonuclease and one or more sgRNAs. See Gao et al., 2018, Molecular Therapy: Nucleic Acids 14:32-40, which is incorporated by reference herein in its entirety. For example, in some embodiments, a single H1 promoter is operably linked to both a nucleic acid that encodes a CRISPR-associated endonuclease and a nucleic acid that encodes one or more sgRNAs.

In some embodiments, the nucleic acid sequence that encodes the CRISPR-associated endonuclease or fragment thereof is operably linked to at least one nucleus localization signal (NLS). For example, in some embodiments, the nucleic acid sequence that encodes the CRISPR-associated endonuclease or fragment thereof is operably linked to 1, 2, 3, 4 or 5 nucleus localization signals. In some embodiments, the NLS is an SV40 NLS. In some embodiments, the SV40 NLS comprises or consists of the nucleic acid sequence of SEQ ID NO: 121.

In some embodiments, the nucleic acid that encodes the CRISPR-associated endonuclease or fragment thereof and the one or more DNA sequences encoding the gRNA are on the same vector. In some embodiments, the nucleic acid that encodes the CRISPR-associated endonuclease or fragment thereof and the one or more DNA sequences encoding the gRNA are on separate vectors.

In some embodiments, the entire nucleic acid sequence encoding CRISPR-associated endonuclease (e.g. Cas 9) is on one vector. However, a major limitation to their application in gene therapy is the size of some CRISPR-associated endonucleases such as Cas9 (>4 kb), impeding its efficient delivery. Accordingly, the nucleic acid sequence encoding the CRISPR-associated endonuclease may be divided into two fragments and placed on separate vectors. In some embodiments, the two fragments of the CRISPR-associated endonuclease (e.g. Cas 9) are ligated together by using a split-intein system. An intein is an intervening protein domain that undergoes a unique posttranslational autoprocessing event, termed protein splicing. In this spontaneous process, the intein excises itself from the host protein and, in the process, ligates together the flanking N- and C-terminal residues (exteins) to form a native peptide bond. Each half of the CRISPR-associated endonuclease (e.g. Cas 9) is fused to a split-intein moiety, and upon co-expression intein-mediated transsplicing occurs and the full CRISPR-associated endonuclease is reconstituted. Split-intein systems for expressing CRISPR-associated endonucleases are known in the art and are described, for example, in Truong et al., 2015, Nucleic Acids Research 43 (13): 6450-6458, and Schmelas et al., 2018, Biotechnology Journal 13:1700432, each of which is incorporated by reference herein in its entirety. Alternatively, the CRISPR-associated endonuclease (e.g. Cas9) can be split into two fragments and rendered chemically inducible by rapamycin sensitive dimerization domains for controlled reassembly. See Zetche et al., 2015, Nat Biotechnol. February; 33 (2): 139-142, which is incorporated by reference herein in its entirety.

### Pharmaceutical Compositions

Any of the pharmaceutical compositions disclosed herein can be formulated for use in the preparation of a medicament, and particular uses are indicated below in the context of treatment, e.g., the treatment of a subject having cancer. When employed as pharmaceuticals, any of the nucleic acids and vectors can be administered in the form of pharmaceutical compositions. Administration may be pulmonary (e.g., by inhalation or insufflation of powders or aerosols, including by nebulizer; intratracheal, intranasal, epidermal and transdermal), topical (including ophthalmic and to mucous membranes including intranasal, vaginal and rectal delivery), ocular, oral or parenteral. Parenteral administration includes intravenous, intra-arterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

In some embodiments, pharmaceutical compositions can contain, as the active ingredient, nucleic acids and vectors described herein in combination with one or more pharmaceutically acceptable carriers. The term “pharmaceutically acceptable” refers to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the pharmaceutical compositions disclosed herein, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The adenoviruses disclosed herein can be delivered to an appropriate cell of a subject, e.g. a cancer cell. This can be achieved by, for example, the use of a polymeric, biodegradable microparticle or microcapsule delivery vehicle, sized to optimize phagocytosis by phagocytic cells such as macrophages.

In some embodiments, the pharmaceutical compositions can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

The adenoviruses may also be applied to a surface of a device (e.g., a catheter) or contained within a pump, patch, or other drug delivery device. The adenoviruses disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions comprising the adenoviruses can be formulated as a nanoparticle encapsulating the adenovirus.

In another aspect, the present disclosure provides a method for delivery of a polynucleotide encoding one or more gRNA to a subject that involves transfecting or infecting a selected host cell (e.g. a cancer cell) of the subject with an adenovirus comprising the polynucleotide. This method involves transfecting or infecting a selected host cell with an adenovirus containing a polynucleotide encoding one or more sgRNA under the control of sequences that direct expression thereof. The adenovirus may be delivered in a pharmaceutical composition comprising a pharmaceutically acceptable carrier. The adenovirus may be administered to a human or non-human mammalian subject.

The adenovirues are administered in sufficient amounts to transfect the target cells (e.g. cancer cells) and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the lung), oral, inhalation, intranasal, intratracheal, intraarterial, intraocular, intravenous, intramuscular, subcutaneous, intradermal, and other parental routes of administration. Routes of administration may be combined, if desired. In a particular embodiment, the route of administration is intratumoral.

Dosages of the adenovirus will depend primarily on factors such as the condition being treated, the age, weight and health of the patient, and may thus vary among patients. For example, a therapeutically effective human dosage of the adenovirus is generally in the range of from about 0.01 mL to about 100 mL of solution containing concentrations of from about 1×10⁶ to 1×10¹⁶ genomes virus vector. For example, in some embodiments the therapeutically effective human dosage of the adenovirus is 0.01 mL to 10 mL, 0.1 mL to 10 mL or 0.1 mL to 100 mL. In some embodiments, the solution concentration is 1×10⁶ to 1×10¹², or 1×10⁶ to 1×10¹⁴ genomes virus vector. The dosage will be adjusted to balance the therapeutic benefit against any side effects and such dosages may vary depending upon the therapeutic application for which the recombinant vector is employed. The levels of expression of the transgene can be monitored to determine the frequency of dosage.

### Methods of Reducing Gene Expression or Activity in a Cell

In certain aspects the disclosure relates to a method of reducing expression or activity of a gene in a cancer cell comprising contacting the cancer cell with an adenovirus encoding one or more gRNAs as described herein, or a pharmaceutical composition comprising an adenovirus encoding one or more gRNAs as described herein, wherein the gRNA hybridizes to the gene and a CRISPR-associated endonuclease cleaves the gene, and wherein expression or activity of the gene is reduced in the cancer cell relative to a cancer cell in which the adenovirus is not introduced.

In certain aspects the disclosure relates to a method of reducing expression or activity of a gene in a cancer cell in a subject, the method comprising administering to the subject an effective amount of a pharmaceutical composition comprising an adenovirus encoding one or more gRNAs as described herein to the subject, wherein the gRNA hybridizes to the gene and a CRISPR-associated endonuclease cleaves the gene, and wherein expression or activity of the gene is reduced in the cancer cell in the subject relative to a cancer cell in a subject that is not administered the pharmaceutical composition.

In some embodiments, reducing expression or activity of a gene comprises reducing transcription of RNA (e.g., an mRNA) encoded by the gene. In some embodiments, reducing expression or activity of a gene comprises reducing the level and/or activity of a polypeptide encoded by the gene.

In some embodiments, the gene is an NRF2 gene. For example, in certain aspects, the disclosure relates to a method of reducing NRF2 expression or activity in a cancer cell comprising introducing into the cancer cell an adenovirus comprising a polynucleotide comprising: (a) a first DNA sequence encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in an NRF2 gene; and (b) a first promoter that is operably linked to the DNA sequence, wherein the gRNA hybridizes to the NRF2 gene and a CRISPR-associated endonuclease cleaves the NRF2 gene, and wherein NRF2 expression or activity is reduced in the cancer cell relative to a cancer cell in which the adenovirus is not introduced.

In certain aspects, the disclosure relates to a method of reducing NRF2 expression or activity in a cancer cell in a subject, the method comprising administering to the subject an effective amount of an adenovirus comprising a polynucleotide comprising: (a) a first DNA sequence encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in an NRF2 gene; and (b) a first promoter that is operably linked to the DNA sequence, or a pharmaceutical composition comprising the adenovirus, wherein the gRNA hybridizes to the NRF2 gene and a CRISPR-associated endonuclease cleaves the NRF2 gene, and wherein NRF2 expression or activity is reduced in the cancer cell in the subject relative to a cancer cell in a subject that is not administered the adenovirus or pharmaceutical composition.

Reducing NRF2 expression in the cell may comprise reducing expression of NRF2 mRNA in the cell, reducing expression of the NRF2 protein in the cell, or both. Because the ploidy level of a cancer cell may change from the normal diploid, the cancer cell may comprise 1, 2, 3, 4, 5 or more NRF2 alleles. Accordingly, in some embodiments, expression of at least 1, 2, 3, 4 or 5 alleles of the NRF2 gene is reduced. In some embodiments, expression of all alleles of the NRF2 gene is reduced. In some embodiments, introducing the polynucleotide encoding the gRNA and the nucleic acid sequence encoding a CRISPR-associated endonuclease into the cell reduces NRF2 expression and/or activity in the cell, but does not completely eliminate it. In other embodiments, NRF2 expression and/or activity in the cell are completely eliminated.

The gRNA may be complementary to a target sequence in an exon of the NRF2 gene. In a particular embodiment, the gRNA is complementary to a target sequence in exon 1, 2, 3, 4, or 5 of the NRF2 gene. In some embodiments, the gRNA is encoded by a single DNA sequence. In other embodiments, the gRNA is encoded by two or more DNA sequences. For example, in some embodiments, the gRNA is encoded by a first DNA sequence encoding a trans-activated small RNA (tracrRNA) and a second D NA sequence encoding a CRISPR RNA (crRNA). The tracrRNA and crRNA may hybridize within the cell to form the guide RNA. Accordingly, in some embodiments, the gRNA comprises a trans-activated small RNA (tracrRNA) and a CRISPR RNA (crRNA).

In some embodiments, the guide RNA is complementary to a variant NRF2 gene that is found only in cancer cells and not in wild-type NRF2 genes in normal (i.e., non-cancerous) cells (e.g. exon 1, 2, 3, 4, or 5). In some embodiments, the variant NRF2 gene comprises an R34G substitution in exon 2. In some embodiments, the guide RNA is complementary to a sequence in exon 2 of a variant NRF2 gene that is found only in cancer cells (e.g. lung cancer cells). In some embodiments, introducing the polynucleotide encoding the gRNA and the nucleic acid sequence encoding a CRISPR-associated endonuclease into the cell reduces variant NRF2 expression and/or activity in the cell, but does not completely eliminate it. In other embodiments, variant NRF2 expression and/or activity in the cell are completely eliminated.

In some embodiments, CRISPR-associated endonucleases suitable for use in reducing expression of the variant NRF2 gene include, but are not limited to, a class 1 CRISPR-associated endonucleases such as, e.g., Cas7 and Cas5, along with, in some embodiments, SS (Cas11) and Cas8a1; Cas8b1; Cas8c; Cas8u2 and Cas6; Cas3″ and Cas10d; Cas SS (Cas11), Cas8c, and Cas6; Cas8f and Cas6f; Cas6f; Cas8-like (Csf1); SS (Cas11) and Cas8-like (Csf1); or SS (Cas11) and Cas10. Class 2 CRISPR-associated endonucleases include type I, type V, and type VI CRISPR-Cas systems, which have a single effector molecule. In some embodiments, CRISPR-associated endonucleases suitable for use in reducing expression of the variant NRF2 gene include, but are not limited to, class 2 CRISPR-associated endonucleases such as, e.g., Cas9, Cas12a, Cas12b, Cas12c, Cas12d, Cas13a, Cas13b, Cas13c, c2c4, c2c5, c2c8, c2c9, and/or c2c10. In some embodiments, CRISPR-associated endonucleases suitable for use in reducing expression of the variant NRF2 gene include, but are not limited to, CasX, CasY, and/or MAD6 (see, e.g., Liu et al., Nature 566:218-23 (2019)).

Any cell containing an NRF2 gene (e.g. a variant NRF2 gene) may be suitable for use in the methods of reducing NRF2 expression or activity described herein. In some embodiments, the cell is a eukaryotic cell, e.g. a mammalian cell. In some embodiments, the cell is a human cell. In some embodiments, the NRF2 gene is a human NRF2 gene.

In certain aspects, the disclosure also relates to a cell comprising a mutated NRF2 gene produced by the methods of reducing NRF2 expression or activity described herein. In some embodiments, the mutated NRF2 gene comprises an insertion or a deletion relative to the endogenous NRF2 gene. In some embodiments, the insertion or deletion occurs within 1, 2, 3, 4, 5, 6, 7, 8, 9, 10, 11, 12, 13, 14, 15, 16, 17, 18, 19 or 20 nucleotide(s) of a protospacer adjacent motif sequence (PAM) in the NRF2 gene.

### Methods for Treatment of Cancer

In certain aspects, the disclosure relates to a method of treating cancer in a subject, the method comprising administering to the subject a therapeutically effective amount of a pharmaceutical composition comprising an adenovirus as described herein to the subject. In some embodiments, the adenovirus comprises a polynucleotide encoding a gRNA comprising a DNA-binding domain that is complementary to a gene selected from the group consisting of NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7. BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNXIT1, DNMT3A, SMCIA, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST, TSC2, EEF1A1, WHSC1, APOB, NUP133, AXIN1, PHF6, TET2, WT1, FLT3, FLT4, SMC3, CEBPA, RAD21, RAD50, RAD51, PTPDC1, ASXL1, EZH2, NPM1, SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA, LATS1, POLRMT, CDK4, COL5A1, PPP6C, MECOM, DACH1, MAP2K1, MAP2K2, RQCD1, DDX3X, NUP93, PPMID, CHD2, CHD3, CCND1, CCND2, CCND3, ACVR1, KMT2A, KMT2B, KMT2C, KMT2D, SIN3A, SCAF4, DICER1, FOXA2, CTNND1, MYC, MYCL, MYCN, SOX17, ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF2I, RIT1, MGA, ABL1, BRAF, CHEK1, FANCC, JAK2, MITF, PDCDILG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSFIR, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKBIA, PRDM1, DOT1L, FGFR1, LMO1, NKX2-1, PREX2, HSD3B1, LRP1B, TSHR, ATRX, CDC73, HSP90AA1, PRKCI, AURKA, PRKDC, VEGFA, AURKB, CDK12, FH, MAGI2, PRSS8, SMO, FLCN, IGFIR, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B, PARK2, RANBP2, SRC, IRS2, PAX5, RARA, BLM, FANCA, JAK1, FCRL4, LIG4, MAR, PWWP3A, MUC16, MUC17, FCGBP, FAT17, MMSET, IRTA2, TTN, DST, and STAT3,

In some embodiments, the gRNA comprises a DNA-binding domain that is complementary to an NRF2 gene. For example, in certain aspects, the disclosure relates to a method of treating cancer in a subject, the method comprising administering to the subject a therapeutically effective amount of am adenovirus comprising a polynucleotide comprising: (a) a first DNA sequence encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in an NRF2 gene; and (b) a first promoter that is operably linked to the DNA sequence, or an adenovirus or pharmaceutical composition comprising the adenovirus as described herein, to the subject.

In some embodiments, the guide RNA is complementary to a variant NRF2 gene that is found only in cancer cells and not in wild-type NRF2 genes in normal (i.e., non-cancerous) cells (e.g. exon 1, 2, 3, 4, or 5). In some embodiments, the guide RNA is complementary to a sequence in exon 2 of a variant NRF2 gene that is found only in cancer cells.

In certain embodiments, the cancer is a solid tumor. In certain embodiments, the cancer is lung cancer. In certain embodiments, the lung cancer is non-small-cell lung cancer (NSCLC). In certain embodiments, the NSCLC is squamous-cell lung carcinoma. In certain embodiments, the cancer is treated only with the pharmaceutical composition comprising a polynucleotide comprising: (a) a first DNA sequence encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in an NRF2 gene; and (b) a first promoter that is operably linked to the DNA sequence. In some embodiments, the guide RNA is complementary to a variant NRF2 gene that is found only in cancer cells (e.g. lung cancer cells) and not to a wild-type NRF2 genes in normal (i.e., non-cancerous) cells (e.g. exon 1, 2, 3, 4, or 5 of the NRF gene). In some embodiments, the guide RNA is complementary to a sequence in exon 2 of a variant NRF2 gene that is found only in cancer cells. In some embodiments, expression or activity of wild-type NRF2 in a non-cancerous cell of the subject is unaffected by administration of the polynucleotide, adenovirus or pharmaceutical composition.

In certain embodiments, the cancer is treated with the pharmaceutical compositions as described herein and an additional agent, e.g. one or more chemotherapeutic agents. In certain embodiments, treatment with the chemotherapeutic agent is initiated at the same time as treatment with the pharmaceutical composition. In certain embodiments, the treatment with the chemotherapeutic agent is initiated after the treatment with the pharmaceutical composition is initiated. In certain embodiments, treatment with the chemotherapeutic agent is initiated at before the treatment with the pharmaceutical composition. In some embodiments, the cancer is resistant to one or more chemotherapeutic agents. The one or more chemotherapeutic agents include, but are not limited to, cisplatin, vinorelbine, carboplatin, paclitaxel, docetaxel, cabazitaxel and combinations thereof.

In certain embodiments, the pharmaceutical compositions of the present disclosure may be utilized for the treatment of cancer wherein the subject has failed at least one prior chemotherapeutic regimen. For example, in some embodiments, the cancer is resistant to one or more chemotherapeutic agents, e. g. cisplatin, vinorelbine, carboplatin, paclitaxel, docetaxel, or cabazitaxel. Accordingly, the present disclosure provides methods of treating cancer in a subject, wherein the subject has failed at least one prior chemotherapeutic regimen for the cancer, comprising administering the pharmaceutical compositions as described herein to the subject in an amount sufficient to treat the cancer, thereby treating the cancer. The pharmaceutical compositions described herein may also be utilized for inhibiting tumor cell growth in a subject wherein the subject has failed at least one prior chemotherapeutic regimen. Accordingly, the present disclosure further provides methods of inhibiting tumor cell growth in a subject, e.g. wherein the subject has failed at least one prior chemotherapeutic regimen, comprising administering the pharmaceutical compositions described herein to the subject, such that tumor cell growth is inhibited. In certain embodiments, the subject is a mammal, e.g. a human.

For example, the pharmaceutical compositions described herein may be administered to a subject in an amount sufficient to reduce proliferation of cancer cells relative to cancer cells that are not treated with the pharmaceutical composition. The pharmaceutical composition may reduce cancer cell proliferation by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to cancer cells that are not treated with the pharmaceutical composition.

In some embodiments, the pharmaceutical composition is administered in an amount sufficient to reduce tumor growth relative to a tumor that is not treated with the pharmaceutical composition. The pharmaceutical composition may reduce tumor growth by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to cancer cells that are not treated with the pharmaceutical composition. In a particular embodiment, administration of the pharmaceutical composition to the subject completely inhibits tumor growth.

In one embodiment, administration of a pharmaceutical composition as described herein, achieves at least stable disease, reduces tumor size, inhibits tumor growth and/or prolongs the survival time of a tumor-bearing subject as compared to an appropriate control. Accordingly, this disclosure also relates to a method of treating tumors in a human or other animal, including a subject, who has failed at least one prior chemotherapeutic regimen, by administering to such human or animal an effective amount of a pharmaceutical composition described herein. One skilled in the art would be able, by routine experimentation with the guidance provided herein, to determine what an effective amount of the pharmaceutical composition would be for the purpose of treating malignancies including in a subject who has failed at least one prior chemotherapeutic regimen. For example, a therapeutically active amount of the pharmaceutical composition may vary according to factors such as the disease stage (e.g., stage I versus stage IV), age, sex, medical complications, and weight of the subject, and the ability of the pharmaceutical composition to elicit a desired response in the subject. The dosage regimen may be adjusted to provide the optimum therapeutic response. For example, several divided doses may be administered daily, the dose may be administered by continuous infusion, or the dose may be proportionally reduced as indicated by the exigencies of the therapeutic situation.

In certain embodiments, the methods further include a treatment regimen which includes any one of or a combination of surgery, radiation, chemotherapy, e.g., hormone therapy, antibody therapy, therapy with growth factors, cytokines, and anti-angiogenic therapy.

Cancers for treatment using the methods of the disclosed herein include, for example, all types of cancer or neoplasm or malignant tumors found in mammals, including, but not limited to: leukemias, lymphomas, melanomas, carcinomas and sarcomas. In one embodiment, cancers for treatment using the methods of disclosed herein include melanomas, carcinomas and sarcomas. In some embodiments, the compositions are used for treatment, of various types of solid tumors, for example breast cancer, bladder cancer, colon and rectal cancer, endometrial cancer, kidney (renal cell) cancer, lung cancer, melanoma, pancreatic cancer, prostate cancer, thyroid cancer, skin cancer, bone cancer, brain cancer, cervical cancer, liver cancer, stomach cancer, mouth and oral cancers, neuroblastoma, testicular cancer, uterine cancer, thyroid cancer, head and neck, kidney, lung, non-small cell lung, melanoma, mesothelioma, ovary, sarcoma, stomach, uterus and medulloblastoma, and vulvar cancer. In certain embodiments, solid tumors include breast cancer, including triple negative breast cancer. In certain embodiments, skin cancer includes melanoma, squamous cell carcinoma, cutaneous T-cell lymphoma (CTCL). In certain embodiments, the cancer includes leukemia. In certain embodiments, the cancer is selected from the group consisting of lung cancer, melanoma, esophageal squamous cancer (ESC), head and neck squamous cell carcinoma (HNSCC), and breast cancer.

In a particular embodiment, the cancer is lung cancer, e.g. non-small-cell lung cancer (NSCLC). In some embodiments, the NSCLC is adenocarcinoma, squamous cell carcinoma, or large cell carcinoma. Beyond the current well-established combinatorial drug strategies used to treat NSCLC, several different combinatorial approaches are also being investigated for the treatment of cancers. For example, the use of an oncolytic virus that infects tumor cells has been found to enhance the activity of chemotherapy. Infection with myxoma virus combined with cisplatin or gemcitabine efficiently destroyed ovarian cancer cells at much lower dosages than needed without viral addition. Nounamo et al., Mol. Ther. oncolytics 6, 90-99 (2017). The use of oncolytic virus therapy and cytotoxic chemotherapy for improved effectiveness of cancer treatment is an active area of development. Wennier, et al., Curr. Pharm. Biotechnol. 13, 1817-33 (2012); Pandha, et al., Oncolytic Virotherapy 5, 1 (2016). Infection with a replication competent virus before treatment with cisplatin markedly enhances the therapeutic benefit of chemotherapy.

With the usage of targeted therapy (targeting EGFR mutation, ALK rearrangement, etc.) and immunotherapy (checkpoint inhibitors, anti-PD1, anti-CTLA4, etc.), the clinical management of NSCLC has greatly improved. Patients can have a longer and better quality of life. However, these therapies cannot solve all the problems. For example, agents that target specific molecules typically have a response rate of ˜70%. However, after a median period of 8-16 months, due to the inevitable resistance, relapse happens in almost all patients. Anichini, et al., Cancer Immunol. Immunother. 67, 1011-1022 (2018). In regards to immunotherapy, though pembrolizumab (Keytruda) can be used as first treatment in certain lung cancer patients, only a fraction of them will respond. Bianco, et al., Curr. Opin. Pharmacol. 40, 46-50 (2018).

On the other hand, chemotherapy is still indispensable in the lung cancer treatment paradigm. In patients with locoregional NSCLC, chemotherapy is the only systemic therapy proven to improve curability when combined with surgery or radiation. Wang, et al., Investig. Opthalmology Vis. Sci. 58, 3896 (2017). In patients with metastasis, chemotherapy is still the mainstay of care for those who have developed resistance to targeted therapy agents. Meanwhile, it also has the potential to stimulate the immune system to boost the effectiveness of immunotherapy.

### Combination Therapies

In certain embodiments, the pharmaceutical compositions described herein can be used in combination therapy with at least one additional anticancer agent, e.g., a chemotherapeutic agent. Small molecule chemotherapeutic agents generally belong to various classes including, for example: 1. Topoisomerase II inhibitors (cytotoxic antibiotics), such as the anthracyclines/anthracenediones, e.g., doxorubicin, epirubicin, idarubicin and nemorubicin, the anthraquinones, e.g., mitoxantrone and losoxantrone, and the podophillotoxines, e.g., etoposide and teniposide; 2. Agents that affect microtubule formation (mitotic inhibitors), such as plant alkaloids (e.g., a compound belonging to a family of alkaline, nitrogen-containing molecules derived from plants that are biologically active and cytotoxic), e.g., taxanes, e.g., paclitaxel and docetaxel, and the vinka alkaloids, e.g., vinblastine, vincristine, and vinorelbine, and derivatives of podophyllotoxin; 3. Alkylating agents, such as nitrogen mustards, ethyleneimine compounds, alkyl sulphonates and other compounds with an alkylating action such as nitrosoureas, dacarbazine, cyclophosphamide, ifosfamide and melphalan; 4. Antimetabolites (nucleoside inhibitors), for example, folates, e.g., folic acid, fiuropyrimidines, purine or pyrimidine analogues such as 5-fluorouracil, capecitabine, gemcitabine, methotrexate, and edatrexate; 5. Topoisomerase I inhibitors, such as topotecan, irinotecan, and 9-nitrocamptothecin, camptothecin derivatives, and retinoic acid; and 6. Platinum compounds/complexes, such as cisplatin, oxaliplatin, and carboplatin. Exemplary chemotherapeutic agents for use in the methods of disclosed herein include, but are not limited to, amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-I 1, 10-hydroxy-7-ethyl-camptothecin (SN38), capecitabine, ftorafur, 5′deoxyflurouridine, UFT, eniluracil, deoxycytidine, 5-azacytosine, 5-azadeoxycytosine, allopurinol, 2-chloro adenosine, trimetrexate, aminopterin, methylene-10-deazaaminopterin (MDAM), oxaplatin, picoplatin, tetraplatin, satraplatin, platinum-DACH, ormaplatin, CI-973 (and analogs thereof), JM-216 (and analogs thereof), epirubicin, 9-aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, acyclovir, valacyclovir, ganciclovir, amantadine, rimantadine, lamivudine, zidovudine, bevacizumab, trastuzumab, rituximab, Pentostatin, floxuridine, fludarabine, hydroxyurea, ifosfamide, idarubicin, mesna, irinotecan, mitoxantrone, topotecan, leuprolide, megestrol, melphalan, plicamycin, mitotane, pegaspargase, pipobroman, tamoxifen, teniposide, testolactone, thiotepa, uracil mustard, vinorelbine, chlorambucil, mTor, epidermal growth factor receptor (EGFR), and fibroblast growth factors (FGF) and combinations thereof which are readily apparent to one of skill in the art based on the appropriate standard of care for a particular tumor or cancer. In a particular embodiment, the chemotherapeutic agent is selected from the group consisting of cisplatin, vinorelbine, carboplatin, and combinations thereof (e.g., cisplatin and vinorelbine; cisplatin and carboplatin; vinorelbine and carboplatin; cisplatin, vinorelbine, and carboplatin).

In some embodiments, the pharmaceutical composition is administered in an amount sufficient to reduce tumor growth relative to a tumor that is treated with the at least one chemotherapeutic agent but is not treated with the pharmaceutical composition. The pharmaceutical composition may reduce tumor growth by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to cancer cells that are treated with the at least one chemotherapeutic agent but are not treated with the pharmaceutical composition.

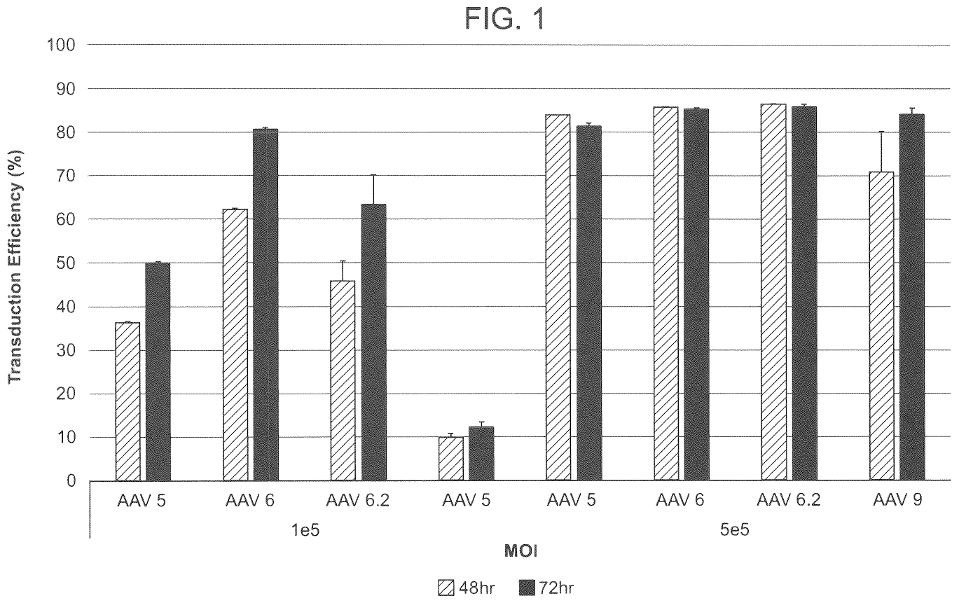

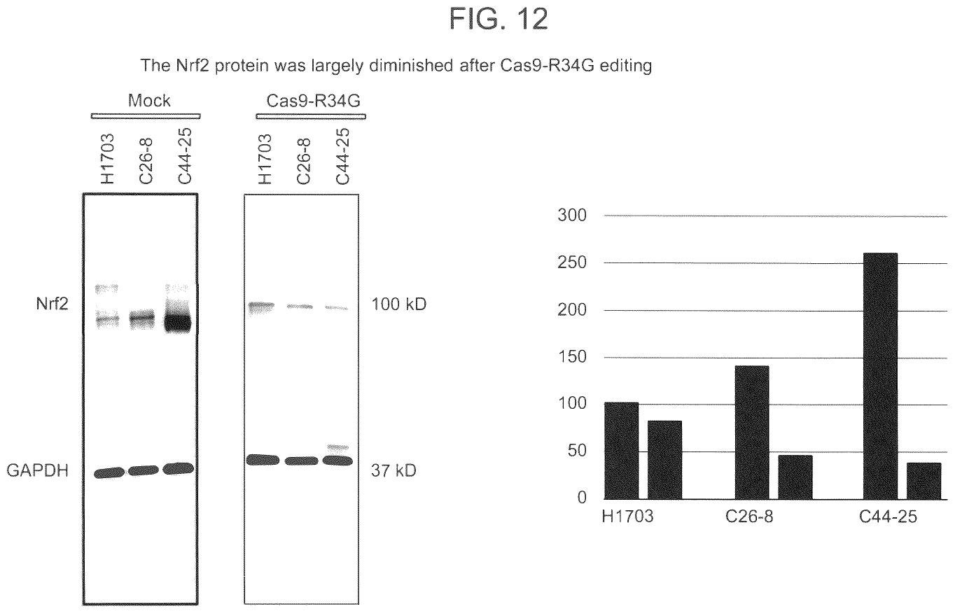

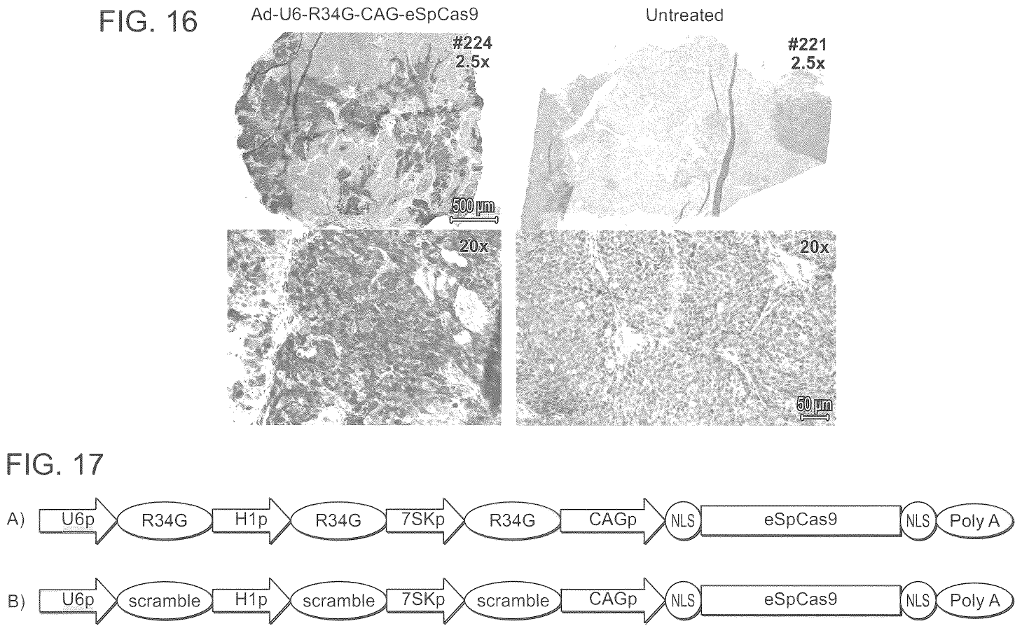

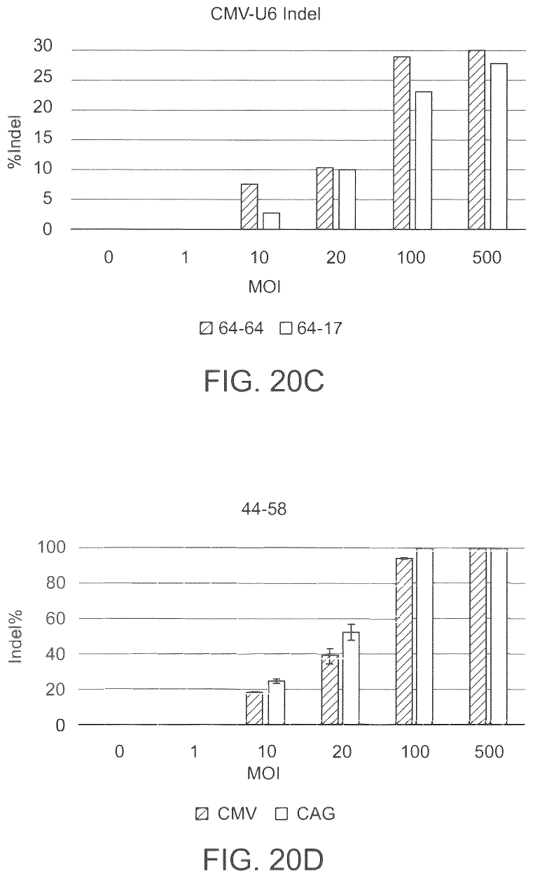

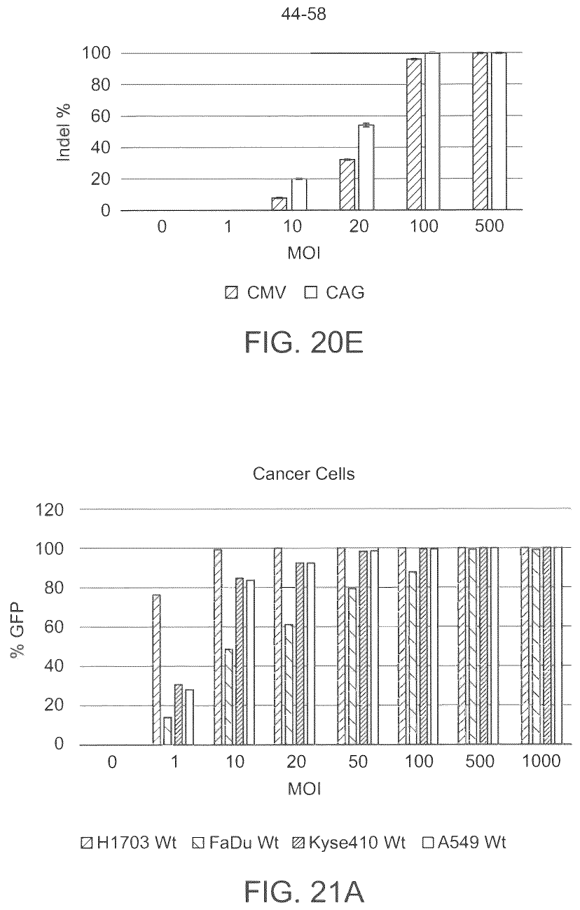

TABLE 3
Description of Sequences. PAM sequences are underlined.

| SEQ ID | | |
|---|---|---|
| NO: | Description | Sequence |
| 1 | Guide RNA1 (gRNA1) | 3′-AGCTACTCTGGCCCTTATAGTCC-5′ |
| | recognition element | |
| 2 | Guide RNA1 (gRNA1) | 5′-UCGAUGUGACCGGGAAUAUCAGG-3′ |
| 3 | Guide RNA2 (gRNA2) | 3′-ACTAAATCTGCCATACGTTGTCC-5′ |
| | recognition element (from Sanjana | |
| | et al., Nat. Methods 11(8):783-84 | |
| | (2014)) | |
| 4 | Guide RNA2 (gRNA2) | 5′-UGAUUUAGACGGUAUGCAACAGG-3′ |
| 5 | PCR primer, forward | GTAGTGGTGCCTTAGAGCTTACTCATCC |
| 6 | PCR primer, reverse | CTAGCATGGGCAGTACTCATGACTAAG |
| 7 | human NRF2 DNA sequence | NCBI Reference Sequence NM_006164.5 |
| 8 | human NRF2 amino acid sequence | NCBI Reference Sequence NP_006155.2 |
| 9 | Exon 4 gRNA1 | |
| 10 | Exon 4 gRNA2 | |
| 11 | Exon 3 gRNA3 | |
| 12 | Exon 3 gRNA4 | |
| 13 | Exon 2 gRNA | |
| 14 | Exon 5 gRNA | |
| 15 | Exon 2 gRNA | |
| 16 | Exon 5 gRNA | |
| 17-40 | Table 2, DNA binding domains of | |
| | cancer-derived gRNAs from | |
| | variant NRF2s | |
| 41-52 | Table 1, DNA sequences encoding | |
| | NRF2 mutations | |
| 53-56 | Table 2, DNA binding domains of | |
| | cancer-derived gRNAs from | |
| | variant NRF2s | |
| 57-58 | Table 1, DNA sequences encoding | |
| | NRF2 mutations | |
| 59-63 | DNA oligos encoding DNA | |
| | binding domains of sgRNAs | |
| 64 | Exon 2 gRNA | |
| 65 | R34G ssDNA template | |
| 66 | Exon 4 gRNA1 | |
| 67 | Exon 4 gRNA2 | |
| 68-77 | FIG. 1A | |
| 78-79 | FIG. 1C | |
| 80-86 | FIG. 12 | |
| 87-91 | FIG. 15 | |
| 92-95 | FIG. 19 | |
| 96-99 | FIG. 20 | |
| 100-102 | FIG. 23 | |
| 103-112 | FIG. 30 | |
| 113 | gRNA scaffold, Cas9 protein- | |
| | binding domain | |
| 114 | Cas9 amino acid sequence | |
| 115 | Cas9 nucleotide sequence | |
| 116 | miniCMV promoter | |
| 117 | EF-1a promoter | |
| 118 | U6 promoter | |
| 119 | CMV promoter | |
| 120 | AAV2 ITR | |
| 121 | SV40 NLS | |
| 122 | H1 promoter | |
| 123 | 7SK promoter | |
| 124 | DNA sequence of full-length | |
| | gRNA, DNA binding domain | |
| | encoded by SEQ ID NO: 61 | |
| 125 | Expression cassette containing H1, | |
| | 7SK and U6 promoters and gRNA | |
| | DNA binding domain of SEQ ID | |
| | NO: 61 | |
| 126 | RNA sequence of DNA binding | |
| | domain encoded by SEQ ID NO: | |
| | 61 | |
| 127 | RNA sequence of gRNA scaffold, | |
| | encoded by SEQ ID NO: 113 | |
| 128 | RNA sequence of full-length | |
| | sgRNA, DNA binding domain | |
| | encoded by SEQ ID NO: 61 | |
| 129 | EGFR DNA sequence | |
| 130 | EGFR amino acid sequence | |
| 131 | Scrambled gRNA | GCACTACCAGAGCTAACTCA |
| 132 | Neh2 forward PCR primer | CACCATCAACAGTGGCATAATGTGAA |
| 133 | Neh2 reverse PCR primer | AACTCAGGTTAGGTACTGAACTCATCA |

### EXAMPLES

### Example 1: AAV Serotype Assessment of AAV5, AAV6, AAV6.2 and AAV9 Using a scAAV-GFP Construct

AAV 5, 6, 6.2 and 9 that express eGFP were purchased from the Horae Gene Therapy Center at University of Massachusetts Medical School. NCIH1703 [H1703] cells were purchased from ATCC (ATCC® CRL¬5889™) and cultured in RPMI 1640 (ATCC formulation) medium supplemented with 10% FBS. H1703 cells were removed from culture flasks and seeded into 12-well cell culture plates at a density of 5×10⁴. After an overnight culture, the medium was removed and replaced. The cells were infected with AAV/EGFP viruses at a multiplicity of infection (MOI) of 1×10⁵ infectious particles per cell or 5×10⁵ infectious particles per cell in antibiotics free medium. Cells were incubated for 48-72 hrs. At the corresponding time point cells were collected and analyzed for transduction efficiency by measuring eGFP expression by flow cytometry utilizing the cell analyzer BD LSRFortessa™. As shown in FIG. 1, a greater transduction efficiency was achieved at the higher MOI (5×10⁵ infectious particles per cell) for all serotypes, and transduction efficiency was similar among serotypes at this higher MOI. For the lower MOI (1×10⁵ infectious particles per cell), AAV6 exhibited the greatest transduction efficiency among the serotypes tested.

### Example 2: AAV Tropism Assessment for AAV5 and AAV6 in H1703, H520, A549, MRC5 and HepG2 Cells

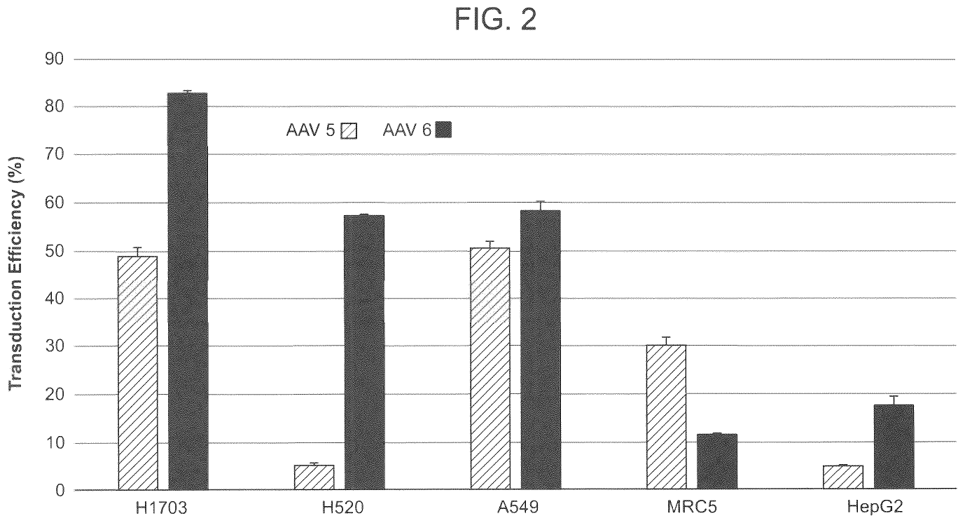

To further select the optimal serotype to transduce lung squamous cell carcinoma, the tropism of AAV5 and AAV6 was tested against cell lines from different tissue lineages (H1703 and H520 human lung squamous carcinoma; A549 human lung adenocarcinoma; MRC5 human normal lung cells; HepG2 human Hepatocellular Carcinoma). All cell lines were purchased from ATCC and cultured following their guidelines. AAV 5 and 6 that express eGFP were purchased from the Horae Gene Therapy Center at University of Massachusetts Medical School. Cells were removed from culture flasks and seeded into 12-well cell culture plates at a density of 5×10⁴. After an overnight culture, the medium was removed and replaced. The cells were infected with AAV/EGFP viruses at a multiplicity of infection (MOI) of 1×10⁵ infectious particles per cell in antibiotics free medium. Cells were incubated for 48-72 hrs. At the corresponding time point cells were collected and analyzed for transduction efficiency by measuring eGFP expression by flowcytometry utilizing the cell analyzer BD LSRFortessa™. As shown in FIG. 2, AAV6 exhibited a greater transduction efficiency for the lung cancer cell lines H1703, H520, A549 and HepG2, while AAV5 exhibited a greater transduction efficiency for the normal lung cell line MRC5.

### Example 3: AAV Tropism Assessment for AAV5 and AAV6 in a Human Xenograft Mouse Model of H1703 Non-Small Cell Lung Cancer (NSCLC)

Purpose: To determine the in vivo transduction rate of two AAV serotypes, AAV5 and AAV6, expressing firefly luciferase using the H1703 NSCLC human xenograft model in female NCG mice, and assess with bioimaging.

TABLE 4
Treatment Groups

| Gr. | N | Agent | Active dose | Route | Schedule |
|---|---|---|---|---|---|
| 1 | 8 | AAV5-fLUC | 1 × 10¹¹ | intra-tumoral | once per |
| | | | viral genomes | | day |
| | | | (vg)/animal | | |
| 2 | 8 | AAV6-fLUC | 1 × 10¹¹ | intra-tumoral | once per |
| | | | viral genomes | | day |
| | | | (vg)/animal | | |

### Procedures

CR female NCG mice were injected with 5×10⁶ H1703 tumor cells in 50% Matrigel subcutaneously in flank. The cell injection volume was 0.1 mL/mouse, and the age of the mice at the start date was 8 to 12 weeks. A pair match was performed when tumors reached an average size of 60-100 mm³ and treatment was begun. An average tumor size of ˜80 mm³ was targeted. Day 1 was defined as the day of dosing of the AAVs. Body weight of the mice was measured daily and tumor size was measured by calipers biweekly until the end of the experiment. Any individual animal with a single observation of >than 30% body weight loss or three consecutive measurements of >25% body weight loss was euthanized. The endpoint of the experiment was a mean tumor weight in the Control Group of 2000 mm³. When the endpoint was reached, all the animals were euthanized.

AAV5-fLUC and AAV6-fLUC containing the firefly luciferase gene under transcriptional control of the chicken actin promoter (CAG) were administered in PBS intratumorally in a volume of 0.05 mL/mouse on Day 1.

Whole body in vivo bioluminescent imaging was performed for a total of 7 imaging timepoints as described below:
All Groups: All Animals—Day 2 (1 day post AAV injection)
All Groups: All Animals—Day 3 (2 days post AAV injection)
All Groups: Animals 2,3,5,6,7—Day 4 (3 days post AAV injection)
All Groups: Animals 2,3,5,6,7—Day 6 (4 days post AAV injection)
All Groups: Animals 2,3,5,6,7—Day 8 (7 days post AAV injection)
All Groups: Animals 2,3,5,6,7—Day 9 (8 days post AAV injection)
All Groups: Animals 2,3,5,6,7—Day 10 (9 days post AAV injection)
All Groups: Animals 2,3,5,6,7—Day 13 (12 days post AAV injection)
All Groups: Animals 2,3,5,6,7—Day 15 (14 days post AAV injection)
All Groups: Animals 2,3,5,6,7—Day 17 (16 days post AAV injection)
All Groups: Animals 2,3,5,6,7—Day 20 (19 days post AAV injection)

The luciferase substrate (D-Luciferin) was administered at 150 mg/kg i.p. at 10 mL/kg (split into 2 injections) based upon recent body weights. Dorsolateral images were taken 10 minutes post substrate injection. Imaging was performed under anesthesia.

For ex vivo bioluminescent imaging, prior to sampling, luciferase substrate (D-Luciferin) was administered at 150 mg/kg i.p. at 10 mL/kg (split into 2 injections) based upon recent body weights. Ex vivo bioluminescent imaging of sampled tissues was performed for a total of 2 imaging timepoints as described below:
All Groups: Animals 1,4,8—Day 4 (3 days post AAV injection)
All Groups: Animals 2,5,7—Day 21 (20 days post AAV injection)

In order to capture all organs per animal, two images per animal were assumed for a total of 24 images (n=3 animals from each of 2 groups at 2 timepoints, two images per animal).

### Results

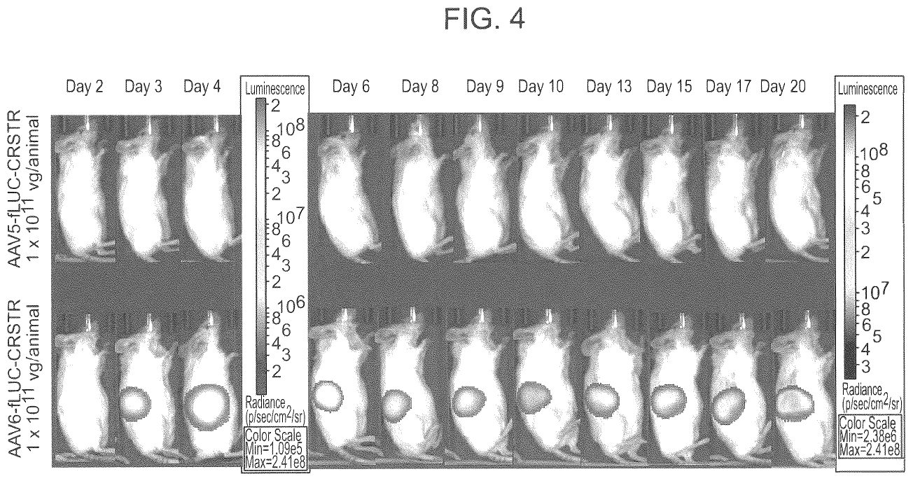

As shown in FIG. 4, mice administered the AAV6-fLUC virus exhibited greater bioluminescence starting at Day 3 relative to mice administered AAV5-fLUC. These results indicate that AAV6 exhibited a greater transduction efficiency for the lung cancer cell line H1703 in vivo relative to AAV5.

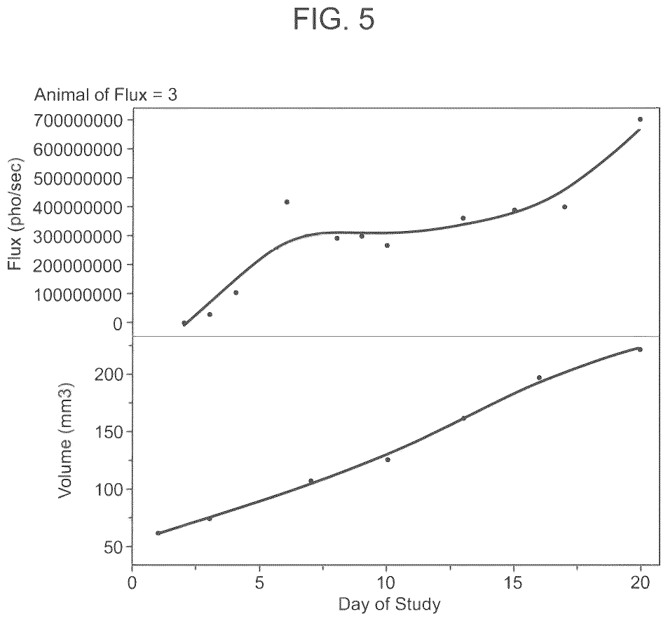

As shown in FIG. 5, both tumor volume and bioluminescence increased over time for a representative mouse implanted with H1703 squamous non-small cell lung carcinoma cells and treated intatumorally with AAV6-fLUC. These results show that bioluminescent signal and tumor volume increased steadily and did not reach a plateau over the duration of the experiment, i.e. 21 days post injection, suggesting that expression of the luciferase gene would continue beyond 21 days post injection.

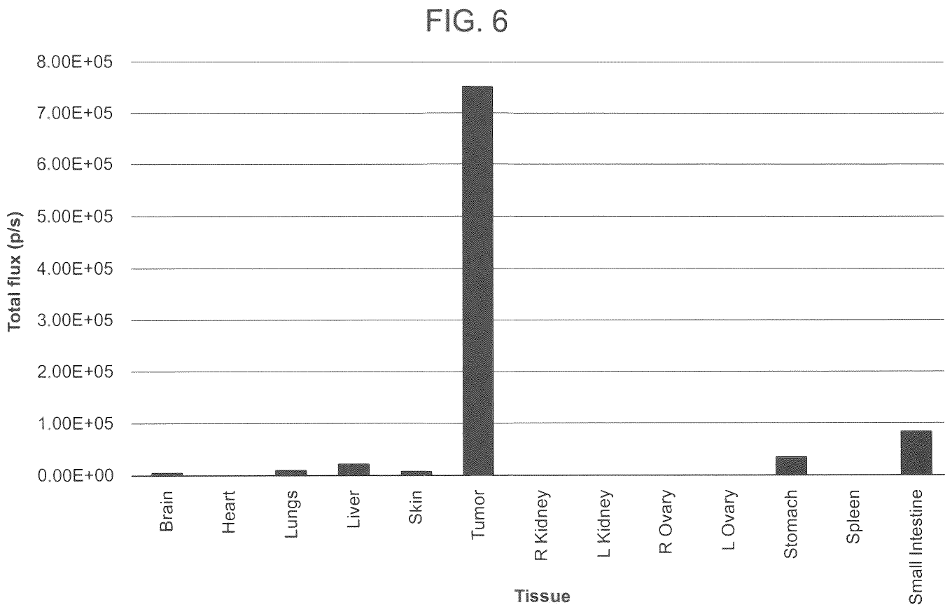

As shown in FIG. 6, bioluminescence was much greater in tumor tissue relative to other tissues on Day 21 in mice treated intatumorally with AAV6-fLUC. These results indicate that the majority of reporter gene expression remains within the tumor in which the AAV is delivered. Qualitatively, these results suggest that there would be less of a possibility of distribution of the AAV to other tissues.

### Example 4: Comparison of Transduction Efficacy in Lung Cancer Cells of AAV6 and Adenovirus

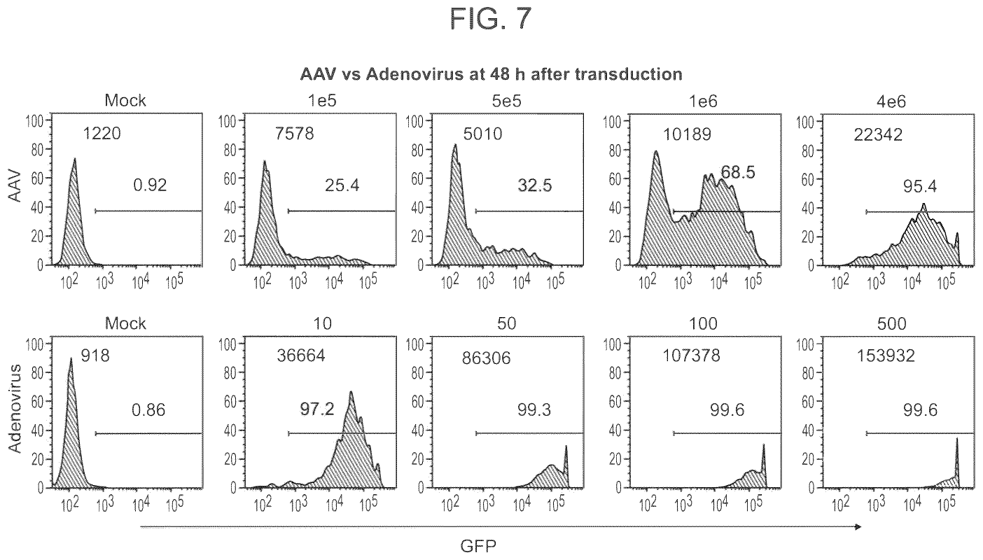

To compare the transduction efficacy in lung cancer cells between AAV6 and adenoviruses, we transduced lung cancer cells with both vectors respectively expressing GFP and evaluated the extent of GFP expression via flow cytometry at 48 h post-transduction. As shown in FIG. 7, as low as 10 multiplicity of infection (MOI) of adenovirus efficiently transduced the cells (lower panel) which outperformed the highest MOI of 4 million in AAV6 tested (upper panel). The numbers right above the scale bars represented the percentage of GFP positive cells, and the numbers at the left corners of each image indicated the mean fluorescent intensity (MFI) of GFP. The data demonstrate that the adenoviral vector has greater transduction efficiency for lung cancer cells relative to the AAV6 vector.

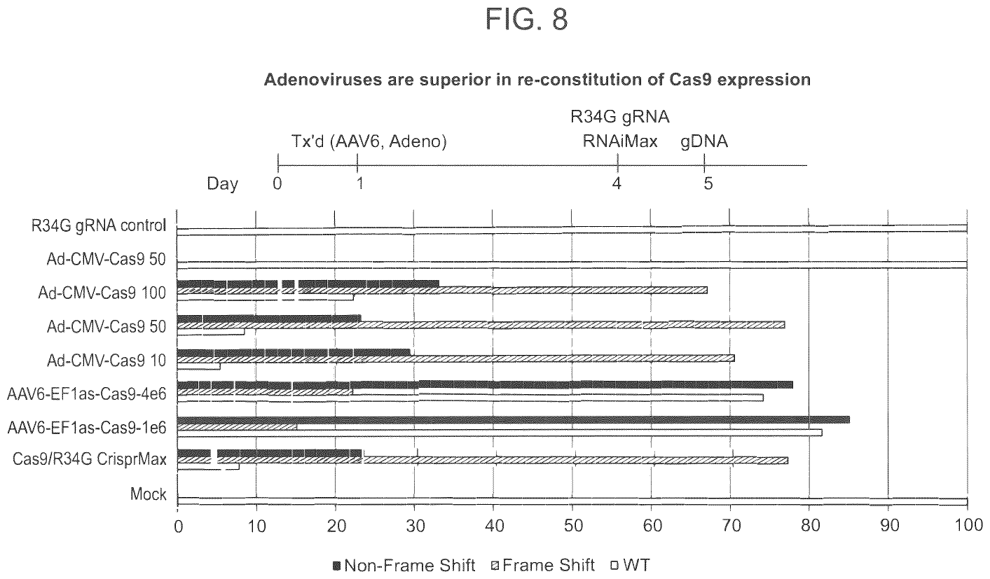

Since the size of GFP is only a fraction of the size of the Cas9 protein, we also compared the potential to use AAV6 and adenovirus to express Cas9 directly in lung cancer cells. The experiment was split into steps. In the first step, the cells were treated with nucleic acids encoding Cas9 for four days. The nucleic acids endocing Cas9 were delivered by AAV6 or adenoviral vector. In the second step, sufficient LNPs containing R34G sgRNA was delivered 4 days after infection with the AAV6 or adenovirus. The cells were transfected with R34G gRNA with the reagent RNAiMax which was purchased from Thermo Fisher Scientific. At Day 5 the gDNA from these cells was collected, and the PCR amplicons flanking the R34G mutation were Sanger sequenced. The treatment groups in the order shown in FIG. 8 (top to bottom) are described below.
R34G gRNA control: Cells treated with R34G gRNA, but not with a Cas9 construct (negative control).
Ad-CMV-Cas9 50: Cells treated with adenovirus encoding Cas9 under transcription control of the CMV promoter at an MOI of 50, but not treated with a gRNA construct (negative control).
Ad-CMV-Cas9 100: Cells treated with 1) adenovirus encoding Cas9 under transcription control of the CMV promoter at an MOI of 100, and 2) LNP mediated R34G sgRNA at day 4.
Ad-CMV-Cas9 50: Cells treated with 1) adenovirus encoding Cas9 under transcription control of the CMV promoter at an MOI of 50, and 2) LNP mediated R34G sgRNA at day 4.
Ad-CMV-Cas9 10: Cells treated with 1) adenovirus encoding Cas9 under transcription control of the CMV promoter at an MOI of 10, and 2) LNP mediated R34G sgRNA at day 4.
AAV6-EF1as-Cas9-4e6: Cells treated with 1) AAV6 encoding Cas9 under transcription control of the core EF1alpha short (as) promoter at an MOI of 4×10⁶, and 2) LNP mediated R34G sgRNA at day 4.
AAV6-EF1as-Cas9-1e6: Cells treated with 1) AAV6 encoding Cas9 under transcription control of the core EF1alpha short (as) promoter at an MOI of 1×10⁶, and 2) LNP mediated R34G sgRNA at day 4.
Cas9/R34G CrisprMax™: Cells treated with 1) LNPs containing Cas9 protein (CrisprMax™), and 2) LNP mediated R34G sgRNA at day 4 (positive control).
Mock: Cells not treated with a Cas9 construct or an sgRNA construct (negative control).

As shown in FIG. 8, adenoviral vector was superior in the re-constitution of Cas9 expression relative to the AAV6 vector where Cas9 expression was driven by the core EF1alpha short promoter, and further evidenced by the overall gene editing efficacy. The reason the EF1apha short promoter was chosen for the AAV vector to express Cas9 is that the AAV vector does not have the size capacity to contain the full CMV promoter. The data indicated that with the use of the AAV6 with multiplicity of infection (MOI) as high as 4 million, 70-80% of cells remained un-edited, while with the adenoviral vector with MOI as low as 10, only 10% of cells remained un-edited, i.e., more than 90% of the cells were gene edited with the adenoviral vector. In this experiment, the R34G sgRNA was designed based upon the R34G mutation in the human NEF2L2 gene, which encodes the NRF2 protein. These results demonstrate that adenoviral vectors have much greater efficacy than AAV vectors for expressing Cas9 in lung cancer cells.

### Example 5: Evaluation of Adenovirus Serotype 5 Vectors Encoding One or Three sgRNAs Targeting R34G NRF2 in Lung Cancer Cells, and Comparison of a Single Adenovirus 5 Vector Encoding Cas9 and R34G NRF2 sgRNAs Versus a Dual Vector System Containing Separate Adenoviral Vectors Encoding Cas9 or Three R34G NRF2 sgRNAs in Lung Cancer Cells

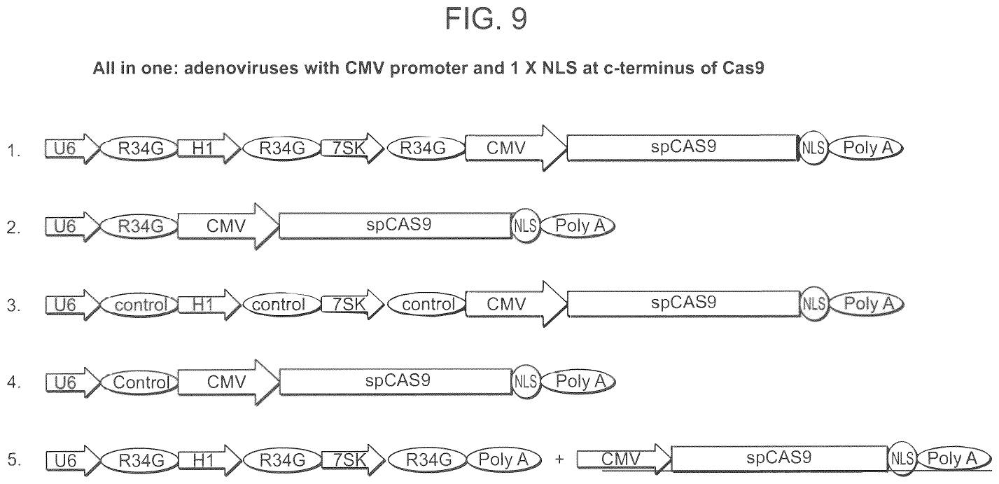

A schematic of the adenovirus serotype 5 vectors evaluated in this study is provided in FIG. 9. Treatment group 1 was a single vector encoding one copy of an R34G NRF2 sgRNA (SEQ ID NO: 128) driven by the U6 promoter, and treatment group 2 was a single vector encoding three copies of the R34G NRF2 sgRNA driven by the U6, H1 or 7SK promoter. Both of these vectors also encoded wildytpe Streptococcus pyogenes Cas9 (spCas9) driven by the CMV promoter, with one copy of an NLS fused within the C-terminus of spCas9. The spCas9 cassette was obtained from SignaGen Laboratories. Treatment groups 3 and 4 were essentially the same as 1 and 2, except that the 20 base pairs encoding the DNA binding domain of the R34G NRF2 gRNA was replaced with a scrambled DNA sequence as a negative control. Treatment group 5 was a dual vector system with one vector encoding 3 copies of the R34G NRF2 gRNAs, but lacking a spCas9 cassette; and the other vector containing an spCas9 cassette comprising a CMV promoter, but lacking a gRNA cassette.

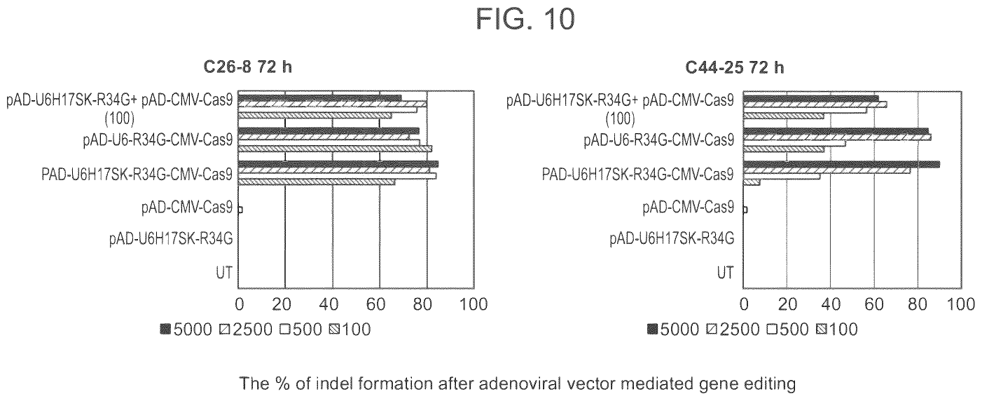

FIG. 10 shows the % of indels after lung cancer cells infected with different adenoviral vectors at different MOI PFU/ml, e.g., MOI at 100, 500, 2500, and 5000. 72 h after infection, gDNA were collected and subjected to Sanger sequencing. The % of indels was analyzed using DECODR. In FIG. 10, the % of indels was presented with lung cancer C26-8 cells on the left, and lung cancer C44-25 cells on the right. Both of these squamous non-small cell lung carcinoma cell lines were derived from the parental H1703 cell line by engineering them to contain a homozygous R34G mutation in the NRF2 gene. From the bottom to the top of each figure, the first group is labeled with UT, indicating the cells were not treated (negative control). For the 2^{nd} group from the bottom, the cells were transduced with pAD-U6H17SK-R34G only (negative control), so, no indel formation was observed. For the 3^{rd} group labeled as pAD-CMV-Cas9, only Cas9 was introduced (negative control), so there was no indel formation, or at background levels. The 4^{th} group from the bottom was pAD-U6H17SK-R34G-CMV-Cas9 (three copies of the sgRNA, plus Cas9), in which we observed increased levels of indel formation relative to the negative controls. The 5^{th} group from the bottom was pAD-U6-R34G-CMV-Cas9 (one copy of the sgRNA, plus Cas 9) infected cells. We observed similar levels of indel formation for this 5^{th} group containing one copy of the sgRNA as the 4^{th} group containing three copies of the sgRNA. The cells in the 6^{th} group were infected with dual vectors, where one vector provided 3 copies of the R34G NRF2 sgRNA, while the spCas9 was introduced by a separate pAD-CMV-Cas9 vector. This 6^{th} group resulted in similar levels of indel formation as the 4^{th} and 5^{th} groups.

In summary, vectors with one copy or three copies of R34G gRNAs showed similar gene editing efficacy in the two lung cancer cell lines transduced. Furthermore, the all in one vector performed similarly as the dual vector or better in terms of gene editing.

### Example 6: Comparison of Cis and Trans Orientation of Sequences Encoding sgRNAs Relative to Sequences Encoding Cas9

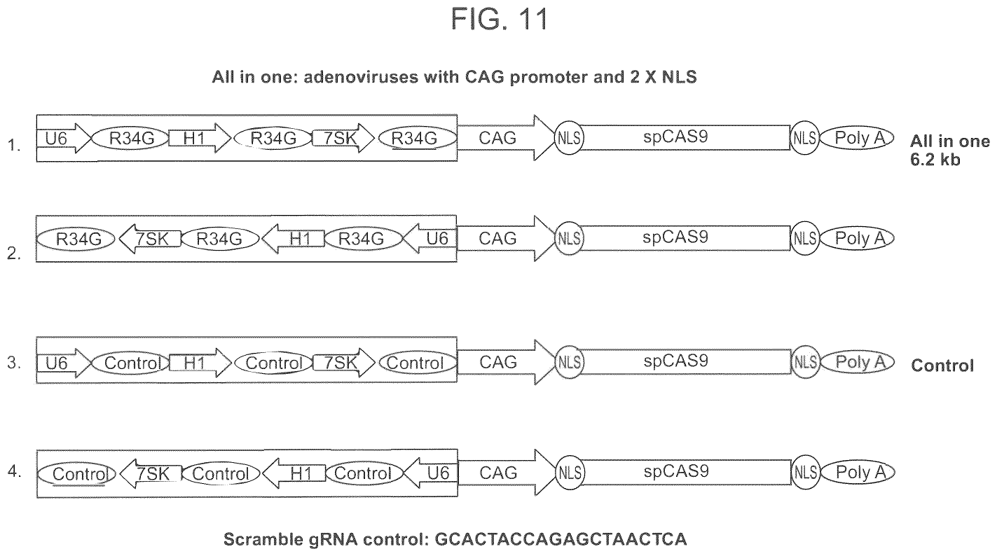

As shown in FIG. 11, 3 copies of the R34G NRF2 sgRNAs are either placed in cis (vector 1) or in trans (vector 2) orientation relative to the spCas9 cassette. In both vectors, spCas9 expression is driven by the CAG promoter, and the spCas9 cassette contains 2 copies of NLS, one fused to the N-terminus of spCas9, and one fused to the C-terminus of spCas9. For all of the constructs in FIG. 11, the spCas9 is a mutated spCas9 (enhanced specificity spCas9) in which 3 positively charged amino acids are replaced with alanine (A) to reduce off-target effects. See Slaymaker et al., Science. 2016 Jan. 1; 351 (6268): 84-88, which is incorporated by reference herein in its entirety. Vectors 3 and 4 are designed as negative control vectors in which the 20 bp encoding the DNA binding domain of the R34G NRF2 sgRNA is replaced with a scrambled DNA sequence. This negative control sgRNA sequence is indicated at the bottom of FIG. 11. The ability of these constructs to edit the NRF2 gene is evaluated in vitro to determine the effect of sgRNA coding sequence orientation relative to Cas9 orientation on gene editing activity.

### Example 7: Evaluation in Lung Cancer Cells of CMV and CAG Promoters Driving spCas9 Expression and Copy Number of Nuclear Localization Signal (NLS) in Adenovirus Serotype 5 Vectors

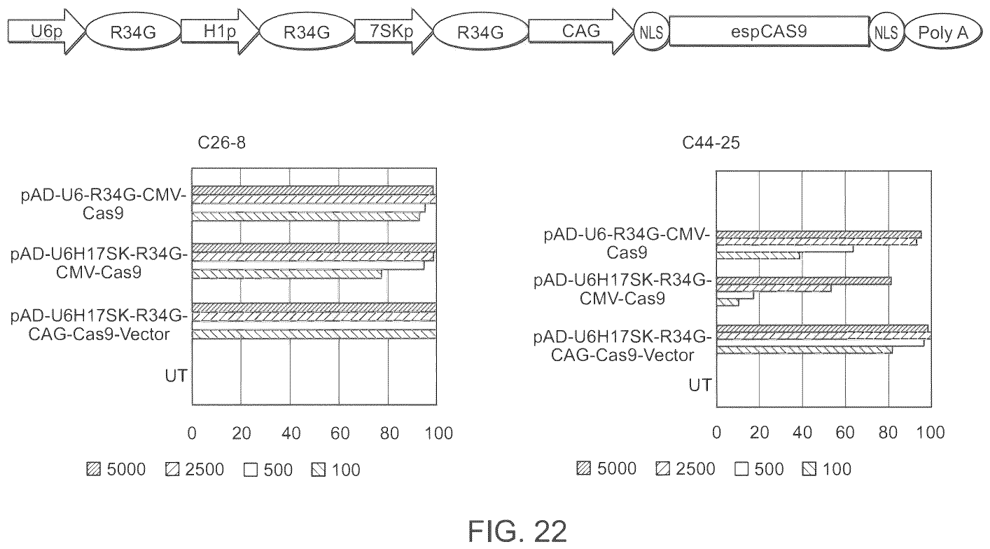

The CMV and CAG promoters were compared for driving expression of spCas9. The comparison included the constructs pAD-U6-R34G-CMV-Cas9, pAD-U6H17SK-R34G-CMV-Cas9, and pAD-U6H17SK-R34G-CAG-Cas9. The CMV promoter constructs contained one copy of NLS in the spCas9 C-terminus, while the CAG promoter construct contained two copies of NLS in spCas9, one in the N-terminus and one in the C-terminus. In addition, the CMV promoter constructs contained a wildtype spCas9, while the CAG promoter constructs contain a mutated spCas9 in which 3 positively charged amino acids were replaced with alanine (A) to reduce off-target effects. See Slaymaker et al., Science. 2016 Jan. 1; 351 (6268): 84-88. The DNA binding domain of the R34G-targeting sgRNA (R34G) is encoded by SEQ ID NO: 61. The adenoviral vectors with CMV versus CAG promoter were packaged and amplified. gDNA was collected 72 h after transduction, and subjected to Sanger sequencing. The indel formation in two engineered lung cancer cell lines (e.g., the C26-8 and C44-25 cell lines) was analyzed by DECODR software to determine gene editing activity for each construct (FIG. 22). CAG promoter-driven Cas9 construct showed 80% indel efficiency at 100 MOI while two CMV promoter-driven Cas9 constructs had 10-40% efficiencies at the equivalent MOI in the C44-25 cell lines. In the 26-8 cell line, the result was less pronounced but showed higher efficiency with the CAG promoter-driven Cas9 construct relative to the CMV promoter Cas9 construct.

### Example 8: Comparison of the CMV and CAG Promoters for Driving Cas9 Expression in H1703 Lung Cancer Cells

Two different Ad5 CRISPR/Cas9 constructs with different promoters, CMV or CAG, driving Cas9 expression were tested to target NRF2 R34G in 5 different cell models by measuring the gene editing efficacy of each vector.

### Methods

Cell Culture Conditions H1703 cells were engineered to contain the NRF2 R34G from which clonal cell lines were created: 44-25, 44-58, 64-64, 64-17. H1703 (NCI-H1703) is a lung squamous cell carcinoma cell line with a missense mutation at codon 285 (GAG→AAG) of its p53 gene. You et al., Cancer Res. 60:1009-13 (2000). NCI-H1703 has been shown to primarily express FGFR1c and to induce Erk1/2 phosphorylation upon stimulation with FGF2. Marek et al., Mol. Pharmacol. 75:196-207 (2009).

Cas9 RNP
Exon 2 gRNA 3-
(SEQ ID NO: 64)
5′ TGGAGGCAAGATATAGATCTTGG 3′
(Neh2 for NRF2 KO)
R34G ssDNA template-5′
(SEQ ID NO: 65)
TTAAAAAACATGAGCTCTCTCCTTCCTTTTTTTGTCTTAAACATAG
GACATGGATTTGATTGACATACTTTGGAGGCAAGATATAGATCTT
GGAGTAAGTGGAGAAGTATTTGACTTCAGTCAGCGACGGAAAGAG
TATGAGCTGGAAAAACAGAAAAAACTTGAAAAGGAAAGACAAGAA
CAACTCCAAAAGGAGCAAG 3′
(underlined, intended R34G mutation)

The Exon 2 gRNA 3 was designed to cleave at the D29 codon in exon 2 of the NRF2 gene in order to recreate the R34G mutation in the H1703 cell line. The gRNA was complexed with Cas9 protein to form a RNP and was used with the R34G template DNA to facilitate DNA repair and introduce an intended R34G mutation in the NRF2 gene of H1703.

All cell lines were grown and cultured per ATCC recommendations. Cell lines were tested for Mycoplasma upon thawing and before use in experiments using the MycoScope PCR Mycoplasma detection kit (Genlantis, Cat. MY01100). Patient-derived cells (PDC) were established from the NCI Patient-Derived Models Repository 073-R.

### Ad5 CRISPR/Cas9 NRF2 R34G Targeting

pAD-U6sgRNAR34G-CMVCas9 or pAD-U6sgRNAR34G-CAGCas9 were customized and purchased from Vector Biolabs Laboratories. Cells were seeded in complete media at 1×10⁵ per well into a 24-well plate 24 hrs prior to viral exposure. The next day, the complete media was removed and 200 ul of Opti-MEM with the increasing MOI (0,1,10,20,100,500) of Ad5 virus was added to cells. Cells were incubated at 37 C in 5% CO2 for 3 hrs followed by the addition of 500 ul of complete media to cells. Cells were further incubated for 72 hrs.

### Gene Editing Analysis

72 hrs post viral transduction cells were harvested and the cellular genomic DNA was isolated from each sample using the DNeasy Blood and Tissue Kit (Qiagen. Cat. 69506). The region surrounding the CRISPR target site was PCR amplified using the Q5 High-Fidelity 2×Master Mix (New England BioLabs, Cat. M0492) and the following primers:

Exon 2-
Forward primer
(SEQ ID NO: 132)
5′-CACCATCAACAGTGGCATAATGTGAA-3′
Reverse primer
(SEQ ID NO: 133)
5′-AACTCAGGTTAGGTACTGAACTCATCA-3′

The PCR reaction was purified using the QIAquick PCR Purification Kit (Qiagen, Cat. 28106) and Big Dye Terminator PCR was performed using Big Dye Terminator v3.1 (Thermofisher). PCR products were purified once more using the Big Dye Xterminator kit (Thermofisher) and then sequenced using the SeqStudio Genetic Analyzer (Applied Biosystems). Indel efficiency was assessed by using the software program, DECODR, available at decodr.org/analyze.

### Results

Increasing MOIs of 1, 10, 20, 100 and 500 were tested to assess the optimal range of gene editing efficacy to disrupt R34G mutation in NRF2 by two different adenoviral vectors that express Cas9 from different promoters, CAG or CMV. 72 h after transduction, gDNA was collected for sanger sequencing. The % of indels was determined by DECODR. FIGS. 20A-E show the % of indels from different lung cancer cells that contained the NRF2 R34G after transduction. 3 Heterozygous cell lines (PDC, 64-64, 64-17), panel A-C, and 2 homozygous (44-25, 44-58), panel D-E, NRF2 R34G genomic backgrounds were targeted. For both pAD-U6-R34G-CMV-Cas9 and pAD-U6-R34G-CAG-Cas9, there was an increase in the INDEL % as the MOI increased. In the heterozygous cell lines transduced with pAD-U6-R34G-CAG-Cas9 (panels A and B) all three cell lines have similar INDEL % as the MOI increases, with optimal % achieved at MOI of 100. The PDC showed the same % indel as the engineered cell lines, suggesting that the Ad5 would transduce successfully PDMR models. The homozygous cell lines (44-25, 44-58) in a similar manner as the heterozygous, show similar if not the same editing efficiency with increasing MOI achieving total disruption of NRF2 R34G with an MOI 100. In summary, these results show that squamous lung cancer cells that contain NRF2 R34G can be efficiently targeted by Ad5 vectors expressing CRISPR/Cas9.

### Example 9: Evaluation of the Disruption of Nrf2 Protein Expression by R34G NRF2 sgRNA in Lung Cancer Cells

Lung cancer cells H1703, C26-8 and C44-25 were plated on 6-well plates with 5×10⁵ cells per well the day before transfection. On the next day, cells were transfected with ribonucleoprotein particles (RNPs) comprising Cas9 protein and R34G gRNA in a molar ratio of 5 to 1. For each well, 50 pmol of Cas9 and 250 pmol of R34G gRNA were formulated, and the RNP was delivered by CrisprMax. 48 h after the transfection, cells for western blotting were treated with MG-132 with a concentration of 10 micromolar for 8 h before the total cell lysates were collected using a RIPA buffer with freshly added protease and phosphatase inhibitor cocktail. An equal amount of total cell lysates was loaded on 4-12% gradient mini gel, after transfer to nitrocellulous membrane, the anti-human Nrf2 rabbit polyclonal antibody was used to probe the membrane. As shown in FIG. 12, the levels of Nrf2 protein from the lanes transfected with RNP with R34G NRF2 sgRNA were significantly reduced, compared to that of RNP with a negative control sgRNA containing a scrambled DNA binding domain. The levels of Nrf2 protein were normalized with internal control GAPDH and quantitated using imageJ software. The levels of Nrf2 protein were presented as densitometry normalized to levels of Nrf2 from wild type H1703 as 100%.

### Example 10. Adenovirus 5 Transduction Efficiency Testing in Cancer Cell Lines and a Normal Human Primary Cell Line

### Goal

pAD-CMV-GFP was used to test the transduction efficiency of Ad5 to different cancer lines and Normal Human primary cell lines. By measuring the amount of eGFP expression the efficiency of transduction could be determined.

### Methods

### Cell Culture Conditions

Human cancer cell lines: NCI-H1703, A549, KYSE-410, and FADU were purchased from ATCC. Human Lung Primary cells (NHBE), were purchased from Lonza, and Human Esophageal primary cells from Cell biologics. H1703 cells were engineered to contain the NRF2 R34G mutation from which clonal cell lines were created: 44-25, 44-58, 64-64, 64-17 (see methods of Example 8 above). All cell lines were grown and cultured per the seller's recommendations. Cell lines were tested for Mycoplasma upon thawing and before use in experiments using the MycoScope PCR Mycoplasma detection kit (Genlantis, Cat. MY01100).

### Ad5 GFP Transduction

pAD-CMV-GFP (Cat. #SL100708) was purchased from SignaGen Laboratories. Cells were seeded in complete media at 1×10⁵ per well into a 24-well plate 24 hrs prior to viral exposure. The next day, the complete media was removed and 200 ul of Opti-MEM with the increasing MOI (0,1,10,20,100,500) of Ad5 virus was added to cells. Cells were incubated at 37 C in 5% CO2 for 3 hrs followed by the addition of 500 ul of complete media to cells. Cells were further incubated for 72 hrs.

### Flow Cytometry GFP Measurement

72 hrs post viral exposure cells were harvested and resuspended at 1×10⁶ cells/ml in PBS for eGFP expression by flow cytometry utilizing BD Fortezza (BD Biosciences. San Jose, CA).

### Results

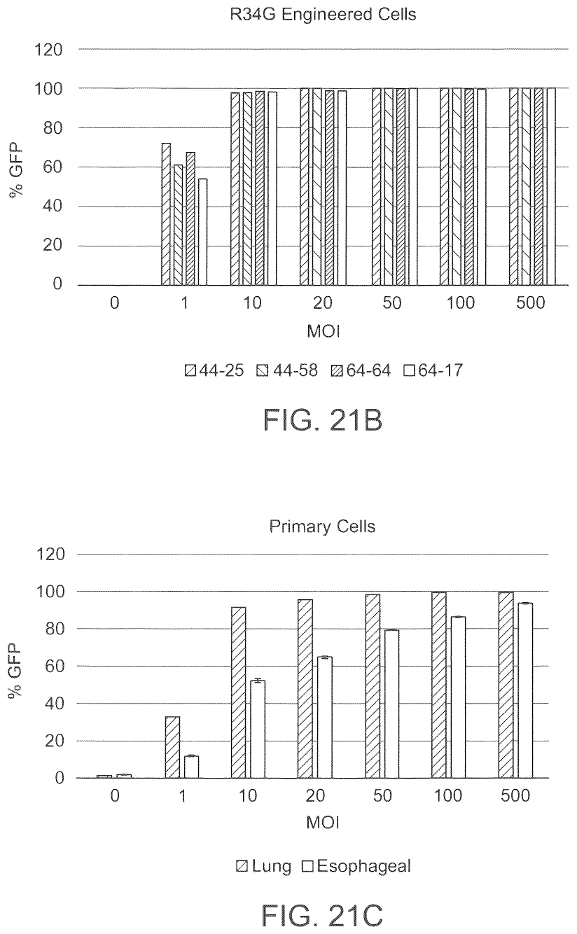

pAD-CMV-GFP was used to test the efficiency of Ad5 to test different types of cells by measuring the % of eGFP expressed, see FIG. 21A-21C. Three categories of cells were tested: stable established cancer cell lines (A). Lung squamous cancer NRF2 R34G engineered cell lines (B), and primary cells (C). H1703 squamous lung cancer cells and its derived engineered R34G cells (44-25, 44-58, 64-64, 64-17) showed a high transduction rate with 100% expression of eGFP captured at a low MOI of 10. Healthy lung primary cells followed the same trend as cancer cell lines. KYSE410 squamous esophageal cancer cells and the A549 lung adenocarcinoma cells showed the same pattern of eGFP % expression with optimal expression at an MOI Of 50. Pharynx squamous cancer cells and the healthy esophageal cell showed a gradual increase in GFP expression as the MOI increased with optimal expression achieved at MOI of 500.

### Example 11. Biodistribution of Adenovirus after Subcutaneous or Intratumoral Injection in a Human Xenograft Mouse Model of H1703 Non-Small Cell Lung Cancer (NSCLC)

Biodistribution of adenovirus after intratumoral injection is determined in mice implanted with wildtype H1703 squamous non-small cell lung carcinoma cells. The H1703 cells are implanted into the mice either subcutaneously, or orthotopically (i.e., in lung tissue), as described below.

### Subcutaneous Injection of H1703 Cells

Subcutaneous injections of H1703 cells are performed on 5 female athymic nude mice and 5 female NCG mice at age 6 weeks. This SubC injection implants tumor cells into the left flank of the fatty part of the belly. These cells are injected to grow into solid mass tumors in the murine model. The H1703 human tumor-derived cell line is injected at a concentration of 4×10⁶ in a total volume of 200 uL of sterile PBS. This injection is performed while restraining the mouse using the researcher's non-dominant hand. After injection, the mouse is returned to its home cage with ad libitum access to food and water as usual. As the cells proliferate, the tumors are measured using calipers at 3 day intervals until tumor volume has reached at least 1.5 cm in any dimension. If tumors grow beyond 1.5 cm in any dimension, the mouse is humanely euthanized using CO₂ inhalation. The purpose of this experiment is to determine suitability of tumor growth between each strain of mouse (athymic nude or NCG). Athymic nude or NCG mice are selected for the rest of the experiments depending on the success of tumor growth for each strain.

Mouse body weights are collected every 3 days to monitor animal health. If the mouse has more than 20% body weight loss at any time during this or the following experiments, the mouse is euthanized appropriately and according to the guidelines of this protocol.

TABLE 5
Animals for evaluation by subcutaneous injection

| | | treatment |
|---|---|---|
| | saline | with H1703 |
| Group | control | cancer cells |
| Athymic Nude | 5 | 5 |
| NCG | 5 | 5 |
| total requested | | 20 |

### Orthotopic Injection of H1703 Cells

H1703 cells are administered orthotopically to 5 female mice of either of the previously determined strains at age 6 weeks using a surgery to access the lung on the dorsal side. The H1703 tumor-derived cells are administered at a concentration of 1×10⁶ in a total volume of 40 μL of sterile PBS. This will be performed under isoflurane anesthesia. Animal weights are collected the day of surgery and every 3 days thereafter. After injection, the mouse is returned to its home cage with ad libitum access to food and water as usual. The tumors are measured using either IVIS bioluminescence or MRI imaging at 3 day intervals until tumor volume has reached more than 1.5 cm in any dimension. If tumors grow beyond 1.5 cm in any dimension, the mouse is humanely euthanized using CO₂ inhalation. The purpose of this experiment is to determine suitability of tumor growth in an orthotopic model using either one of the optimized strains of mouse established in the previous experiments (athymic nude or NCG).

TABLE 6
Animals requested for orthotopic injection

| | Mock | Surgical Injection |
|---|---|---|
| | Surgery | with H1703 |
| Group | Control | cancer cells |
| NOG or Athymic | 5 | 5 |
| Nude | | |
| Total Requested | | 10 |

The tumors are grown to a size of approximately 100 mm³ before injection of adenovirus. Adenovirus engineered to express luciferase under transcriptional control of a CMV promoter are administered by single injection into 4 different quadrants of the tumor. The treatment groups are described in the table below.

TABLE 7
Treatment groups

| Tumor | Virus | Total | |
|---|---|---|---|
| Type | (PFU) | volume | Mouse number |
| H1703 WT | 1 × 10⁸ | 80 μL | 20 |
| | 1 × 10⁹ | 80 μL | 20 |
| | PBS | 80 μL | 16 |

Mice are live imaged (ventrally and laterally) for luciferase activity with a IVIS luminescence camera. Five minutes before imaging, 100 ul of sodium luciferin (1.58 mg/ml; 10 μg/g body weight) is injected intraperitoneally. In vivo imaging for luciferase expression using IVIS is performed 1d, 2d, 3d, 6d, 10d, 15d, and 18d post viral injection and body weights are recorded.

Cohorts of 5 mice for Ad-CMV-Luc (or 4 mice for PBS) are euthanized, and tumor and peripheral tissues (lung, liver, blood, spleen, thymus, pancreas, stomach, intestine, kidneys, heart, ovaries, brain) are collected at 1d, 3d, 6d and 18d post viral injection timepoints. Tumor cell identity is sequence verified.

In vivo Bioluminescence Imaging (IVIS) is calculated as photon flux or RLU (ln) for each viral PFU tested.

For viral genome quantification, DNA from tumor and peripheral tissues is isolated using the Dneasy blood and tissue kit (Qiagen 69504). Quantification of viral genome in tissue and tumor specimens is performed by real-time PCR using Adeno-X qPCR Titration kit (Cat #632252).

Luciferase transgene expression in tissues is determined by lysing cells from tumor and peripheral tissues using Promega lysis buffer. Luciferase gene expression is measured using the Bright-Glo Luciferase Assay system (Promega, E2620).

Liver cytotoxicity is determined by measuring serum ALT (alanine aminotransferase) and AST (aspartate aminotransferase) using ELISA to account for virus-induce damage to hepatocytes.

### Example 12-Delivery of Adenoviral Vector (fLuc) in H1703 (44-25) Subcutaneous Xenograft Model

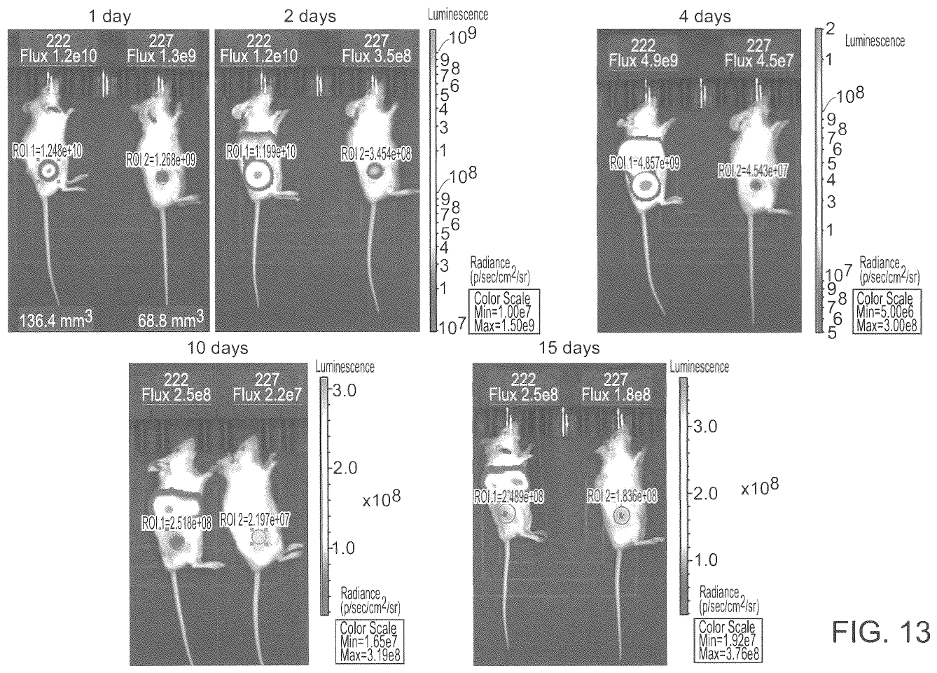

Adenovirus containing the firefly luciferase transgene was used to assess intratumoral delivery and expression within a xenograft mouse model. CRISPR-engineered human lung squamous cell carcinoma cell line, H1703 44-25, was implanted subcutaneously. Once tumors reached 60-150 mm³ in size, mice were injected intratumorally with 1.5e9 pfu of Ad5-CMV-fLuc (SignaGen Laboratories) to 3 sites within the tumor. Bioluminescence imaging was conducted on mice 1-, 2-, 4-, 10- and 16-days post injection. FIG. 13 displays time course of the bioluminescent signal post intratumoral injection. Signal from each tumor is quantified by establishing a region of interest (ROI) of the tumor. The ROI value listed as total flux photons of each tumor. One day post intratumoral injection in both mice with different sized tumors created a strong signal with comparable ROIs. The signal increased and plateaued over the course of 16 days.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of 5×10⁶ cells (human lung squamous cell-derived H1703 clone 44-25) in BD matirgel (volume ratio 1:1). Tumor growth was measured and estimated using a caliper and calculated as volume (mm³)=(length [mm]×(width [mm]) 2×0.5. When tumors reached ˜60-150 mm³, mice were separated to experimental groups. Adenoviral injections consisted of 1.5e⁹ pfu in a total volume of 30 μL of Ad5-CMV-fLuc purchased from SignaGen Laboratories.

### Bioluminescence Imaging

Bioluminescence imaging was performed with an IVIS Spectrum imaging system (Caliper Life Sciences). Mice were administered intraperitoneally with d-luciferin (Promega) at a dose of 150 mg/kg. Five minutes after receiving d-luciferin, mice were anesthetized in a chamber with 3% isoflurane and placed on the imaging platform while being maintained on 3% isoflurane via a nose cone. Mice were imaged at 15 minutes post administration of d-luciferin using an exposure time of 1 second or longer. Bioluminescence values were quantified by measuring photon flux (photons/second) in the region of interest where bioluminescence signal emanated using the Living IMAGE Software provided by Caliper (Hopkinton, MA).

### Example 13-Delivery of Adenoviral Vector Targeting Nrf2 in H1703 (44-25) Subcutaneous Xenograft Model

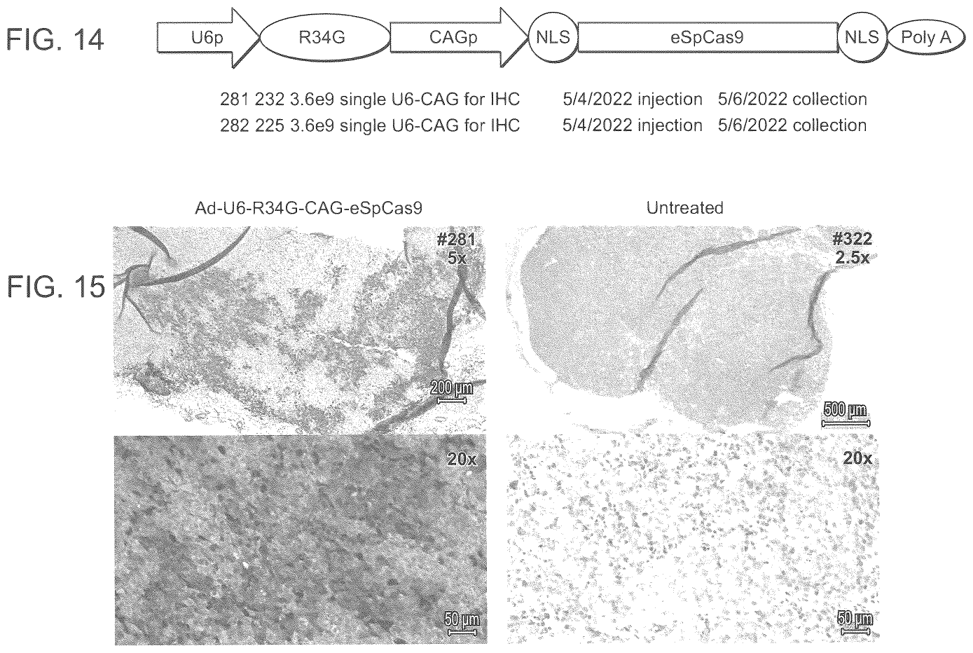

Adenovirus containing CRISPR/Cas9 targeting NRF2 was used to assess intratumoral delivery and expression within a xenograft mouse model. CRISPR-engineered human lung squamous cell carcinoma cell line, H1703 44-25, was implanted subcutaneously. Once tumors reached 60-150 mm³ in size, mice were injected intratumorally with 3.6e⁹ pfu of Ad-U6-R34G-CAG-eSpCas9 or without any treatment. FIG. 14 depicts the schematic diagram of Ad-U6-R34G-CAG-eSpCas9 used with the U6 promoter driving expression of an R34G-targeting sgRNA. The DNA binding domain of the R34G-targeting sgRNA is encoded by SEQ ID NO: 61. Forty-eight hours post intratumoral injection, tumors were collected for immunohistochemistry staining and analysis. Immunostained tumor tissues were imaged and analyzed for localization and expression of Cas9. As shown in FIG. 15, the top panel of images displays 5× and 2.5× magnification of treated and untreated tumors followed by 20× magnification in the bottom panel of images. DAB staining was utilized to visualize localization of the signal within the tumor section. As seen in Ad-U6-R34G-CAG-eSpCas9 treated tumor tissues, there is signal (dark brown reaction) throughout the tumor section as opposed to the clear, even image obtained from untreated tumor sections.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of 5×10⁶ cells (human lung squamous cell-derived H1703 clone 44-25) in BD matirgel (volume ratio 1:1). Tumor growth was measured and estimated using a caliper and calculated as volume (mm³)=(length [mm]×(width [mm])²×0.5. When tumors reached ˜60-150 mm³, mice were separated to experimental groups. Adenoviral injections consisted of 3.6c9 pfu in a total volume of 30 μL of Ad-U6-R34G-CAG-eSpCas9 purchased from Vector Biolabs.

### Immunohistochemistry

Tumor tissues were fixed with 4% paraformaldehyde in 1×PBS overnight at 4° C. Cas9 labelling was performed using immunohistochemistry on fixed tumor tissues. Tumors were frozen and sliced at 10 μm and mounted directly to slides. After blocking with 5% Normal Goat Serum (Vector), rabbit anti-Cas9 primary antibody (Abcam) was applied directly to each section in incubation buffer (1% BSA in PBS) at a 1:100 dilution ratio. The primary antibody was incubated overnight at 4° C. in humidity chambers to prevent drying. Goat anti-rabbit biotinylated secondary (Vector) was applied directly to each section in incubation buffer at a 1:200 dilution ratio. The secondary antibody incubated at room temperature for 1 hour. ABC Elite HRP kit (Vector) was used for detection with methods following the kit's protocol. DAB was used to develop the stain and slides were counterstained with Hemotoxylin. Slides were then dehydrated in ethanol/xylene and permanently mounted with Acrytol. Slides were imaged on LEICA DMIL LED microscope.

### Example 14-Delivery of Adenoviral Vector Targeting Nrf2 in an NCI PDMR PDX Human Lung Squamous Cell Carcinoma Mouse Model

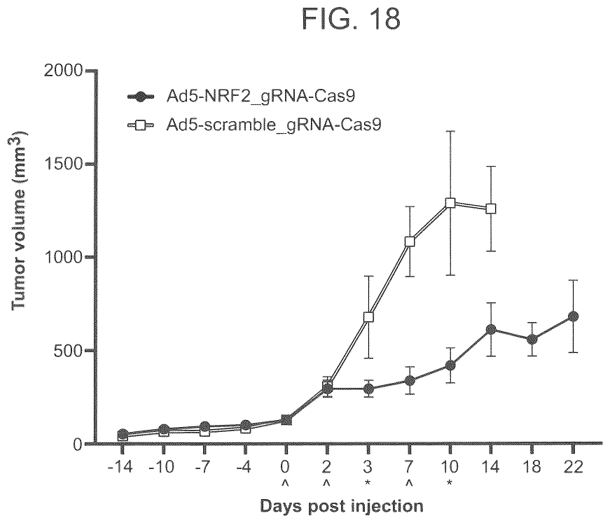

Adenovirus containing CRISPR/Cas9 targeting R34G mutant NRF2 was used to assess intratumoral delivery and expression within a patient-derived xenograft mouse model. Human lung squamous cell carcinoma tumor fragments (NCI PMDR) were implanted subcutaneously. Once tumors reached 60-150 mm³ in size, mice were injected intratumorally with 3.6e⁹ pfu of Ad-U6-R34G-CAG-eSpCas9 (schematic diagram shown in FIG. 14) or without any treatment. Forty-eight hours post intratumoral injection, tumors were collected for immunohistochemistry staining and analysis. Immunostained tumor tissues were imaged and analyzed for localization and expression of Cas9. As shown in FIG. 16, the top panel of images displays 2.5× magnification of treated and untreated tumors followed by 20× magnification in the bottom panel of images. DAB staining was utilized to visualize localization of the signal within the tumor section. As seen in Ad-U6-R34G-CAG-eSpCas9 treated tumor tissues, there is signal (dark brown reaction) throughout the tumor section as opposed to the clear, even image obtained from untreated tumor sections.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of PDX fragments derived from human lung squamous cell carcinoma tumor (obtained from NCI PDMR, specimen ID 073-R). Tumor growth was measured and estimated using a caliper and calculated as volume (mm³)=(length [mm]×(width [mm])²×0.5. When tumors reached ˜60-150 mm³, mice were separated to experimental groups. Adenoviral injections consisted of 3.6e⁹ pfu in a total volume of 30 μL of Ad-U6-R34G-CAG-eSpCas9 purchased from Vector Biolabs.

### Immunohistochemistry

Tumor tissues were fixed with 4% paraformaldehyde in 1×PBS overnight at 4° C. Cas9 labelling was performed using immunohistochemistry on fixed tumor tissues. Tumors were frozen and sliced at 10 μm and mounted directly to slides. After blocking with 5% Normal Goat Serum (Vector), rabbit anti-Cas9 primary antibody (Abcam) was applied directly to each section in incubation buffer (1% BSA in PBS) at a 1:100 dilution ratio. The primary antibody was incubated overnight at 4° C. in humidity chambers to prevent drying. Goat anti-rabbit biotinylated secondary (Vector) was applied directly to each section in incubation buffer at a 1:200 dilution ratio. The secondary antibody incubated at room temperature for 1 hour. ABC Elite HRP kit (Vector) was used for detection with methods following the kit's protocol. DAB was used to develop the stain and slides were counterstained with Hemotoxylin. Slides were then dehydrated in ethanol/xylene and permanently mounted with Acrytol. Slides were imaged on LEICA DMIL LED microscope.

### Example 15-Delivery and Efficacy of an Adenoviral Vector Targeting Nrf2 in an H1703 (44-25) Mouse Model

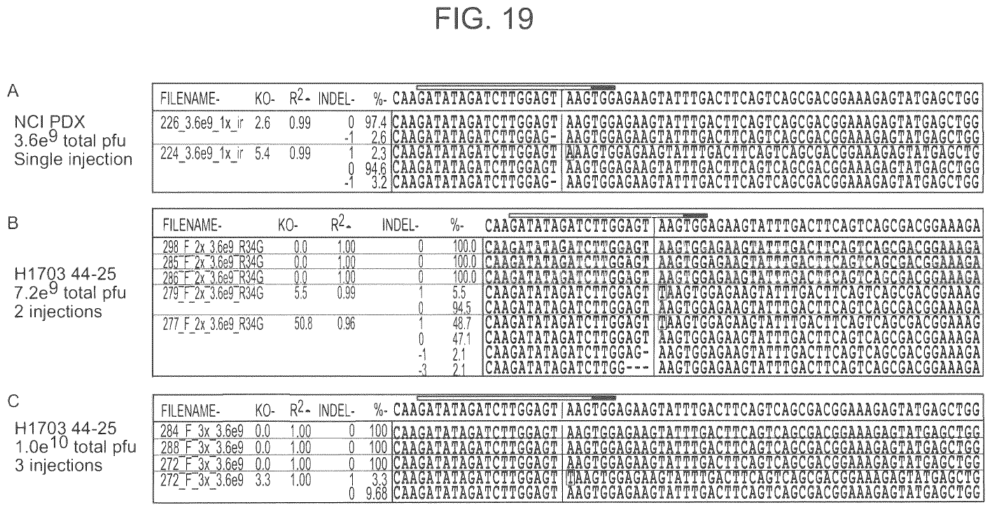

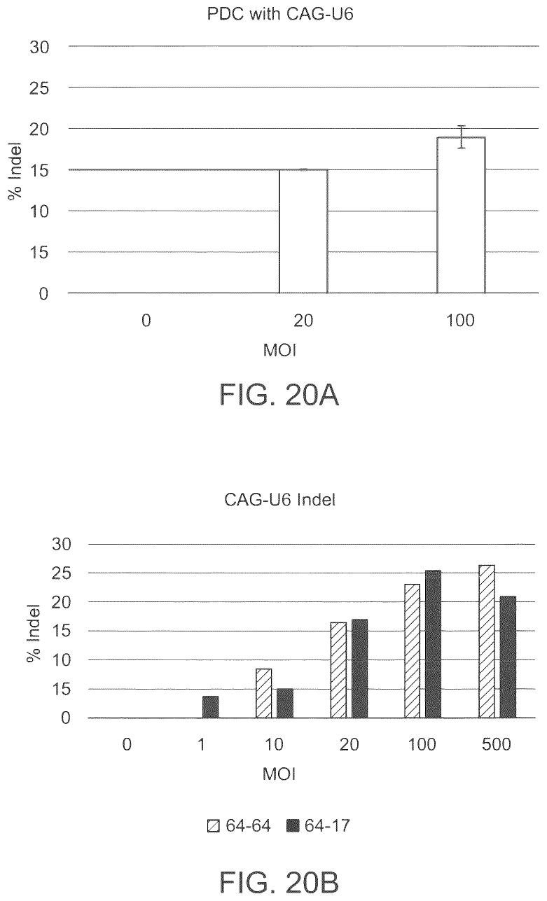

Adenovirus containing CRISPR/Cas9 targeting NRF2 was used to assess intratumoral delivery and efficacy within a xenograft mouse model. CRISPR-engineered human lung squamous cell carcinoma cell line, H1703 44-25 (which contains the R34G NRF2 mutation) was implanted subcutaneously. Once tumors reached 60-150 mm³ in size, mice were injected intratumorally with 3.65e⁹ pfu of A) Ad-U6H17SK-R34G-CAG-eSpCas9 (Vector Biolabs) or B) Ad-U6H17SK-scramble-CAG-eSpCas9 (Vector Biolabs) (as shown in the schematic diagram in FIG. 17) on days 0, 2, and 7 as denoted in FIG. 18 on the x axis by \hat{ } for a total of 1.08e¹⁰ pfu. The DNA binding domain of the R34G sgRNA is encoded by SEQ ID NO: 61, and the DNA binding domain of the “scramble” sgRNA is encoded by SEQ ID NO: 131. FIG. 17A shows the schematic of Ad-U6H17SK-R34G-CAG-eSpCas9 with U6, H1 and 7SK promoters each driving expression of an R34G-targeting sgRNA (DNA binding domain encoded by SEQ ID NO: 61), followed by the chicken beta actin (CAG) promoter driving enhanced SpCas9 expression. FIG. 17B shows the schematic of Ad-U6H17SK-scramble-CAG-eSpCas9 with U6, H1 and 7SK promoters each driving expression of a scramble sgRNA (DNA binding domain encoded by SEQ ID NO: 131) followed by the chicken beta actin (CAG) promoter driving enhanced SpCas9 expression. The scramble sgRNA was used as a negative control. Mice were treated with 12.5 mg/kg carboplatin and 5 mg/kg paclitaxel (intravenous injections) on days 3 and 10 as denoted by * on the x axis. Animals were monitored for 22 days post injection. The results showed that mice treated with NRF2-targeting adenovirus with combined therapy showed a greater reduction in tumor size at sacrifice. See FIG. 18. Mice treated with scramble sgRNA adenovirus with combined therapy reached endpoint tumor size by day 14. All tumors were collected at sacrifice for further analysis.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of 5×10⁶ cells (human lung squamous cell-derived H1703 clone 44-25) in BD matirgel (volume ratio 1:1). Tumor growth was measured and estimated using a caliper and calculated as volume (mm³)=(length [mm]×(width [mm])²×0.5. When tumors reached ˜60-150 mm³, mice were separated to experimental groups. Adenoviral injections consisted of 3 doses of 3.6e⁹ pfu in a 30 μL of Ad-U6H17SK-R34G-CAG-eSpCas9 or Ad-U6H17SK-scramble-CAG-eSpCas9 purchased from Vector Biolabs and administered every other day. Two doses of 12.5 mg/kg of carboplatin and 5 mg/kg of paclitaxel were administered intravenously in xenografted mice. In some experiments, when tumors reached 1500 mm³ in size, mice were euthanized and tumors were surgically excised and processed for histopathology or genomic DNA.

### Example 16—Targeting Efficiency of Intratumoral Delivery of Cas9 Using an Adenoviral Vector

To assess activity and efficiency of NRF2-targeting adenovirus, tumors from the experiment described in Example 15 above were excised from experimental mice and genomic DNA was isolated. Once the genomic DNA was isolated, the region of interest of NRF2 was PCR amplified and the PCR amplicon was used for Sanger sequencing. Sanger sequencing from each sample was assessed for indel efficiency at the CRISPR target site, using DECODR software to deconvolute sequence chromatograms. FIG. 19 shows the DECODR analysis output of each sample per experiments. FIG. 19A displays sequencing results from example 15, which consisted of a single injection of 3.6e⁹ pfu of Ad-U6-R34G-CAG-eSpCas9. The results show two samples with 2.6% and 5.4% CRISPR efficiency. FIG. 19B displays data from experiments not listed but executed similarly to those of Example 15. The results show 5 samples with 0%, 5.5% and 50.8% CRISPR efficiency. FIG. 19C displays sequencing results from example 15. The results show 4 samples with 0% and 3.3% CRISPR efficiency.

### Materials and Methods

### Gene Editing Analysis

Cellular genomic DNA was isolated from each clonal cell line using the DNeasy Blood and Tissue Kit (Qiagen). The region surrounding the CRISPR target site was PCR amplified using the Q5 High-Fidelity 2× Master Mix (New England BioLabs) (FWD primer-SEQ ID 03, REV primer-SEQ ID 04). The PCR reaction was purified using the QIAquick PCR Purification Kit (Qiagen) and Big Dye Terminator PCR was performed using Big Dye Terminator v3.1 (Thermofisher). PCR products were purified once more using the Big Dye Xterminator kit (Thermofisher) and then sequenced using the SeqStudio Genetic Analyzer (Applied Biosystems). Sequence analysis was conducted using the software program, DECODR, available at decodr.org/analyze.

1. An adenovirus comprising a polynucleotide, wherein the polynucleotide comprises:
(a) a first DNA molecule encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in an NRF2 gene; and
(b) a first promoter that is operably linked to the DNA molecule.

2. The adenovirus of claim 1, wherein the gene is a wildtype gene.

3. The adenovirus of claim 1, wherein the gene is a variant gene.

4.-8. (canceled)

9. The adenovirus of claim 1, further comprising:
(a) a second DNA molecule encoding the gRNA; and
(b) a second promoter that is operably linked to the second DNA molecule.

10. The adenovirus of claim 9, further comprising:
(a) a third DNA molecule encoding the gRNA; and
(b) a third promoter that is operably linked to the third DNA molecule.

11. The adenovirus of claim 1, wherein the DNA-binding domain of the gRNA comprises the nucleic acid sequence of SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, or SEQ ID NO: 126, or a biologically active fragment thereof.

12. The adenovirus of claim 1, wherein one or more of the first, second and third DNA molecules encoding the gRNA comprises the nucleic acid sequence of SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 59, SEQ ID NO: 60, SEQ ID NO: 61, SEQ ID NO: 62, SEQ ID NO: 63, SEQ ID NO: 84 or SEQ ID NO: 113, or a biologically active fragment thereof.

13.-16. (canceled)

17. The adenovirus of claim 1, further comprising a DNA molecule that encodes a Clustered Regularly Interspaced Short Palindromic Repeat (CRISPR)-associated endonuclease protein or a fragment thereof.

18. The adenovirus of claim 17, wherein the CRISPR-associated endonuclease is a class 2 CRISPR-associated endonuclease.

19. The adenovirus of claim 18, wherein the class 2 CRISPR-associated endonuclease is Cas9 or Cas12a.

20. The adenovirus of claim 17, wherein the DNA molecule that encodes the CRISPR-associated endonuclease or fragment thereof is operably linked to a tissue-specific promoter or a constitutive promoter.

21. The adenovirus of claim 20, wherein the tissue-specific promoter is a cancer tissue specific promoter.

22. The adenovirus of claim 21, wherein the cancer tissue-specific promoter is a human telomerase reverse transcriptase (hTERT) promoter.

23. The adenovirus of claim 20, wherein the tissue-specific promoter is a lung tissue-specific promoter.

24. The adenovirus of claim 20, wherein the constitutive promoter is a CMV promoter or a chicken beta-actin promoter (CAG) promoter.

25. The adenovirus of claim 17, wherein the DNA molecule that encodes the CRISPR-associated endonuclease is operably linked to at least one nucleus localization signal (NLS).

26. The adenovirus of claim 1, wherein the adenovirus is serotype 5.

27.-29. (canceled)

30. A pharmaceutical composition comprising:
a) a first adenovirus comprising a DNA molecule encoding a guide RNA (gRNA), wherein the gRNA comprises a DNA-binding domain and a Clustered Regularly Interspaced Short Palindromic Repeat (CRISPR)-associated endonuclease protein-binding domain, and the DNA-binding domain is complementary to a target sequence in an NRF2 gene;
b) a second adenovirus comprising a DNA molecule encoding a Clustered Regularly Interspaced Short Palindromic Repeat (CRISPR)-associated endonuclease protein or a fragment thereof; and
c) a pharmaceutically acceptable carrier,
wherein the first adenovirus does not comprise a DNA molecule encoding a CRISPR-associated endonuclease protein or fragment thereof, and the second adenovirus does not comprise a DNA molecule encoding a gRNA.

31.-39. (canceled)

40. A method of treating cancer in a subject, the method comprising administering to the subject a therapeutically effective amount of the pharmaceutical composition of claim 30 to the subject.

41. (canceled)

42. The method of claim 40, wherein the cancer is selected from the group consisting of lung cancer, pancreatic cancer, melanoma, esophageal squamous cancer (ESC), head and neck squamous cell carcinoma (HNSCC), and breast cancer.

43. The method of claim 42, wherein the cancer is lung cancer is a non-small cell lung cancer (NSCLC) squamous-cell carcinoma.

44.-47. (canceled)

48. The method of claim 40, further comprising administering one or more chemotherapeutic agents to the subject.

49. The method of claim 48, wherein the one or more chemotherapeutic agents are selected from the group consisting of cisplatin, vinorelbine, carboplatin, paclitaxel, docetaxel, cabazitaxel and a combination thereof.

50.-57. (canceled)
